(19) United States
(12) Patent Application Publication
(10) Pub. No.: US 2022/0008515 A1
(43) Pub. Date: Jan. 13, 2022

(54) METHOD OF TREATING A TUMOR WITH A COMBINATION OF IL-7 PROTEIN AND AN IMMUNE CHECKPOINT INHIBITOR

(71) Applicants: NeoImmuneTech, Inc., Rockville, MD (US); Genexine, Inc., Seongnam-si, Gyeonggi-do (KR)

(72) Inventors: Seung-Woo LEE, Pohang (KR); Ji-Hae KIM, Pohang (KR); Saet-byeol JO, Pohang (KR); Han Wook PARK, Pohang (KR); Donghoon CHOI, Seongnam-si (KR); Byung Ha LEE, Rockville, MD (US); Young Chul SUNG, Gyeongsangbuk-do (KR); Se Hwan YANG, Seongnam-si (KR)

(73) Assignees: NeoImmuneTech, Inc., Rockville, MD (US); Genexine, Inc., Seongnam-si, Gyeonggi-do (KR)

(21) Appl. No.: 17/294,314

(22) PCT Filed: Nov. 15, 2019
(86) PCT No.: PCT/US2019/061813

§ 371 Date: May 14, 2021

Related U.S. Application Data
(60) Provisional application No. 62/768,355, filed on Nov. 16, 2018.
(60) Provisional application No. 62/826,734, filed on Mar. 29, 2019.
(60) Provisional application No. 62/896,484, filed on Sep. 5, 2019.

(51) Int. Cl. A61K 38/20; A61K 39/395; A61P 35/00; A61K 9/00

(52) U.S. Cl. CPC A61K 38/2046; A61K 9/0019; A61P 35/00; A61K 39/3955

(57) ABSTRACT

The present disclosure relates to methods of treating a cancer (or a tumor) with an IL-7 protein in combination with an immune checkpoint inhibitor, such as a PD-1 antagonist (e.g., anti-PD-1 antibody) or a CTLA-4 antagonist (e.g., anti-CTLA-4 antibody).

### CROSS-REFERENCE TO RELATED APPLICATIONS

This PCT application claims the priority benefit of U.S. Provisional Application Nos. 62/768,355, filed Nov. 16, 2018; 62/826,734, filed Mar. 29, 2019; and 62/896,484, filed Sep. 5, 2019, each of which is herein incorporated by reference in its entirety.

### REFERENCE TO SEQUENCE LISTING SUBMITTED ELECTRONICALLY VIA EFS-WEB

The content of the electronically submitted sequence listing in ASCII text file (Name: 4241_002PC03_SequenceListing_ST25.txt; Size: 78,087 bytes; and Date of Creation: Nov, 14, 2019) filed with the application is herein incorporated by reference in its entirety.

### BACKGROUND OF THE DISCLOSURE

Human cancers harbor numerous genetic and epigenetic alterations, generating neoantigens potentially recognizable by the immune system. Sjoblom et al., Science 314:268-74 (2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Cancer immunotherapy has become well-established in recent years and is now one of the more successful treatment options available for many cancer patients. Scott, A. M., et al., Cancer Immun 12:14 (2012). Aside from targeting antigens that are involved in cancer cell proliferation and survival, antibodies can also activate or antagonize immunological pathways that are important in cancer immune surveillance. And, intensive efforts have led to the successful development of several immune checkpoint pathway inhibitors, some of which have been approved by the Food and Drug Administration, e.g., anti-CTLA-4 antibody: ipilimumab (YERVOY®); anti-PD-1 antibody: nivolumab (OPDIVO®), pembrolizumab (KEYTRUDA®); and anti-PD-L1 antibody: atezolizumab (TECENTRIQ®), durvalumab (IMFINZI®), avelumab (BAVENCI®).

Despite such advances, patients with certain malignant tumors (e.g., metastatic or refractory solid tumors) continue to have very poor prognosis. Only a subset of such patients actually experience long-term cancer remission, with many patients either not responding or initially responding but eventually developing resistance to the antibodies. Sharma, P., et al., Cell 168(4): 707-723 (2017). Moreover, many cancer patients are lymphopenic, as many of the available standard of care cancer treatments (e.g., chemotherapy and radiation therapy) are known to cause lymphopenia. Grossman, S. A., et al., J Nall Compr Canc Netw 13(10):1225-31 (2015). Checkpoint inhibitors, such as anti-PD-1 antibodies, have been shown to have limited efficacy in such cancer patients. Yarchoan, M., et al., J Clin Oncol 35:e14512 (2017). Accordingly, there remains a need for new treatment options with acceptable safety profile and high efficacy in cancer patients, including those with lymphopenia.

### SUMMARY OF THE DISCLOSURE

Provided herein are methods of treating a tumor in a human subject in need thereof, comprising administering to the subject an effective amount of an interleukin-7 (IL-7) protein in combination with an effective amount of a Programmed Death-1 (PD-1) pathway inhibitor, wherein a tumor volume is decreased in the subject after the administration compared to a reference tumor volume after administration of either the PD-1 pathway inhibitor alone or IL-7 protein alone. In some aspects, the tumor volume is decreased by at least about 5%, at least about 10%, at least about 20%, at least about 30%, at least about 40%, at least about 50%, at least about 60%, at least about 70%, at least about 80%, at least about 90%, or at least about 100% after the administration.

In some aspects, a method of the present disclosure increases a number of tumor infiltrating lymphocytes (TILs) in the tumor after the administration compared to a number of TILs in a tumor after administration of either the PD-1 pathway inhibitor alone or IL-7 protein alone. In certain aspects, the TILs are CD4⁺ TILs. In other aspects, the TILs are CD8⁺ TILs. In some aspects, the number of TILs is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration.

In some aspects, a human subject exhibits a lymphopenia prior to the administration (i.e., as described herein).

Also provided herein are methods of treating a tumor in a subject in need thereof, comprising administering to the subject an effective amount of an interleukin-7 (IL-7) protein in combination with an effective amount of a Programmed Death-1 (PD-1) pathway inhibitor, wherein the subject exhibits a lymphopenia.

In some aspects, the human subject exhibiting lymphopenia has T lymphopenia, B lymphopenia, and/or NK lymphopenia. In some aspects, the lymphopenia is caused by or associated with the tumor. In certain aspects, the lymphopenia is caused by or associated with a previous therapy for the tumor. In further aspects, the lymphopenia is caused by an infection, chronic failure of the right ventricle of the heart, Hodgkin's disease and cancers of the lymphatic system, leukemia, a leak or rupture in the thoracic duct, side effects of prescription medications including anticancer agents (e.g., chemotherapy), antiviral agents, and glucocorticoids, malnutrition resulting from diets that are low in protein, radiation therapy, uremia, autoimmune disorders, immune deficiency syndromes, high stress levels, trauma, thymectomy, or a combination thereof. In certain aspects, the lymphopenia is idiopathic. In certain aspects, the lymphopenia comprises an idiopathic CD4 positive T-lymphocytopenia (ICL), acute radiation syndrome (ARS), or a combination thereof.

In some aspects, the lymphopenia is characterized by a circulating blood total lymphocyte count that is less than by at least about 5%, at least about 10%, at least about 20%, at least about 30%, at least about 40%, at least about 50%, at least about 60%, at least about 70%, at least about 80%, at least about 90%, or at least about 100% compared to a circulating blood total lymphocyte count in a corresponding subject who does not exhibit a lymphopenia. In certain aspects, the lymphopenia is characterized by a circulating blood total lymphocyte count of less than about 1,500 lymphocytes/μL, less than about 1,000 lymphocytes/μL, less than about 800 lymphocytes/μL, less than about 500 lymphocytes/μL, or less than about 200 lymphocytes/μL.

In some aspects, a number of tumor infiltrating lymphocytes (TILs) in the tumor of a subject exhibiting a lymphopenia is increased after the administration compared to a number of TILs in a tumor after administration of either the PD-1 pathway inhibitor alone or IL-7 protein alone). In certain aspects, the number of TILs is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration. In some aspects, the TILs are CD4⁺ T cells. In some aspects, the TILs are CD8⁺ T cells.

In some aspects, an IL-7 protein is not a wild type IL-7.

In some aspects, an IL-7 protein comprises an oligopeptide consisting of 1 to 10 amino acid residues. In certain aspects, the oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine. In some aspects, the oligopeptide is methionine-glycine-methionine.

In some aspects, an IL-7 protein comprises a half-life extending moiety. In certain aspects, a half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some aspects, a half-life extending moiety is an Fc. In certain aspects, the Fc is a hybrid Fc, comprising a hinge region, a CH2 domain, and a CH3 domain, wherein the hinge region comprises a human IgD hinge region, wherein the CD2 domain comprises a part of human IgD CH2 domain and a part of human IgG4 CH2 domain, and wherein the CH3 domain comprises a part of human IgG4 CH3 domain.

In some aspects, an IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6 and 15-25.

In some aspects, a PD-1 pathway inhibitor that can be used with the present methods comprises an anti-PD-1 antibody or an anti-PD-L1 antibody. In certain aspects, the anti-PD-1 antibody comprises nivolumab, pembrolizumab, MEDI0608, AMP-224, PDR001, BGB-A317, or any combination thereof. In some aspects, the anti-PD-L1 antibody comprises BMS-936559, MPDL3280A, MEDI4736, MSB0010718C, or any combination thereof.

In some aspects, the IL-7 protein and the PD-1 pathway inhibitor are administered concurrently. In other aspects, the IL-7 protein and the PD-1 pathway inhibitor are administered sequentially. In certain aspects, the IL-7 protein is administered to the subject prior to administering the PD-1 pathway inhibitor.

In some aspects, a tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof. In certain aspects, the breast cancer is a triple negative breast cancer (TNBC). In certain aspects, the brain cancer is a glioblastoma. In some aspects, the skin cancer is a basal cell carcinoma (BCC), cutaneous squamous cell carcinoma (cSCC), melanoma, Merkel cell carcinoma (MCC), or a combination thereof. In further aspects, the head and neck cancer is a head and neck squamous cell carcinoma. In some aspects, the lung cancer is a small cell lung cancer (SCLC). In certain aspects, the esophageal cancer is gastroesophageal junction cancer. In some aspects, the kidney cancer is renal cell carcinoma. In some aspects, the liver cancer is hepatocellular carcinoma.

In some aspects, an IL-7 protein is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

In some aspects, a PD-1 pathway inhibitor is administered to the subject parenthetically, intramuscularly, subcutaneously, intravenously, or intraperitoneally.

Also provided herein are methods of treating a tumor in a human subject in need thereof, comprising administering to the subject an effective amount of an interleukin-7 (IL-7) protein in combination with an effective amount of a CTLA-4 pathway inhibitor. In certain aspects, a tumor volume is decreased by at least about 5%, at least about 10%, at least about 20%, at least about 30%, at least about 40%, at least about 50%, at least about 60%, at least about 70%, at least about 80%, at least about 90%, or at least about 100% after the administration. In some aspects, the human subject exhibits a lymphopenia prior to the administration.

In some aspects, the CTLA-4 pathway inhibitor comprises an anti-CTLA-4 antibody. In certain aspects, the anti-CTLA-4 antibody comprises ipilimumab, tremelimumab (ticilimumab; CP-675,206), AGEN-1884, or combinations thereof.

In some aspects, the IL-7 protein and the CTLA-4 pathway inhibitor are administered concurrently. In other aspects, the IL-7 protein and the CTLA-4 pathway inhibitor are administered sequentially. In certain aspects, the IL-7 protein is administered to the subject prior to administering the CTLA-4 pathway inhibitor.

In some aspects, the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

In some aspects, the IL-7 protein of the present disclosure is administered at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, the IL-7 protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, the IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

In some aspects, the IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

In some aspects, the IL-7 protein is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks.

In some aspects, the IL-7 protein is administered parenthetically. In some aspects, the IL-7 protein is administered intravenously.

In some aspects, the IL-7 protein, the PD-1 pathway inhibitor, and/or the CTLA-4 pathway inhibitor are formulated in a composition comprising a bulking agent, stabilizing agent, surfactant, buffering agent, or combinations thereof.

In some aspects, the PD-1 pathway inhibitor is nivolumab and the composition comprises (a) a mannitol (e.g., about 30 mg), (b) pentetic acid (e.g., about 0.008 mg), (c) polysorbate 80 (e.g., about 0.2 mg), (d) sodium chloride (e.g., about 2.92 mg), and (e) sodium citrate dehydrate (e.g., about 5.88 mg). In certain aspects, the PD-1 pathway inhibitor is administered to the subject at a flat dose of about 240 mg every two weeks or about 480 mg every four weeks. In some aspects, the PD-1 pathway inhibitor is administered to the subject at a weight-based dose of about 3 mg/kg every two weeks.

In some aspects, the PD-1 pathway inhibitor is pembrolizumab and the composition comprises (a) a L-histidine (e.g., about 1.55 mg), (b) polysorbate 80 (e.g., about 0.2 mg), and (c) sucrose (e.g., about 70 mg). In certain aspects, the PD-1 pathway inhibitor is administered to the subject at a flat dose of about 200 mg every three weeks. In further aspects, the PD-1 pathway inhibitor is administered to the subject at a weight-based dose of about 2 mg/kg every three weeks.

In some aspects, the PD-1 pathway inhibitor is atezolizumab and the composition comprises (a) a glacial acetic acid (e.g., about 16.5 mg), (b) L-histidine (e.g., about 62 mg), (c) sucrose (e.g., about 821.6 mg), and (d) polysorbate 20 (e.g., about 8 mg). In certain aspects, the PD-1 pathway inhibitor is administered to the subject at a flat dose of about 1200 mg every three weeks.

In some aspects, the PD-1 pathway inhibitor is durvalumab and the composition comprises (a) a L-histidine (e.g., about 2 mg), (b) L-histidine hydrochloride monohydrate (e.g., about 2.7 mg), (c) a,a-trehalose dihydrate (e.g., about 104 mg), and (d) polysorbate 80 (e.g., about 0.2 mg). In certain aspects, the PD-1 pathway inhibitor is administered to the subject at a weight-based dose of about 10 mg/kg every two weeks.

In some aspects, the PD-1 pathway inhibitor is avelumab and the composition comprises (a) D-mannitol (e.g., about 51 mg), (b) glacial acetic acid (e.g., about 0.6 mg), (c) polysorbate 20 (e.g., about 0.5 mg), and (d) sodium hydroxide (e.g., about 0.3 mg). In some aspects, the PD-1 pathway inhibitor is administered to the subject at a flat dose of about 800 mg every two weeks.

In some aspects, the CTLA-4 pathway inhibitor is ipilimumab and the composition comprises (a) diethylene triamine pentaacetic acid (DTPA) (e.g., about 0.04 mg), (b) mannitol (e.g., about 10 mg), (c) polysorbate 80 (vegetable origin) (e.g., about 0.1 mg), (d) sodium chloride (e.g., about 5.85 mg), and (e) tris hydrochloride (e.g., about 3.15 mg). In certain aspects, the CTLA-4 pathway inhibitor is administered to the subject at a weight-based dose of about 3 mg/kg every three weeks. In further aspects, the CTLA-4 pathway inhibitor is administered to the subject at a weight-based dose of about 10 mg/kg every three weeks for four doses, followed by 10 mg/kg every twelve weeks.

In some embodiments, the IL-7 protein disclosed herein is formulated in a composition comprising (a) sodium citrate (e.g., about 20 mM), (b) sucrose (e.g., about 5%), (c) sorbitol (e.g., about 1.5%), and (d) Tween 80 (e.g., about 0.05%).

### BRIEF DESCRIPTION OF THE FIGURES

FIGS. 1A, 1B, and 1C show the effect of IL-7 protein and anti-PD-1 antibody administration on tumor volume in a mouse adenocarcinoma model. FIG. 1A provides a diagram of the schedule of tumor inoculation and treatment administration. FIGS. 1B and 1C provide comparison of tumor volume (mm³) in the different treatment groups from two separate studies, respectively. The treatment groups included: (1) IL-7-formulating buffer+isotype control antibody (circle); (2) IL-7 formulating buffer +anti-PD-1 antibody (triangle); (3) IL-7 protein +isotype control antibody (inverted triangle); and (4) IL-7 protein +anti-PD-1 antibody (diamond). The data are shown as mean ±S.E.M. All comparisons were performed using two-way ANOVA with Bonferroni posts-tests. “*” and “***” indicate a statistically significant difference (p<0.05 and p<0.0001, respectively) compared to the control animals.

FIGS. 2A, 2B, and 2C show the effect of IL-7 protein and anti-PD-1 antibody administration on the number of tumor-infiltrating lymphocytes (TILs) in the animals from different treatment groups. FIG. 2A provides a diagram of the schedule of tumor inoculation and treatment administration. FIG. 2B provides a comparison of the number of CD4⁺ TILs from the different treatment groups. FIG. 2C provides a comparison of the number of CD8⁺ TILs from the different treatment groups. The treatment groups included: (1) IL-7-formulating buffer +isotype control antibody; (2) IL-7-formulating buffer +anti-PD-1 antibody; (3) IL-7 protein +isotype control antibody; and (4) IL-7 protein +anti-PD-1 antibody. In both FIGS. 2B and 2C, the number of CD4⁺ TILs and the CD8⁺ TILs are shown as a percentage of total CD45⁺ cells within the tumors. The data are shown both for individual animals and as mean ±S.E.M. All comparisons were performed using one-way ANOVA with Tukey's multiple comparison test. “*,” “**,” and “***” indicate a statistically significant difference (p<0.05, p<0.01, and p<0.0001, respectively) compared to the control animals.

FIGS. 3A, 3B, and 3C show the effect of triple combination of cyclophosphamide (CPA), IL-7 protein, and PD-1 pathway inhibitor on tumor volume and survival in the animals from the different treatment groups. FIG. 3A provides a diagram of the schedule of tumor inoculation and treatment administration. FIG. 3B provides comparison of tumor volume (mm³) in the different treatment groups at various time points post CPA treatment. FIG. 3C provides the survival data. The treatment groups included: (1) PBS+IL-7-formulating buffer+isotype control antibody; (2) CPA +IL-7-formulating buffer+isotype control antibody; (3) CPA+IL-7 protein +isotype control antibody; (4) CPA+IL-7 protein +anti-PD-1 antibody; and (5) CPA+IL-7 protein+anti-PD-L1 antibody. In FIG. 3B, the data are shown as mean ±S.E.M. Comparison of the different treatment groups were performed using two-way ANOVA with Bonferroni post-tests. “*” and “***” indicate a statistically significant difference (p<0.05 and p<0.001, respectively) compared to the control animals.

FIGS. 4A and 4B show the effect of IL-7 protein and anti-PD-1 antibody administration on tumor volume in thymectomized animals. FIG. 4A provides a diagram of the study design. FIG. 4B provides a comparison of tumor volume (mm³) in the different treatment groups. The treatment groups included: (1) IL-7-formulating buffer+isotype control antibody (circle); (2) IL-7 protein+isotype control antibody (square); (3) IL-7-formulating buffer+anti-PD-1 antibody (triangle); and (4) IL-7 protein+anti-PD-1 antibody (inverted triangle). The arrows indicate when IL-7 protein (gray arrow) and anti-PD-1 antibody (black arrows) were administered. The data are shown as mean ±S.E.M. Comparison of the different treatment groups were performed using two-way ANOVA with Bonferroni post-tests. “***” indicates a statistically significant difference (p<0.001) compared to the control animals.

FIGS. 5A, 5B, and 5C show the effect of IL-7 protein and anti-PD-1 antibody administration on the number of tumor-infiltrating lymphocytes (TILs) in thymectomized animals. FIG. 5A provides a diagram of the study design. FIGS. 5B and 5C provide comparison of the number of CD4⁺ TILs and CD8⁺ TILs, respectively. The treatment groups included: (1) IL-7-formulating buffer+isotype control antibody (“control”); (2) IL-7-formulating buffer+anti-PD-1 antibody (“a-PD1”); (3) IL-7 protein+isotype control antibody (“IL-7”); and (4) IL-7 protein+anti-PD-1 antibody (“Combo”). In both FIGS. 5B and 5C, the number of CD4⁺ and CD8⁺ TILs are shown as percentage of total CD45⁺ cells in the tumors. The data are shown both for individual animals and as mean ±S.E.M. All comparisons were performed using one-way ANOVA with Tukey's multiple comparison test. “*,” “**,” and “***” indicate a statistically significant difference (p<0.05, p<0.01, and p<0.0001, respectively) compared to the control animals.

FIGS. 6A and 6B show the effect of IL-7 protein on cytokine-induced T cell proliferation and activation in normal C57BL/6 mice. FIG. 6A show the kinetics of CD8+ T cell subsets in the blood after treatment with the IL-7 protein. The CD8+ T cell subsets shown include: (i) total CD8+ T cells (left graphs), (ii) CD8+ CD44-cells (middle graphs), and (iii) CD8+ CD44+ cells (right graphs). The top row shows the number of CD8+ T cell subsets as a percentage of total leukocytes. The bottom row shows the percentage of CD8+ T cell subsets that are Ki67+ (i.e., actively proliferating). Control animals received buffer alone (open circle). The data are shown as mean ±S.D. FIG. 6B shows the expression profile (blue line) of different activation markers on CD8+ splenic T cells at day 5 post IL-7 protein administration. The black line corresponds to the isotype control. The activation markers shown include (from left to right): T-bet, Eomes, PD-1, Granzyme B (GzmB), CXCR3, IFN-γ, TNF-α, and IL-2.

FIGS. 7A, 7B, and 7C show the effect of IL-7 protein administration on the activation and proliferation of naïve (top row) and central memory CD8+ T cells (bottom row) in mice. FIG. 7A shows CD44 and CD62L expression profile of the naïve and central memory splenic T cells 5 days after IL-7 administration. FIGS. 7B and 7C provide the proliferation data (based on CTV staining and Ki67 expression, respectively). In FIGS. 7B and 7C, blue represents T cells from animals that received the IL-7 protein, whereas orange represents T cells from the control animals (i.e., received buffer alone).

FIGS. 8A, 8B, and 8C show the dose-dependent anti-tumor effect of IL-7 protein administration in a syngeneic tumor model. FIG. 8A provides comparison of tumor volume (mm³) in animals that received different concentrations of the IL-7 protein: (i) 0 mg/kg (i.e., buffer alone) (black); (ii) 1.25 mg/kg (orange); (iii) 2.5 mg/kg (green); (iv) 5 mg/kg (blue); (v) 10 mg/kg (red). FIG. 8B provides the percentages of immune cell compartments among CD45+ cells from PBMCs at day 7 post IL-7 protein administration. The different immune cell compartments shown include: (i) CD8+ T cells (blue), (ii) CD4+ T cells (orange), (iii) Foxp3+ CD4+ regulatory T cells (purple), (iv) B220+ B cells (gray), and (v) other immune cells that do not fall within any of the earlier four categories (white). Each column represents a different concentration of IL-7 protein. FIG. 8C shows the absolute number of different immune cell populations in PBMCs at day 7 after treatment. The immune cell populations shown include: (i) CD8+ T cells (first graph), (ii) CD4+ T cells (second graph), (iii) Foxp3+ regulatory T cells (third graph), and (iv) B220+ B cells (fourth graph). The x-axis provides the concentrations of the IL-7 protein that were administered to the different treatment groups. *p<0.05, **p<0.01, ***p<0.001 versus Buffer group by 2-way ANOVA with Bonferroni post-tests (FIG. 8A), or 1-way ANOVA with Dunnett post-tests (FIG. 8C). Data are presented as mean ±S.D.

FIGS. 9A, 9B, 9C, 9D, 9E, 9F, 9G, and 9H show that IL-7 protein can confer antitumor activity by inducing a CD8+ T-cell inflamed tumor microenvironment. FIG. 9A provides the percentage (top graph) and the number (bottom graph) of different tumor-infiltrating leukocytes (TILs) observed in the tumors of mice at 5 days post IL-7 protein administration (purple) or buffer alone (orange). The different TILs shown include (i) monocytic myeloid-derived suppressor cells (M-MDSCs), (ii) polymorphonuclear myeloid-derived suppressor cells (PMN-MDSCs), (iii) tumor-associated macrophages (TAMs), (iv) tumor-associated dendritic cells (TADCs), (v) CD8+ T cells, (vi) CD4+ T helper cells (CD4 Th cells), (v) CD4+ regulatory T cells (Treg cells), (vi) NK cells, and (vii) B cells. FIG. 9B provides a comparison of the ratio of CD8+ TILs to Foxp3+ regulatory T cells (left graph) or MDSCs (right graph) in animals treated with buffer alone (orange) or IL-7 protein (purple) at 5 days post-treatment. FIG. 9C provides a comparison of the percentage of Ki67+ (left graph) or granzyme B+ (right graph) cells among CD8+ TILs in animals treated with buffer alone (orange) or IL-7 protein (purple) at 5 days post-treatment. FIG. 9D provides the percentage of IFN-γ and/or TNF-α producing CD8+ TILs in animals treated with buffer alone (orange) or IL-7 protein (purple) at 5 days post-treatment. FIG. 9E shows the percentage of PD-1+ cells among CD8+ TILs in animals treated with buffer alone or IL-7 protein. FIG. 9F shows the percentage of LAG-3+ TIM-3+ cells among CD8+ PD-1+ TILs. FIG. 9G shows the geometric mean fluorescence intensity (gMFI) of the expression level of different immune checkpoint receptors on PD-1+LAG-3+TIM-3+CD8+TILs in animals treated with buffer alone or IL-7 protein. FIG. 9H provides the relative expression of different chemokines (CCL2, CCLS, CXCL1, CXCL9, CXCL10, and CXCL11) measured in tumor lysates as measured by RT-qPCR from animals treated with buffer (white) or IL-7 protein (green). In each of FIGS. 9A-9H, *p<0.05, **p<0.01, ***p<0.001 versus Buffer group by unpaired t-test. Data presented as mean ±s.d.

FIG. 10 shows the anti-tumor effects of IL-7 protein in combination with cyclophosphamide (CPA) and/or immune checkpoint inhibitors. The graphs to the left provides the tumor volume (mm³) at different time points after treatment. The graphs to the right provides the survival data. The top row provides results for animals treated with (i) buffer alone, (ii) combination of CPA and anti-PD-1 antibody, or (iii) combination of CPA, anti-PD-1 antibody, and IL-7 protein. The middle row provides results for animals treated with (i) buffer alone, (ii) combination of CPA and anti-PD-L1 antibody, or (iii) combination of CPA, anti-PD-L1 antibody, and IL-7 protein. The bottom row provides results for animals treated with (i) buffer alone, (ii) combination of CPA and anti-CTLA-4 antibody, or (iii) combination of CPA, anti-CTLA-4 antibody, and IL-7 protein. **p<0.01, ***p<0.001 versus the corresponding color group in the legend by Log-rank (Mantel-Cox) test. Data presented as mean ±s.d.

FIG. 11A provides a comparison of CD8+ T cell numbers in the spleen, peripheral blood, and lymph nodes of thymectomized animals and sham controls. FIG. 11B shows the number of different CD8+ T cell population in the spleen of tumor mice treated with PBS or IL-7 protein at various weeks after administration. The CD8+ T cell populations shown include: (i) total CD8+ T cells (first graph); (ii) naïve (CD44− CD62L+) CD8+ T cells (second graph), (iii) effector memory (CD44+ CD62L−) CD8+ T cells (third graph), and (iv) central memory (CD44+ CD62L+) CD8+ T cells (fourth graph). *p<0.05, **p<0.01, ***p<0.001 between the indicated groups by unpaired t-test.

FIG. 12 provides a schematic of the study design for the phase lb clinical trial described in Example 11 assessing the safety and efficacy of IL-7 protein in patients with advanced solid cancer.

FIG. 13 provides a table summarizing the adverse effects observed in advanced solid cancer patients from the phase lb clinical trial described in Example 11. “TEAE” refers to any treatment emergent adverse events. “ADR” refers to adverse drug reaction.

FIGS. 14A, 14B, and 14C provide the results of the pharmacokinetic analysis from the phase 1b clinical trial described in Example 11. FIG. 14A provides a comparison of the IL-7 concentration in the serum of advanced solid cancer patients treated with different doses of IL-7 protein. As described in Example 11, the doses included the following: (i) 60 μg/kg (“1”), (ii) 120 μg/kg (“2”), (iii) 240 μg/kg (“3”), (iv) 480 μg/kg (“4”), (v) 720 μg/kg (“5”), (vi) 960 μg/kg (“6”), and (vii) 1,200 μg/kg (“7”). FIGS. 14B and 14C provide comparison of the Cₘₐₓ and AUC in advanced solid cancer patients from the dosage groups. Data are shown as mean±SEM for each dose level.

FIGS. 15A, 15B, 15C, 15D, 15E, and 15F provide the results of the pharmacodynamics analysis from the phase 1b clinical trial described in Example 11. FIGS. 15A to 15D provide a comparison of the absolute lymphocyte count (ALC), CD3+, CD4+, and CD8+ T cell numbers, respectively, in advanced solid cancer patients at prior to IL-7 protein administration (i.e., time “0”) and at three weeks post 1^{st} dose. FIGS. 15E and 15F provide a comparison of ALC in non-lymphopenic and lymphopenic patients, respectively. In each of the figures, the patients were categorized into low (60 and 120 μg/kg) (“circle”), medium (240 and 480 μg/kg) (“square”), and high dose groups (720 and 1,200 μg/kg) (“triangle”). “*” refers to p<0.05, “**” refers to p<0.01, and “***” refers to p<0.001 versus baseline (0 week) group by Wilcoxon matched-pairs signed rank test.

FIGS. 16A, 16B, 16C, 16D, 16E, 16F, 16G, and 16H provide a comparison of the effect of IL-7 protein administration on different CD4+ and CD8+ T cell subsets in patients from the phase 1b clinical trial described in Example 11. FIGS. 16A and 16C provide a comparison of Ki67+ CD4+ and Ki67+ CD8+ T cells, respectively, in the patients prior to IL-7 protein administration (i.e., time “0”) and at week one post administration (i.e., time “1”). FIGS. 16B and 16D provide a comparison of CD127+ CD4+ and CD127+ CD8+ T cells, respectively, in the patients prior to IL-7 protein administration (i.e., time “0”) and at week one post administration (i.e., time “1”). FIG. 16E provide a comparison of the CD4+ T cell/Treg ratio (left graph) and CD8+ T cell/Treg ratio (right graph) in the patients prior to IL-7 protein administration (i.e., time “0”) and at week one post administration (i.e., time “1”). FIG. 16F provide a comparison of naïve (left column), effector memory (EM) (middle column), and central memory (CM) (right column) subsets for both CD4+ T cells (top row) and CD8+ T cells (bottom row) in patients prior to IL-7 protein administration (i.e., time “0”) and at week three post administration (i.e., time “3”). FIGS. 16G and 16H provide a comparison of CCR5+ CD4+ and CCR5+ CD8+ T cells, respectively, in the patients prior to IL-7 protein administration (i.e., time “0”) and at week one post administration (i.e., time “1”). In each of the figures, the patients were categorized into low (60 and 120 μg/kg) (circle; left two columns), medium (240 and 480 μg/kg) (square; middle two columns), and high dose groups (720 and 1,200 μg/kg) (triangle; right two columns). “*” refers to p<0.05, “*” refers to p<0.01, and “***” refers to p<0.001 versus baseline (0 week) group by Wilcoxon matched-pairs signed rank test.

FIGS. 17A and 17B provide a comparison of the effect of IL-7 protein administration on NK and B cells, respectively in the patients from the phase lb clinical trial described in Example 11, prior to IL-7 protein administration (i.e., time “0”) and at week three post administration (i.e., time “3”). In both of the figures, the patients were categorized into low (60 and 120 μg/kg) (circle; left two columns), medium (240 and 480 μg/kg) (square; middle two columns), and high dose groups (720 and 1,200 μg/kg) (triangle; right two columns). “*” refers to p<0.05 versus baseline (0 week) group by Wilcoxon matched-pairs signed rank test.

FIG. 18 provides a table summarizing the adverse effects observed in glioblastoma patients from the phase lb clinical trial described in Example 12. “TEAE” refers to any treatment emergent adverse events. “ADR” refers to adverse drug reaction.

FIGS. 19A, 19B, 19C, 19D, 19E, and 19F provide the results of the pharmacodynamics analysis from the phase 1b clinical trial described in Example 12. FIGS. 19A to 19D provide a comparison of the absolute lymphocyte count (ALC), CD3+, CD4+, and CD8+ T cell numbers, respectively, in glioblastoma cancer patients, prior to IL-7 protein administration (i.e., time “0”) and at three weeks post 1^{st} dose. FIGS. 19E and 19F provide a comparison of ALC in non-lymphopenic and lymphopenic patients, respectively. In each of the figures, the patients were categorized into low (60 μg/kg) (circle), medium (360 and 600 μg/kg) (square), and high dose groups (840 and 1,440 μg/kg) (triangle). “*” refers to p<0.05 and “**” refers to p<0.01 versus baseline (0 week) group by Wilcoxon matched-pairs signed rank test.

FIGS. 20A, 20B, and 20C show the effect of IL-7 protein administration on AUC, Ki67+ CD8+ T cell frequency, and Ki67+ CD4+ T cell frequency, respectively, in glioblastoma patients receiving temozolomide (TMZ). In each of the figures, days on which TMZ or IL-7 protein were administered are indicated.

FIGS. 21A, 21B, and 21C show the effect of IL-7 protein administration on AUC, Ki67+ CD8+ T cell frequency, and Ki67+ CD4+ T cell frequency, respectively, in glioblastoma patients receiving avastin/irinotecan (A/I). In each of the figures, days on which A/I or IL-7 protein were administered are indicated.

### DETAILED DESCRIPTION OF THE INVENTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, a therapeutic agent described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten.

The terms “antibody” and “antibodies” are terms of art and can be used interchangeably herein and refer to a molecule with an antigen binding site that specifically binds an antigen. The terms as used to herein include whole antibodies and any antigen binding fragments (i.e., “antigen-binding portions”) or single chains thereof. An “antibody” refers, in one aspect, to a glycoprotein comprising at least two heavy (H) chains and two light (L) chains inter-connected by disulfide bonds, or an antigen-binding portion thereof. In another aspect, an “antibody” refers to a single chain antibody comprising a single variable domain, e.g., VHH domain. Each heavy chain is comprised of a heavy chain variable region (abbreviated herein as VH) and a heavy chain constant region. In certain naturally-occurring antibodies, the heavy chain constant region is comprised of three domains, CH1, CH2 and CH3. In certain naturally-occurring antibodies, each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (K_{D}) of 10⁻⁵ to 10⁻¹¹M or less. Any K_{D} greater than about 10⁻⁴M is generally considered to indicate nonspecific binding. As used herein, an antibody that “binds specifically” to an antigen refers to an antibody that binds to the antigen and substantially identical antigens with high affinity, which means having a K_{D} of 10⁻⁷M or less, 10⁻⁸M or less, 5×10⁻⁹M or less, or between 10⁻⁸M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, at least 95%, at least 97%, or at least 99% sequence identity to the sequence of the given antigen. By way of example, an antibody that binds specifically to PD-1 can, in certain aspects, also have cross-reactivity with PD-1 antigens from certain primate species (e.g., cynomolgus anti-PD-1 antibody), but cannot cross-react with PD-1 molecules from other species or with a molecule other than PD-1.

An immunoglobulin can be derived from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. In certain aspects, one or more amino acids of the isotype can be mutated to alter effector function. The term “antibody” includes, by way of example, both naturally occurring and non-naturally occurring Abs; monoclonal and polyclonal Abs; chimeric and humanized Abs; human or nonhuman Abs; wholly synthetic Abs; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 can, however, have cross-reactivity to other antigens, such as PD-1 molecules from different species. Moreover, an isolated antibody can be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs can be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-PD-1 antibody binds specifically to PD-1 and an anti-CTLA-4 antibody binds specifically to CTLA-4.

An “antigen-binding portion” of an antibody (also called an “antigen-binding fragment”) refers to one or more fragments of an antibody that retain the ability to bind specifically to the antigen bound by the whole antibody.

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE™ 2000 instrument using the predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain aspects, a predicted nonessential amino acid residue in an antibody is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology= # of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, e.g., as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the) XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

“Cytotoxic T-Lymphocyte Antigen-4” (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

The term “fusion protein” refers to proteins created through the joining of two or more genes that originally coded for separate proteins. Translation of this fusion gene results in a single polypeptide or multiple polypeptides with functional properties derived from each of the original proteins. In some aspects, the two or more genes can comprise a substitution, a deletion, and/or an addition in its nucleotide sequence.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1: 1).

Additionally, an Fc (native or variant) of the present invention can be in the form of having native sugar chains, increased sugar chains, or decreased sugar chains compared to the native form, or may be in a deglycosylated form. The immunoglobulin Fc sugar chains can be modified by conventional methods such as a chemical method, an enzymatic method, and a genetic engineering method using a microorganism. The removal of sugar chains from an Fc fragment results in a sharp decrease in binding affinity to the C1q part of the first complement component C1, and a decrease or loss of ADCC or CDC, thereby not inducing any unnecessary immune responses in vivo. In this regard, an immunoglobulin Fc region in a deglycosylated or aglycosylated form may be more suitable to the object of the present invention as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell.

An “immunomodulator” or “immunoregulator” refers to an agent, e.g., a component of a signaling pathway, that can be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In preferred aspects, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Immunostimulating therapy” or “immunostimulatory therapy” refers to a therapy that results in increasing (inducing or enhancing) an immune response in a subject for, e.g., treating cancer.

“Potentiating an endogenous immune response” means increasing the effectiveness or potency of an existing immune response in a subject. This increase in effectiveness and potency can be achieved, for example, by overcoming mechanisms that suppress the endogenous host immune response or by stimulating mechanisms that enhance the endogenous host immune response.

The term “effector T cells” (Teff) refers to T cells (e.g., CD4⁺ and CD8⁺ T cells) with cytolytic activities as well as T helper (Th) cells, which secrete cytokines and activate and direct other immune cells, but does not include regulatory T cells (Treg cells). Combination of an IL-7 protein and an immune checkpoint inhibitor (e.g., an anti-PD-1 antibody) activate and/or increase the frequency of Teff cells, e.g., CD4⁺ and CD8⁺ T cells, in a tumor or blood of a subject.

As used herein, the term “regulatory T cells” (Tregs) refer to a population of T cells with the ability to reduce or suppress the induction and proliferation of effector T cells, and thereby, modulate an immune response. In some aspects, Tregs can suppress an immune response by secreting anti-inflammatory cytokines, such as IL-10, TGF-β, and IL-35, which can interfere with the activation and differentiation of naïve T cells into effector T cells. In some aspects, Tregs can also produce cytolytic molecules, such as Granzyme B, which can induce the apoptosis of effector T cells. In some aspects, the regulatory T cells are natural regulatory T cells (nTregs) (i.e., developed within the thymus). In some aspects, the regulatory T cells are induced regulatory T cells (iTregs) (i.e., naïve T cells that differentiate into Tregs in the peripheral tissue upon exposure to certain stimuli). Methods for identifying Tregs are known in the art. For example, Tregs express certain phenotypic markers (e.g., CD25, Foxp3, or CD39) that can be measured using flow cytometry. See, e.g., International Publication No. WO 2017/062035 A1; Gu J., et al., Cell Mol Immunol 14(6): 521-528 (2017). In some aspects, the Tregs are CD45RA⁻ CD39⁺ T cells.

As used herein, the term “tumor infiltrating lymphocytes” or “TILs” refers to lymphocytes (e.g., effector T cells) that have migrated from the periphery (e.g., from the blood) into a tumor. In some aspects, the tumor infiltrating lymphocytes are CD4+ TILs. In other aspects, the tumor infiltrating lymphocytes are CD8+ TILs.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system can be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

As used herein, the term “interleukin-7” or “IL-7” refers to IL-7 polypeptides and derivatives and analogs thereof having substantial amino acid sequence identity to wild-type mature mammalian IL-7s and substantially equivalent biological activity, e.g., in standard bioassays or assays of IL-7 receptor binding affinity. For example, IL-7 refers to an amino acid sequence of a recombinant or non-recombinant polypeptide having an amino acid sequence of: i) a native or naturally-occurring allelic variant of an IL-7 polypeptide, ii) a biologically active fragment of an IL-7 polypeptide, iii) a biologically active polypeptide analog of an IL-7 polypeptide, or iv) a biologically active variant of an IL-7 polypeptide. IL-7 polypeptides of the invention can be obtained from any species, e.g., human, cow or sheep. IL-7 nucleic acid and amino acid sequences are well known in the art. For example, the human IL-7 amino acid sequence has a Genbank accession number of P13232 (SEQ ID NO: 1); the mouse IL-7 amino acid sequence has a Genbank accession number of P10168 (SEQ ID NO: 3); the rat IL-7 amino acid sequence has a Genbank accession number of P56478 (SEQ ID NO: 2); the monkey IL-7 amino acid sequence has a Genbank accession number of NP 001279008 (SEQ ID NO: 4); the cow IL-7 amino acid sequence has a Genbank accession number of P26895 (SEQ ID NO: 5); and the sheep IL-7 amino acid sequence has a Genbank accession number of Q28540 (SEQ ID NO: 6). In some aspects, an IL-7 polypeptide of the present disclosure is a variant of an IL-7 protein.

A “variant” of an IL-7 protein is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological activity can be found using computer programs well known in the art, for example software for molecular modeling or for producing alignments. The variant IL-7 proteins included within the invention include IL-7 proteins that retain IL-7 activity. IL-7 polypeptides which also include additions, substitutions or deletions are also included within the invention as long as the proteins retain substantially equivalent biological IL-7 activity. For example, truncations of IL-7 which retain comparable biological activity as the full length form of the IL-7 protein are included within the invention. The activity of the IL-7 protein can be measured using in vitro cellular proliferation assays such as described in Example 6 below. The activity of IL-7 variants of the invention maintain biological activity of at least 10%, 20%, 40%, 60%, but more preferably 80%, 90%, 95% and even more preferably 99% as compared to wild type IL-7.

Variant IL-7 proteins also include polypeptides that have at least about 70%, 75%, 80%, 85%, 90%, 92%, 95%, 96%, 97%, 98%, 99%, or more sequence identity with wild-type IL-7. To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=# of identical positions/total # of positions.times.100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al., (1990) J. Mol. Biol. 215:403-10. BLAST nucleotide searches can be performed with the NBLAST program, score=100, wordlength=12. BLAST protein searches can be performed with the) XBLAST program, score=50, wordlength=3. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used.

As used herein, the term “Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

As used herein, the term “Programmed Death Ligand-1 (PD-L1)” refers to one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In some aspects, a “therapeutically effective amount” is the amount of IL-7 protein and the amount of an immune checkpoint inhibitor (e.g., PD-1 pathway inhibitor, e.g., anti-PD-1 antibody), in combination, clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “dosing frequency” refers to the number of times a therapeutic agent (e.g., an IL-7 protein or an immune checkpoint inhibitor) is administered to a subject within a specific time period. Dosing frequency can be indicated as the number of doses per a given time, for example, once per day, once a week, or once in two weeks. As used herein, “dosing frequency” is applicable where a subject receives multiple (or repeated) administrations of a therapeutic agent.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

As used herein, the term “drug” refers to any bioactive agent (e.g., an IL-7 protein or an immune checkpoint inhibitor) intended for administration to a human or non-human mammal to prevent or treat a disease or other undesirable condition. Drugs include hormones, growth factors, proteins, peptides and other compounds. In some aspects, a drug disclosed herein is an anti-cancer agent.

By way of example, an “anti-cancer agent” promotes cancer regression in a subject or prevents further tumor growth. In certain aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent can inhibit cell growth or tumor growth by at least about 10%, at least about 20%, by at least about 40%, by at least about 60%, or by at least about 80% relative to untreated subjects or, in certain aspects, relative to patients treated with a standard-of-care therapy. In other aspects of the invention, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The term “reference,” as used herein, refers to a corresponding subject (e.g., a cancer subject) who did not receive a combination of IL-7 protein and an immune checkpoint inhibitor, e.g., a subject who received IL-7 protein alone or immune checkpoint inhibitor alone. In some aspects, the reference subject received neither IL-7 protein nor immune checkpoint inhibitor. The term “reference” can also refer to a same cancer subject but prior to the administration of a combination of IL-7 protein and an immune checkpoint inhibitor. In certain aspects, the term “reference” refers to an average of a population of subjects (e.g., cancer subjects).

As used herein, the terms “ug” and “uM” are used interchangeably with “μg” and “μM,” respectively.

Various aspects described herein are described in further detail in the following subsections.

### II. Methods of the Disclosure

The present disclosure is directed to a method for treating a tumor (or a cancer) in a subject in need thereof, comprising administering to the subject an effective amount of an interleukin-7 (IL-7) protein in combination with an effective amount of an immune checkpoint inhibitor. Non-limiting examples of immune checkpoint inhibitors that can be used with the current methods include an anti-PD-1 antibody, anti-PD-L1 antibody, anti-CTLA-4 antibody, and combinations thereof.

In some aspects, a combination of IL-7 protein and an immune checkpoint inhibitor can increase the absolute lymphocyte count in a subject when administered to the subject. In certain aspects, the absolute lymphocyte count is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% or more, compared to a reference (e.g., value in a corresponding subject after administration of IL-7 protein alone or immune checkpoint inhibitor alone).

In some aspects, administering a combination disclosed herein (i.e., combination of IL-7 protein and an immune checkpoint inhibitor) to a subject can increase T cell proliferation (e.g., CD8⁺ T cells) in the subject. In certain aspects, the increase in T cell proliferation occurs in the periphery (e.g., not within the tumor). In some aspects, administering a combination of an IL-7 protein and an immune checkpoint inhibitor can increase the recruitment of effector T cells (e.g., cytotoxic CD8⁺ T lymphocytes) to the tumor in a subject.

In certain aspects, T cell proliferation is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% or more, compared to a reference (e.g., value in a corresponding subject after administration of IL-7 protein alone or immune checkpoint inhibitor alone). In certain aspects, T cells (e.g., CD8⁺ T cells) that proliferate in response to the IL-7 administration express one or more of the following markers: Eomesodermin (Eomes), granzyme B, CXCR3, IFN-γ, or combinations thereof.

In certain aspects, recruitment of effector T cells to the tumor is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% or more, compared to a reference (e.g., value in a corresponding subject after administration of IL-7 protein alone or immune checkpoint inhibitor alone).

In some aspects, administering a combination of IL-7 protein and an immune checkpoint inhibitor inhibits and/or reduces tumor growth in a subject. In some aspects, the tumor growth (e.g., tumor volume or weight) is reduced by at least about 5%, at least about 10%, at least about 20%, at least about 30%, at least about 40%, at least about 50%, at least about 60%, at least about 70%, at least about 80%, at least about 90%, or about 100% compared to a reference (e.g., tumor volume in a corresponding subject after administration of IL-7 protein alone or immune checkpoint inhibitor alone).

In some aspects, administering a combination of an IL-7 protein and an immune checkpoint inhibitor treats a tumor or a subject afflicted with a tumor by promoting and/or enhancing an immune response against a tumor antigen. In some aspects, administering a composition of the present disclosure increases the number and/or percentage of tumor-infiltrating lymphocytes (TILs) (e.g., CD4⁺ or CD8⁺) in a tumor of a subject. In some aspects, the number and/or percentage of TILs is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration compared to a reference (e.g., number and/or percentage of TILs in a tumor of a subject treated with either IL-7 protein alone or an immune checkpoint inhibitor alone).

In some aspects, administering a combination of an IL-7 protein and an immune checkpoint inhibitor reduces the number and/or percentage of regulatory T cells (Tregs) in a tumor of a subject. In some aspects, the regulatory T cells are CD4⁺ regulatory T cells. In some aspects, the regulatory T cells are Foxp3⁺. In certain aspects, the number and/or percentage of regulatory T cells in a tumor is decreased by at least about 5%, at least about 10%, at least about 20%, at least about 30%, at least about 40%, at least about 50%, at least about 60%, at least about 70%, at least about 80%, at least about 90%, or about 100% compared to a reference (e.g., the corresponding number and/or percentage in a subject that received IL-7 protein alone or an immune checkpoint inhibitor alone).

In some aspects, administering a combination of an IL-7 protein and an immune checkpoint inhibitor increases the ratio of CD8⁺ TILs to Tregs in a tumor of a subject. In certain aspects, the ratio of CD8⁺ TILs to Tregs is increased by at least about 5%, at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration compared to a reference (e.g., number and/or percentage of TILs in a tumor of a subject treated with either IL-7 protein alone or an immune checkpoint inhibitor alone).

In some aspects, administering a combination of an IL-7 protein and an immune checkpoint inhibitor decreases the number and/or percentage of myeloid-derived suppressor cells (MDSCs) in the tumor of a subject. As used herein, the term “myeloid-derived suppressor cells” (MDSCs) refer to a heterogeneous population of immune cells that are defined by their myeloid origin, immature state, and ability to potently suppress T cell responses. They are known to expand in certain pathological conditions, such as chronic infections and cancers. In certain aspects, the MDSCs are monocytic MDSCs (M-MDSCs). In other aspects, the MDSCs are polymorphonuclear MDSCs (PMN-MDSCs). In some aspects, the number and/or percentage of MDSCs in the tumor is decreased by at least about 5%, at least about 10%, at least about 20%, at least about 30%, at least about 40%, at least about 50%, at least about 60%, at least about 70%, at least about 80%, at least about 90%, or about 100% compared to a reference (e.g., value in a corresponding subject after administration of IL-7 protein alone or immune checkpoint inhibitor alone).

In some aspects, administering a combination of an IL-7 protein and an immune checkpoint inhibitor increases the ratio of CD8⁺ TILs to MDSCs in a tumor of a subject. In certain aspects, the ratio of CD8⁺ TILs to MDSCs is increased by at least about 5%, at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration compared to a reference (e.g., value in a corresponding subject after administration of IL-7 protein alone or immune checkpoint inhibitor alone).

In some aspects, administering a combination of an IL-7 protein and an immune checkpoint inhibitor reduces the expression of an immune checkpoint inhibitor molecule (e.g., PD-1) on TILs in a subject. In certain aspects, a combination of an IL-7 protein and an immune checkpoint inhibitor reduces the mean fluorescence index (MFI) of immune checkpoint inhibitor molecule (e.g., PD-1) expression on TILs. In some aspects, the immune checkpoint inhibitor molecule is PD-1. In certain aspects, administering a combination of an IL-7 protein and an immune checkpoint inhibitor reduces the MFI of PD-1 expression on CD8⁺ TILs by at least about 5%, at least about 10%, at least about 20%, at least about 30%, at least about 40%, at least about 50%, at least about 60%, at least about 70%, at least about 80%, at least about 90%, or about 100% compared to a reference (e.g., the corresponding number and/or percentage in a subject that received IL-7 protein alone or an immune checkpoint inhibitor alone).

In some aspects, administering a combination of an IL-7 protein and an immune checkpoint inhibitor increases the expression of markers associated with effector (e.g., anti-tumor) activity on TILs in a subject. Non-limiting examples of markers associated with effector activity includes Ki-67, granzyme B, T-bet, Eomes, CXCR3, IFN-γ, TNF-α, and IL-2. In certain aspects, markers associated with effector activity comprises Ki-67 and granzyme B. In certain aspects, a combination of an IL-7 protein and an immune checkpoint inhibitor increases the mean fluorescence index (MFI) of the expression of a marker associated with effector activity on TILs by at least about 5%, at least about 10%, at least about 20%, at least about 30%, at least about 40%, at least about 50%, at least about 60%, at least about 70%, at least about 80%, at least about 90%, or about 100% compared to a reference, (e.g., corresponding value in a subject that did not receive a combination of an IL-7 protein and an immune checkpoint inhibitor).

As described supra, many cancer patients are lymphopenic, as many of the available standard of care cancer treatments (e.g., chemotherapy and radiation therapy) are known to cause lymphopenia. Accordingly, methods disclosed herein can also be used to treat a cancer in a lymphopenic subject.

As used herein, the term “lymphopenic subject” refers to a subject with lymphopenia. As used herein, the terms “lymphopenia” and “lymphocytopenia” are used interchangeably and refer to a condition characterized by abnormally low number of circulating immune cells (e.g., lymphocytes). Peripheral circulation of all types of lymphocytes or subpopulations of lymphocytes (e.g., CD4⁺ T cells) can be depleted or abnormally low in a patient suffering from lymphopenia. See, e.g., Lymphopenia Description, The Merck Manual (18th Edition, 2006, Merck & Co.). In some aspects, compared to a normal subject (e.g., healthy individual), a lymphopenic subject has reduced number of T-lymphocytes (“T-lymphopenia”), B-lymphocytes (“B-lymphopenia”), and/or NK cells (“NK lymphopenia”).

Quantitatively, lymphopenia can be described by various cutoffs. In some aspects, a lymphopenic subject has a circulating blood total lymphocyte count that is less than by at least about 5%, at least about 10%, at least about 20%, at least about 30%, at least about 40%, at least about 50%, at least about 60%, at least about 70%, at least about 80%, at least about 90%, or at least about 100% compared to a circulating blood total lymphocyte count in a corresponding subject who does not exhibit a lymphopenia. In some aspects, a subject has lymphopenia if the subject has a circulating blood total lymphocyte count of less than about 1,500 lymphocytes/μL, less than about 1,000 lymphocytes/μL, less than about 800 lymphocytes/μL, less than about 500 lymphocytes/μL, or less than about 200 lymphocytes/μL.

Lymphocytopenia has a wide range of possible causes. In some aspects, a lymphopenia is caused by or associated with a tumor. In some aspects, a lymphopenia is caused by or associated with a previous therapy for a tumor (e.g., chemotherapy or radiation therapy). In some aspects, a lymphopenia is caused by or associated with an infection, including viral (e.g., HIV or hepatitis infection), bacterial (e.g., active tuberculosis infection), and fungal infections; chronic failure of the right ventricle of the heart, Hodgkin's disease and cancers of the lymphatic system, leukemia, a leak or rupture in the thoracic duct, side effects of prescription medications including anticancer agents, antiviral agents, and glucocorticoids, malnutrition resulting from diets that are low in protein, radiation therapy, uremia, autoimmune disorders, immune deficiency syndromes, high stress levels, and trauma.

In some aspects, a lymphopenia is idiopathic (i.e., has unknown etiology). Non-limiting examples of idiopathic lymphopenia include idiopathic CD4 positive T-lymphocytopenia (ICL), acute radiation syndrome (ARS), or a combination thereof.

In some aspects, administering an IL-7 protein in combination with an immune checkpoint inhibitor to a lymphopenic subject with a tumor inhibits and/or reduces tumor growth in a subject. In some aspects, the tumor growth (e.g., tumor volume or weight) is reduced by at least about 5%, at least about 10%, at least about 20%, at least about 30%, at least about 40%, at least about 50%, at least about 60%, at least about 70%, at least about 80%, at least about 90%, or about 100% compared to a reference (e.g., tumor volume in a corresponding subject after administration of IL-7 protein alone or immune checkpoint inhibitor alone).

In some aspects, administering an IL-7 protein in combination with an immune checkpoint inhibitor to a lymphopenic subject with a tumor increases the number and/or percentage of tumor-infiltrating lymphocytes (TILs) (e.g., CD4⁺ or CD8⁺ ) in a tumor of a subject. In some aspects, the number and/or percentage of TILs is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration compared to a reference (e.g., number and/or percentage of TILs in a tumor of a subject treated with either IL-7 protein alone or an immune checkpoint inhibitor alone).

In some aspects, administering an IL-7 protein in combination with an immune checkpoint inhibitor to a lymphopenic subject with a tumor reduces the number and/or percentage of regulatory T cells in a tumor of a subject. In some aspects, the regulatory T cells are Foxp3⁺. In certain aspects, the number and/or percentage of regulatory T cells in a tumor is decreased by at least about 5%, at least about 10%, at least about 20%, at least about 30%, at least about 40%, at least about 50%, at least about 60%, at least about 70%, at least about 80%, at least about 90%, or about 100% compared to a reference (e.g., the corresponding number and/or percentage in a subject that received IL-7 protein alone or an immune checkpoint inhibitor alone).

In some aspects, administering a combination of an IL-7 protein and an immune checkpoint inhibitor to a lymphopenic subject increases the ratio of CD8⁺ TILs to Tregs in a tumor of the subject. In certain aspects, the ratio of CD8⁺ TILs to Tregs is increased by at least about 5%, at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration compared to a reference (e.g., number and/or percentage of TILs in a tumor of a subject treated with either IL-7 protein alone or an immune checkpoint inhibitor alone).

In some aspects, administering a combination of an IL-7 protein and an immune checkpoint inhibitor to a lymphopenic subject reduces the expression of an immune checkpoint inhibitor molecule (e.g., PD-1) on TILs in a subject. In certain aspects, a combination of an IL-7 protein and an immune checkpoint inhibitor reduces the mean fluorescence index (MFI) of immune checkpoint inhibitor molecule (e.g., PD-1) expression on TILs. In some aspects, the immune checkpoint inhibitor molecule is PD-1. In certain aspects, administering a combination of an IL-7 protein and an immune checkpoint inhibitor reduces the MFI of PD-1 expression on CD8⁺ TILs by at least about 5%, at least about 10%, at least about 20%, at least about 30%, at least about 40%, at least about 50%, at least about 60%, at least about 70%, at least about 80%, at least about 90%, or about 100% compared to a reference (e.g., the corresponding number and/or percentage in a subject that received IL-7 protein alone or an immune checkpoint inhibitor alone).

Non-limiting examples of cancers (or tumors) that can be treated with methods disclosed herein include squamous cell carcinoma, small-cell lung cancer (SCLC), non-small cell lung cancer, squamous non-small cell lung cancer (NSCLC), nonsquamous NSCLC, gastrointestinal cancer, renal cancer (e.g., clear cell carcinoma), ovarian cancer, liver cancer (e.g., hepatocellular carcinoma), colorectal cancer, endometrial cancer, kidney cancer (e.g., renal cell carcinoma (RCC)), prostate cancer (e.g., hormone refractory prostate adenocarcinoma), thyroid cancer, pancreatic cancer, cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus (e.g., gastroesophageal junction cancer), cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, tumor angiogenesis, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CIVIL), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki1⁺) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, and any combinations thereof

In some aspects, a cancer (or tumor) that can be treated comprises a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, carcinoma, sarcoma, leukemia, lymphoma, myeloma, or a combination thereof. In certain aspects, a cancer (or tumor) that can be treated with the present methods is breast cancer. In some aspects, breast cancer is a triple negative breast cancer (TNBC). In some aspects, a cancer (or tumor) that can be treated is a brain cancer. In certain aspects, brain cancer is a glioblastoma. In some aspects, a cancer (or tumor) that can be treated with the present methods is skin cancer. In some aspects, skin cancer is a basal cell carcinoma (BCC), cutaneous squamous cell carcinoma (cSCC), melanoma, Merkel cell carcinoma (MCC), or a combination thereof. In certain aspects, a head and neck cancer is a head and neck squamous cell carcinoma. In further aspects, a lung cancer is a small cell lung cancer (SCLC). In some aspects, an esophageal cancer is gastroesophageal junction cancer. In certain aspects, a kidney cancer is renal cell carcinoma. In some aspects, a liver cancer is hepatocellular carcinoma.

In some aspects, the methods described herein can also be used for treatment of metastatic cancers, unresectable, refractory cancers (e.g., cancers refractory to previous cancer therapy, e.g., immunotherapy, e.g., with a blocking anti-PD-1 antibody), and/or recurrent cancers. In certain aspects, the previous cancer therapy comprises a chemotherapy. In some aspects, the chemotherapy comprises a platinum-based therapy. In some aspects, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain aspects, the platinum-based therapy comprises cisplatin. In further aspects, the platinum-based therapy comprises carboplatin.

In some aspects, a subject to be treated with the methods disclosed herein has received one, two, three, four, five or more prior cancer treatments. In other aspects, the subject is treatment-naïve (i.e., has never received a prior cancer treatment). In some aspects, the subject has progressed on other cancer treatments. In certain aspects, the prior cancer treatment comprised an immunotherapy (e.g., with an anti-PD-1 antibody). In other aspects, the prior cancer treatment comprised a chemotherapy. In some aspects, the tumor has reoccurred. In some aspects, the tumor is metastatic. In other aspects, the tumor is not metastatic.

In some aspects, methods disclosed herein effectively increases the duration of survival of a subject in need thereof (e.g., afflicted with a tumor). For example, in some aspects, duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more when compared to a reference individual (e.g., corresponding subject treated with IL-7 protein alone or with an immune checkpoint inhibitor alone). In other aspects, the methods disclosed herein increases duration of survival of the subject at a level higher than (about one month higher than, about two months higher than, about three months higher than, about four months higher than, about five months higher than, about six months higher than, about seven months higher than, about eight months higher than, about nine months higher than, about ten months higher than, about eleven months higher than, or about one year higher than) the duration of survival of a reference subject (e.g., corresponding subject treated with IL-7 protein alone or with an immune checkpoint inhibitor alone).

In some aspects, methods of the present disclosure effectively increase the duration of progression-free survival of a subject (e.g., cancer patient). For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year when compared to a reference subject (e.g., corresponding subject treated with IL-7 protein alone or with an immune checkpoint inhibitor alone).

In some aspects, methods disclosed herein effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to a reference subject (e.g., corresponding subject treated with IL-7 protein alone or with an immune checkpoint inhibitor alone).

In some aspects, the subject being treated in the method is a nonhuman animal such as a rat or a mouse. In some aspects, the subject being treated in the method is a human.

In some aspects, the unit dose (e.g., for human use) of an IL-7 protein disclosed herein can be in the range of 0.001 mg/kg to 10 mg/kg. In certain aspects, the unit dose of an IL-7 protein is in the range of 0.01 mg/kg to 2 mg/kg. In some aspects, the unit dose is in the range of 0.02 mg/kg to 1 mg/kg. The unit dose can vary depending on the subject diseases for treatment and the presence of adverse effects. The administration of an IL-7 protein can be performed by periodic bolus injections or external reservoirs (e.g., intravenous bags) or by continuous intravenous, subcutaneous, or intraperitoneal administration from the internal (e.g., biocorrosive implants). In certain aspects, an IL-7 protein disclosed herein is administered via intramuscular injection.

In some aspects, an IL-7 protein disclosed herein can be administered to a subject at a weight-based dose. In certain aspects, an IL-7 protein can be administered at a weight-based dose between about 20 μg/kg and about 600 μg/kg. In further aspects, an IL-7 protein of the present disclosure can be administered at a weight-based dose of about 20 μg/kg, about 60 μg/kg, about 120 μg/kg, about 240 μg/kg, about 360 μg/kg, about 480 μg/kg, or about 600 μg/kg.

In some aspects, an IL-7 protein disclosed herein can be administered to a subject at a dose greater than about 600 μg/kg. In certain aspects, an IL-7 protein is administered to a subject at a dose greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, an IL-7 protein of the present disclosure is administered at a dose of between 610 μg/kg and about 1,200 μg/kg, between 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, an IL-7 protein of the present disclosure is administered at a dose of between 610 μg/kg and about 1,200 μg/kg. In certain aspects, an IL-7 protein is administered at a dose of between 650 μg/kg and about 1,200 μg/kg. In some aspects, an IL-7 protein is administered at a dose of between about 700 μg/kg and about 1,200 μg/kg. In further aspects, an IL-7 protein is administered at a dose of between about 750 μg/kg and about 1,200 μg/kg. In certain aspects, an IL-7 protein is administered at a dose of between about 800 μg/kg and about 1,200 μg/kg. In some aspects, an IL-7 protein is administered at a dose of between about 850 μg/kg and about 1,200 μg/kg. In some aspects, an IL-7 protein is administered at a dose of between about 900 μg/kg and about 1,200 μg/kg. In further aspects, an IL-7 protein is administered at a dose of between about 950 μg/kg and about 1,200 μg/kg. In some aspects, an IL-7 protein disclosed herein is administered at a dose of between about 1,000 μg/kg and about 1,200 μg/kg. In some aspects, an IL-7 protein is administered at a dose of between about 1,050 μg/kg and about 1,200 μg/kg. In some aspects, an IL-7 protein is administered at a dose of between about 1,100 μg/kg and about 1,200 μg/kg. In some aspects, an IL-7 protein is administered at a dose of between about 1,200 μg/kg and about 2,000 μg/kg. In further aspects, an IL-7 protein is administered at a dose of between about 1,300 μg/kg and about 2,000 μg/kg. In some aspects, an IL-7 protein is administered at a dose of between about 1,500 μg/kg and about 2,000 μg/kg. In some aspects, an IL-7 protein is administered at a dose of between about 1,700 μg/kg and about 2,000 μg/kg. In certain aspects, an IL-7 protein is administered at a dose of between about 610 μg/kg and about 1,000 μg/kg. In some aspects, an IL-7 protein is administered at a dose of between about 650 μg/kg and about 1,000 μg/kg. In further aspects, an IL-7 protein is administered at a dose of between about 700 μg/kg and about 1,000 μg/kg. In yet further aspects, an IL-7 protein is administered at a dose of between about 750 μg/kg and about 1,000 μg/kg. In certain aspects, an IL-7 protein is administered at a dose of between about 800 μg/kg and about 1,000 μg/kg. In some aspects, an IL-7 protein is administered at a dose of between about 850 μg/kg and about 1,000 μg/kg. In some aspects, an IL-7 protein of the present disclosure is administered at a dose of between about 900 μg/kg and about 1,000 μg/kg. In some aspects, an IL-7 protein is administered at a dose of between about 950 μg/kg and about 1,000 μg/kg.

In some aspects, an IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, an IL-7 protein is administered at a dose of between about 700 μg/kg and about 900 μg/kg. In certain aspects, an IL-7 protein is administered at a dose of between about 750 μg/kg and about 950 μg/kg. In further aspects, an IL-7 protein is administered at a dose of between about 700 μg/kg and about 850 μg/kg. In some aspects, an IL-7 protein is administered at a dose of between about 750 μg/kg and about 850 μg/kg. In other aspects, an IL-7 protein is administered at a dose of between about 700 μg/kg and about 800 μg/kg. In some aspects, an IL-7 protein is administered at a dose of between about 800 μg/kg and about 900 μg/kg. In some aspects, an IL-7 protein is administered at a dose of between about 750 μg/kg and about 850 μg/kg. In certain aspects, an IL-7 protein is administered at a dose of between about 850 μg/kg and about 950 μg/kg.

In some aspects, an IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg. In some aspects, an IL-7 protein is administered at a dose of about 650 μg/kg. In other aspects, an IL-7 protein disclosed herein is administered at a dose of about 680 μg/kg. In some aspects, an IL-7 protein is administered at a dose of about 700 μg/kg. In some aspects, an IL-7 protein is administered at a dose of about 720 μg/kg. In certain aspects, an IL-7 protein is administered at a dose of about 740 μg/kg. In some aspects, an IL-7 protein is administered at a dose of about 750 μg/kg. In some aspects, an IL-7 protein is administered at a dose of about 760 μg/kg. In certain aspects, an IL-7 protein is administered at a dose of about 780 μg/kg. In some aspects, an IL-7 protein is administered at a dose of about 800 μg/kg. In further aspects, an IL-7 protein is administered at a dose of about 820 μg/kg. In certain aspects, an IL-7 protein is administered at a dose of about 840 μg/kg. In some aspects, an IL-7 protein is administered at a dose of about 850 μg/kg. In certain aspects, an IL-7 protein is administered at a dose of about 860 μg/kg. In some aspects, an IL-7 protein is administered at a dose of about 880 μg/kg. In some aspects, an IL-7 protein is administered at a dose of about 900 μg/kg. In further aspects, an IL-7 protein is administered at a dose of about 920 μg/kg. In some aspects, an IL-7 protein is administered at a dose of about 940 μg/kg. In further aspects, an IL-7 protein is administered at a dose of about 950 μg/kg. In some aspects, an IL-7 protein is administered at a dose of about 960 μg/kg. In certain aspects, an IL-7 protein is administered at a dose of about 980 μg/kg. In some aspects, an IL-7 protein is administered at a dose of about 1,000 μg/kg. In certain aspects, an IL-7 protein is administered at a dose of about 1,020 μg/kg. In further aspects, an IL-7 protein is administered at a dose of about 1,040 μg/kg. In some aspects, an IL-7 protein is administered at a dose of about 1,060 μg/kg. In other aspects, an IL-7 protein is administered at a dose of about 1,080 μg/kg. In some aspects, an IL-7 protein is administered at a dose of about 1,100 μg/kg. In certain aspects, an IL-7 protein is administered at a dose of about 1,120 μg/kg. In further aspects, an IL-7 protein is administered at a dose of about 1,140 μg/kg. In some aspects, an IL-7 protein is administered at a dose of about 1,160 μg/kg. In other aspects, an IL-7 protein is administered at a dose of about 1,180 μg/kg. In certain aspects, an IL-7 protein is administered at a dose of about 1200 μg/kg. In certain aspects, an IL-7 protein is administered at a dose of about 1,220 μg/kg. In further aspects, an IL-7 protein is administered at a dose of about 1,240 μg/kg. In some aspects, an IL-7 protein is administered at a dose of about 1,260 μg/kg. In other aspects, an IL-7 protein is administered at a dose of about 1,280 μg/kg. In some aspects, an IL-7 protein is administered at a dose of about 1,300 μg/kg. In certain aspects, an IL-7 protein is administered at a dose of about 1,320 μg/kg. In further aspects, an IL-7 protein is administered at a dose of about 1,340 μg/kg. In some aspects, an IL-7 protein is administered at a dose of about 1,360 μg/kg. In other aspects, an IL-7 protein is administered at a dose of about 1,380 μg/kg. In further aspects, an IL-7 protein is administered at a dose of about 1,400 μg/kg. In certain aspects, an IL-7 protein is administered at a dose of about 1,420 μg/kg. In further aspects, an IL-7 protein is administered at a dose of about 1,440 μg/kg. In some aspects, an IL-7 protein is administered at a dose of about 1,460 μg/kg. In other aspects, an IL-7 protein is administered at a dose of about 1,480 μg/kg. In certain aspects, an IL-7 protein is administered at a dose of about 1,500 μg/kg. In certain aspects, an IL-7 protein is administered at a dose of about 1,520 μg/kg. In further aspects, an IL-7 protein is administered at a dose of about 1,540 μg/kg. In some aspects, an IL-7 protein is administered at a dose of about 1,560 μg/kg. In other aspects, an IL-7 protein is administered at a dose of about 1,580 μg/kg. In some aspects, an IL-7 protein is administered at a dose of about 1,600 μg/kg. In certain aspects, an IL-7 protein is administered at a dose of about 1,620 μg/kg. In further aspects, an IL-7 protein is administered at a dose of about 1,640 μg/kg. In some aspects, an IL-7 protein is administered at a dose of about 1,660 μg/kg. In other aspects, an IL-7 protein is administered at a dose of about 1,680 μg/kg. In certain aspects, an IL-7 protein is administered at a dose of about 1,700 μg/kg. In certain aspects, an IL-7 protein is administered at a dose of about 1,720 μg/kg. In further aspects, an IL-7 protein is administered at a dose of about 1,740 μg/kg. In some aspects, an IL-7 protein is administered at a dose of about 1,760 μg/kg. In other aspects, an IL-7 protein is administered at a dose of about 1,780 μg/kg. In some aspects, an IL-7 protein is administered at a dose of about 1,800 μg/kg. In certain aspects, an IL-7 protein is administered at a dose of about 1,820 μg/kg. In further aspects, an IL-7 protein is administered at a dose of about 1,840 μg/kg. In some aspects, an IL-7 protein is administered at a dose of about 1,860 μg/kg. In other aspects, an IL-7 protein is administered at a dose of about 1,880 μg/kg. In some aspects, an IL-7 protein is administered at a dose of about 1,900 μg/kg. In certain aspects, an IL-7 protein is administered at a dose of about 1,920 μg/kg. In further aspects, an IL-7 protein is administered at a dose of about 1,940 μg/kg. In some aspects, an IL-7 protein is administered at a dose of about 1,960 μg/kg. In other aspects, an IL-7 protein is administered at a dose of about 1,980 μg/kg. In further aspects, an IL-7 protein is administered at a dose of about 2,000 μg/kg.

In some aspects, an IL-7 protein can be administered at a flat dose. In certain aspects, an IL-7 protein can be administered at a flat dose of about 0.25 mg to about 9 mg. In some aspects, an IL-7 protein can be administered at a flat dose of about 0.25 mg, about 1 mg, about 3 mg, about 6 mg, or about 9 mg.

In some aspects, an IL-7 protein disclosed herein is administered to a subject at multiple doses (i.e., repeated administrations). In certain embodiments, an IL-7 protein is administered to the subject at least two times, at least three times, at least four times, at least five times, at least six times, at least seven times, at least eight times, at least nine times, or at least ten times or more. In other embodiments, a subject receives a single administration of the IL-7 protein (e.g., prior to, concurrently, or after an administration of an immune checkpoint inhibitor).

In some aspects, an IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks. In certain aspects, an IL-7 protein is administered at a dosing frequency of about once every 10 days, about once every 20 days, about once every 30 days, about once every 40 days, about once every 50 days, about once every 60 days, about once every 70 days, about once every 80 days, about once every 90 days, or about once every 100 days. In some aspects, the IL-7 protein is administered once in three weeks. In some aspects, the IL-7 protein is administered once a week. In some aspects, the IL-7 protein is administered once in two weeks. In some aspects, the IL-7 protein is administered once in four weeks. In certain aspects, the IL-7 protein is administered once in six weeks. In further aspects, the IL-7 protein is administered once in eight weeks. In some aspects, the IL-7 protein is administered once in nine weeks. In certain aspects, the IL-7 protein is administered once in 12 weeks. In some aspects, the IL-7 protein is administered once every 10 days. In certain aspects, the IL-7 protein is administered once every 20 days. In other aspects, the IL-7 protein is administered once every 30 days. In some aspects, the IL-7 protein is administered once every 40 days. In certain aspects, the IL-7 protein is administered once every 50 days. In some aspects, the IL-7 protein is administered once every 60 days. In further aspects, the IL-7 protein is administered once every 70 days. In some aspects, the IL-7 protein is administered once every 80 days. In certain aspects, the IL-7 protein is administered once every 90 days. In some aspects, the IL-7 protein is administered once every 100 days.

In some aspects, the IL-7 protein is administered twice or more times in an amount of about 720 μg/kg at an interval of about 1 week, about 2 weeks, about 3 weeks, or about 4 weeks. In some aspects, the IL-7 protein is administered twice or more times in an amount of about 840 μg/kg at an interval of about 2 weeks, about 3 weeks, about 4 weeks, or about 5 weeks. In some aspects, the IL-7 protein is administered twice or more times in an amount of about 960 μg/kg at an interval of about 2 weeks, about 3 weeks, about 4 weeks, about 5 weeks, or about 6 weeks. In some aspects, the IL-7 protein is administered twice or more times in an amount of about 1200 μg/kg at an interval of about 3 weeks, about 4 weeks, about 5 weeks, about 6 weeks, about 7 weeks, or about 8 weeks. In some aspects, the IL-7 protein is administered twice or more times in an amount of about 1440 μg/kg at an interval of about 3 weeks, about 4 weeks, about 5 weeks, about 6 weeks, about 7 weeks, about 8 weeks, about 2 months, about 8 weeks, about 10 weeks, about 12 weeks, or about 3 months.

In some aspects, the IL-7 protein is administered at a dose of 60 μg/kg with a dosing frequency of once a week. In some aspects, the IL-7 protein is administered at a dose of 120 μg/kg with a dosing frequency of once a week. In some aspects, the IL-7 protein is administered at a dose of 240 μg/kg with a dosing frequency of once a week. In some aspects, the IL-7 protein is administered at a dose of 480 μg/kg with a dosing frequency of once a week. In some aspects, the IL-7 protein is administered at a dose of 720 μg/kg with a dosing frequency of once a week. In some aspects, the IL-7 protein is administered at a dose of 960 μg/kg with a dosing frequency of once a week. In some aspects, the IL-7 protein is administered at a dose of 1,200 μg/kg with a dosing frequency of once a week. In some aspects, the IL-7 protein is administered at a dose of 1,300 μg/kg with a dosing frequency of once a week. In some aspects, the IL-7 protein is administered at a dose of 1,400 μg/kg with a dosing frequency of once a week. In other aspects, the IL-7 protein is administered at a dose of 1,420 μg/kg with a dosing frequency of once a week. In certain aspects, the IL-7 protein is administered at a dose of 1,440 μg/kg with a dosing frequency of once a week. In further aspects, the IL-7 protein is administered at a dose of 1,460 μg/kg with a dosing frequency of once a week. In certain aspects, the IL-7 protein is administered at a dose of 1,480 μg/kg with a dosing frequency of once a week. In some aspects, the IL-7 protein is administered at a dose of 1,500 μg/kg with a dosing frequency of once a week. In further aspects, the IL-7 protein is administered at a dose of 1,600 μg/kg with a dosing frequency of once a week. In some aspects, the IL-7 protein is administered at a dose of 1,700 μg/kg with a dosing frequency of once a week. In some aspects, the IL-7 protein is administered at a dose of 2,000 μg/kg with a dosing frequency of once a week.

In some aspects, the IL-7 protein is administered at a dose of 60 μg/kg with a dosing frequency of once in two weeks. In some aspects, the IL-7 protein is administered at a dose of 120 μg/kg with a dosing frequency of once in two weeks. In some aspects, the IL-7 protein is administered at a dose of 240 μg/kg with a dosing frequency of once in two weeks. In some aspects, the IL-7 protein is administered at a dose of 480 μg/kg with a dosing frequency of once in two weeks. In some aspects, the IL-7 protein is administered at a dose of 720 μg/kg with a dosing frequency of once in two weeks. In some aspects, the IL-7 protein is administered at a dose of 960 μg/kg with a dosing frequency of once in two weeks. In some aspects, the IL-7 protein is administered at a dose of 1,200 μg/kg with a dosing frequency of once in two weeks. In some aspects, the IL-7 protein is administered at a dose of 1,300 μg/kg with a dosing frequency of once in two weeks. In some aspects, the IL-7 protein is administered at a dose of 1,400 μg/kg with a dosing frequency of once in two weeks. In other aspects, the IL-7 protein is administered at a dose of 1,420 μg/kg with a dosing frequency of once in two weeks. In certain aspects, the IL-7 protein is administered at a dose of 1,440 μg/kg with a dosing frequency of once in two weeks. In further aspects, the IL-7 protein is administered at a dose of 1,460 μg/kg with a dosing frequency of once in two weeks. In certain aspects, the IL-7 protein is administered at a dose of 1,480 μg/kg with a dosing frequency of once in two weeks. In some aspects, the IL-7 protein is administered at a dose of 1,500 μg/kg with a dosing frequency of once in two weeks. In further aspects, the IL-7 protein is administered at a dose of 1,600 μg/kg with a dosing frequency of once in two weeks. In some aspects, the IL-7 protein is administered at a dose of 1,700 μg/kg with a dosing frequency of once in two weeks. In some aspects, the IL-7 protein is administered at a dose of 2,000 μg/kg with a dosing frequency of once in two weeks.

In some aspects, the IL-7 protein is administered at a dose of 60 μg/kg with a dosing frequency of once in three weeks. In some aspects, the IL-7 protein is administered at a dose of 120 μg/kg with a dosing frequency of once in three weeks. In some aspects, the IL-7 protein is administered at a dose of 240 μg/kg with a dosing frequency of once in three weeks. In some aspects, the IL-7 protein is administered at a dose of 480 μg/kg with a dosing frequency of once in three weeks. In some aspects, the IL-7 protein is administered at a dose of 720 μg/kg with a dosing frequency of once in three weeks. In some aspects, the IL-7 protein is administered at a dose of 960 μg/kg with a dosing frequency of once in three weeks. In some aspects, the IL-7 protein is administered at a dose of 1,200 μg/kg with a dosing frequency of once in three weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In other aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in three weeks. In further aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In further aspects, the IL-7 protein is administered at a dose of 1,600 μg/kg with a dosing frequency of once in three weeks. In some aspects, the IL-7 protein is administered at a dose of 1,700 μg/kg with a dosing frequency of once in three weeks. In some aspects, the IL-7 protein is administered at a dose of 2,000 μg/kg with a dosing frequency of once in three weeks.

In some aspects, the IL-7 protein is administered at a dose of 60 μg/kg with a dosing frequency of once in four weeks. In some aspects, the IL-7 protein is administered at a dose of 120 μg/kg with a dosing frequency of once in four weeks. In some aspects, the IL-7 protein is administered at a dose of 240 μg/kg with a dosing frequency of once in four weeks. In some aspects, the IL-7 protein is administered at a dose of 480 μg/kg with a dosing frequency of once in four weeks. In some aspects, the IL-7 protein is administered at a dose of 720 μg/kg with a dosing frequency of once in four weeks. In some aspects, the IL-7 protein is administered at a dose of 960 μg/kg with a dosing frequency of once in four weeks. In some aspects, the IL-7 protein is administered at a dose of 1,200 μg/kg with a dosing frequency of once in four weeks. In some aspects, the IL-7 protein is administered at a dose of 1,300 μg/kg with a dosing frequency of once in four weeks. In some aspects, the IL-7 protein is administered at a dose of 1,400 μg/kg with a dosing frequency of once in four weeks. In other aspects, the IL-7 protein is administered at a dose of 1,420 μg/kg with a dosing frequency of once in four weeks. In certain aspects, the IL-7 protein is administered at a dose of 1,440 μg/kg with a dosing frequency of once in four weeks. In further aspects, the IL-7 protein is administered at a dose of 1,460 μg/kg with a dosing frequency of once in four weeks. In certain aspects, the IL-7 protein is administered at a dose of 1,480 μg/kg with a dosing frequency of once in four weeks. In some aspects, the IL-7 protein is administered at a dose of 1,500 μg/kg with a dosing frequency of once in four weeks. In further aspects, the IL-7 protein is administered at a dose of 1,600 μg/kg with a dosing frequency of once in four weeks. In some aspects, the IL-7 protein is administered at a dose of 1,700 μg/kg with a dosing frequency of once in four weeks. In some aspects, the IL-7 protein is administered at a dose of 2,000 μg/kg with a dosing frequency of once in four weeks.

In some aspects, the IL-7 protein is administered at a dose of 60 μg/kg with a dosing frequency of once in five weeks. In some aspects, the IL-7 protein is administered at a dose of 120 μg/kg with a dosing frequency of once in five weeks. In some aspects, the IL-7 protein is administered at a dose of 240 μg/kg with a dosing frequency of once in five weeks. In some aspects, the IL-7 protein is administered at a dose of 480 μg/kg with a dosing frequency of once in five weeks. In some aspects, the IL-7 protein is administered at a dose of 720 μg/kg with a dosing frequency of once in five weeks. In some aspects, the IL-7 protein is administered at a dose of 960 μg/kg with a dosing frequency of once in five weeks. In some aspects, the IL-7 protein is administered at a dose of 1,200 μg/kg with a dosing frequency of once in five weeks. In some aspects, the IL-7 protein is administered at a dose of 1,300 μg/kg with a dosing frequency of once in five weeks. In some aspects, the IL-7 protein is administered at a dose of 1,400 μg/kg with a dosing frequency of once in five weeks. In other aspects, the IL-7 protein is administered at a dose of 1,420 μg/kg with a dosing frequency of once in five weeks. In certain aspects, the IL-7 protein is administered at a dose of 1,440 μg/kg with a dosing frequency of once in five weeks. In further aspects, the IL-7 protein is administered at a dose of 1,460 μg/kg with a dosing frequency of once in five weeks. In certain aspects, the IL-7 protein is administered at a dose of 1,480 μg/kg with a dosing frequency of once in five weeks. In some aspects, the IL-7 protein is administered at a dose of 1,500 μg/kg with a dosing frequency of once in five weeks. In further aspects, the IL-7 protein is administered at a dose of 1,600 μg/kg with a dosing frequency of once in five weeks. In some aspects, the IL-7 protein is administered at a dose of 1,700 μg/kg with a dosing frequency of once in five weeks. In some aspects, the IL-7 protein is administered at a dose of 2,000 μg/kg with a dosing frequency of once in five weeks.

In some aspects, the IL-7 protein is administered at a dose of 60 μg/kg with a dosing frequency of once in six weeks. In some aspects, the IL-7 protein is administered at a dose of 120 μg/kg with a dosing frequency of once in six weeks. In some aspects, the IL-7 protein is administered at a dose of 240 μg/kg with a dosing frequency of once in six weeks. In some aspects, the IL-7 protein is administered at a dose of 480 μg/kg with a dosing frequency of once in six weeks. In some aspects, the IL-7 protein is administered at a dose of 720 μg/kg with a dosing frequency of once in six weeks. In some aspects, the IL-7 protein is administered at a dose of 960 μg/kg with a dosing frequency of once in six weeks. In some aspects, the IL-7 protein is administered at a dose of 1,200 μg/kg with a dosing frequency of once in six weeks. In some aspects, the IL-7 protein is administered at a dose of 1,300 μg/kg with a dosing frequency of once in six weeks. In some aspects, the IL-7 protein is administered at a dose of 1,400 μg/kg with a dosing frequency of once in six weeks. In other aspects, the IL-7 protein is administered at a dose of 1,420 μg/kg with a dosing frequency of once in six weeks. In certain aspects, the IL-7 protein is administered at a dose of 1,440 μg/kg with a dosing frequency of once in six weeks. In further aspects, the IL-7 protein is administered at a dose of 1,460 μg/kg with a dosing frequency of once in six weeks. In certain aspects, the IL-7 protein is administered at a dose of 1,480 μg/kg with a dosing frequency of once in six weeks. In some aspects, the IL-7 protein is administered at a dose of 1,500 μg/kg with a dosing frequency of once in six weeks. In further aspects, the IL-7 protein is administered at a dose of 1,600 μg/kg with a dosing frequency of once in six weeks. In some aspects, the IL-7 protein is administered at a dose of 1,700 μg/kg with a dosing frequency of once in six weeks. In some aspects, the IL-7 protein is administered at a dose of 2,000 μg/kg with a dosing frequency of once in six weeks.

In some aspects, the IL-7 protein is administered at a dose of 60 μg/kg with a dosing frequency of once in seven weeks. In some aspects, the IL-7 protein is administered at a dose of 120 μg/kg with a dosing frequency of once in seven weeks. In some aspects, the IL-7 protein is administered at a dose of 240 μg/kg with a dosing frequency of once in seven weeks. In some aspects, the IL-7 protein is administered at a dose of 480 μg/kg with a dosing frequency of once in seven weeks. In some aspects, the IL-7 protein is administered at a dose of 720 μg/kg with a dosing frequency of once in seven weeks. In some aspects, the IL-7 protein is administered at a dose of 960 μg/kg with a dosing frequency of once in seven weeks. In some aspects, the IL-7 protein is administered at a dose of 1,200 μg/kg with a dosing frequency of once in seven weeks. In some aspects, the IL-7 protein is administered at a dose of 1,300 μg/kg with a dosing frequency of once in seven weeks. In some aspects, the IL-7 protein is administered at a dose of 1,400 μg/kg with a dosing frequency of once in seven weeks. In other aspects, the IL-7 protein is administered at a dose of 1,420 μg/kg with a dosing frequency of once in seven weeks. In certain aspects, the IL-7 protein is administered at a dose of 1,440 μg/kg with a dosing frequency of once in seven weeks. In further aspects, the IL-7 protein is administered at a dose of 1,460 μg/kg with a dosing frequency of once in seven weeks. In certain aspects, the IL-7 protein is administered at a dose of 1,480 μg/kg with a dosing frequency of once in seven weeks. In some aspects, the IL-7 protein is administered at a dose of 1,500 μg/kg with a dosing frequency of once in seven weeks. In further aspects, the IL-7 protein is administered at a dose of 1,600 μg/kg with a dosing frequency of once in seven weeks. In some aspects, the IL-7 protein is administered at a dose of 1,700 μg/kg with a dosing frequency of once in seven weeks. In some aspects, the IL-7 protein is administered at a dose of 2,000 μg/kg with a dosing frequency of once in seven weeks.

In some aspects, the IL-7 protein is administered at a dose of 60 μg/kg with a dosing frequency of once in eight weeks. In some aspects, the IL-7 protein is administered at a dose of 120 μg/kg with a dosing frequency of once in eight weeks. In some aspects, the IL-7 protein is administered at a dose of 240 μg/kg with a dosing frequency of once in eight weeks. In some aspects, the IL-7 protein is administered at a dose of 480 μg/kg with a dosing frequency of once in eight weeks. In some aspects, the IL-7 protein is administered at a dose of 720 μg/kg with a dosing frequency of once in eight weeks. In some aspects, the IL-7 protein is administered at a dose of 960 μg/kg with a dosing frequency of once in eight weeks. In some aspects, the IL-7 protein is administered at a dose of 1,200 μg/kg with a dosing frequency of once in eight weeks. In some aspects, the IL-7 protein is administered at a dose of 1,300 μg/kg with a dosing frequency of once in eight weeks. In some aspects, the IL-7 protein is administered at a dose of 1,400 μg/kg with a dosing frequency of once in eight weeks. In other aspects, the IL-7 protein is administered at a dose of 1,420 μg/kg with a dosing frequency of once in eight weeks. In certain aspects, the IL-7 protein is administered at a dose of 1,440 μg/kg with a dosing frequency of once in eight weeks. In further aspects, the IL-7 protein is administered at a dose of 1,460 μg/kg with a dosing frequency of once in eight weeks. In certain aspects, the IL-7 protein is administered at a dose of 1,480 μg/kg with a dosing frequency of once in eight weeks. In some aspects, the IL-7 protein is administered at a dose of 1,500 μg/kg with a dosing frequency of once in eight weeks. In further aspects, the IL-7 protein is administered at a dose of 1,600 μg/kg with a dosing frequency of once in eight weeks. In some aspects, the IL-7 protein is administered at a dose of 1,700 μg/kg with a dosing frequency of once in eight weeks. In some aspects, the IL-7 protein is administered at a dose of 2,000 μg/kg with a dosing frequency of once in eight weeks.

In some aspects, the IL-7 protein is administered at a dose of 60 μg/kg with a dosing frequency of once in nine weeks. In some aspects, the IL-7 protein is administered at a dose of 120 μg/kg with a dosing frequency of once in nine weeks. In some aspects, the IL-7 protein is administered at a dose of 240 μg/kg with a dosing frequency of once in nine weeks. In some aspects, the IL-7 protein is administered at a dose of 480 μg/kg with a dosing frequency of once in nine weeks. In some aspects, the IL-7 protein is administered at a dose of 720 μg/kg with a dosing frequency of once in nine weeks. In some aspects, the IL-7 protein is administered at a dose of 960 μg/kg with a dosing frequency of once in nine weeks. In some aspects, the IL-7 protein is administered at a dose of 1,200 μg/kg with a dosing frequency of once in nine weeks. In some aspects, the IL-7 protein is administered at a dose of 1,300 μg/kg with a dosing frequency of once in nine weeks. In some aspects, the IL-7 protein is administered at a dose of 1,400 μg/kg with a dosing frequency of once in nine weeks. In other aspects, the IL-7 protein is administered at a dose of 1,420 μg/kg with a dosing frequency of once in nine weeks. In certain aspects, the IL-7 protein is administered at a dose of 1,440 μg/kg with a dosing frequency of once in nine weeks. In further aspects, the IL-7 protein is administered at a dose of 1,460 μg/kg with a dosing frequency of once in nine weeks. In certain aspects, the IL-7 protein is administered at a dose of 1,480 μg/kg with a dosing frequency of once in nine weeks. In some aspects, the IL-7 protein is administered at a dose of 1,500 μg/kg with a dosing frequency of once in nine weeks. In further aspects, the IL-7 protein is administered at a dose of 1,600 μg/kg with a dosing frequency of once in nine weeks. In some aspects, the IL-7 protein is administered at a dose of 1,700 μg/kg with a dosing frequency of once in nine weeks. In some aspects, the IL-7 protein is administered at a dose of 2,000 μg/kg with a dosing frequency of once in nine weeks.

In some aspects, the IL-7 protein is administered at a dose of 60 μg/kg with a dosing frequency of once in 10 weeks. In some aspects, the IL-7 protein is administered at a dose of 120 μg/kg with a dosing frequency of once in 10 weeks. In some aspects, the IL-7 protein is administered at a dose of 240 μg/kg with a dosing frequency of once in 10 weeks. In some aspects, the IL-7 protein is administered at a dose of 480 μg/kg with a dosing frequency of once in 10 weeks. In some aspects, the IL-7 protein is administered at a dose of 720 μg/kg with a dosing frequency of once in 10 weeks. In some aspects, the IL-7 protein is administered at a dose of 960 μg/kg with a dosing frequency of once in 10 weeks. In some aspects, the IL-7 protein is administered at a dose of 1,200 μg/kg with a dosing frequency of once in 10 weeks. In some aspects, the IL-7 protein is administered at a dose of 1,300 μg/kg with a dosing frequency of once in 10 weeks. In some aspects, the IL-7 protein is administered at a dose of 1,400 μg/kg with a dosing frequency of once in 10 weeks. In other aspects, the IL-7 protein is administered at a dose of 1,420 μg/kg with a dosing frequency of once in 10 weeks. In certain aspects, the IL-7 protein is administered at a dose of 1,440 μg/kg with a dosing frequency of once in 10 weeks. In further aspects, the IL-7 protein is administered at a dose of 1,460 μg/kg with a dosing frequency of once in 10 weeks. In certain aspects, the IL-7 protein is administered at a dose of 1,480 μg/kg with a dosing frequency of once in 10 weeks. In some aspects, the IL-7 protein is administered at a dose of 1,500 μg/kg with a dosing frequency of once in 10 weeks. In further aspects, the IL-7 protein is administered at a dose of 1,600 μg/kg with a dosing frequency of once in 10 weeks. In some aspects, the IL-7 protein is administered at a dose of 1,700 μg/kg with a dosing frequency of once in 10 weeks. In some aspects, the IL-7 protein is administered at a dose of 2,000 μg/kg with a dosing frequency of once in 10 weeks.

In some aspects, the IL-7 protein is administered at a dose of 60 μg/kg with a dosing frequency of once in 11 weeks. In some aspects, the IL-7 protein is administered at a dose of 120 μg/kg with a dosing frequency of once in 11 weeks. In some aspects, the IL-7 protein is administered at a dose of 240 μg/kg with a dosing frequency of once in 11 weeks. In some aspects, the IL-7 protein is administered at a dose of 480 μg/kg with a dosing frequency of once in 11 weeks. In some aspects, the IL-7 protein is administered at a dose of 720 μg/kg with a dosing frequency of once in 11 weeks. In some aspects, the IL-7 protein is administered at a dose of 960 μg/kg with a dosing frequency of once in 11 weeks. In some aspects, the IL-7 protein is administered at a dose of 1,200 μg/kg with a dosing frequency of once in 11 weeks. In some aspects, the IL-7 protein is administered at a dose of 1,300 μg/kg with a dosing frequency of once in 11 weeks. In some aspects, the IL-7 protein is administered at a dose of 1,400 μg/kg with a dosing frequency of once in 11 weeks. In other aspects, the IL-7 protein is administered at a dose of 1,420 μg/kg with a dosing frequency of once in 11 weeks. In certain aspects, the IL-7 protein is administered at a dose of 1,440 μg/kg with a dosing frequency of once in 11 weeks. In further aspects, the IL-7 protein is administered at a dose of 1,460 μg/kg with a dosing frequency of once in 11 weeks. In certain aspects, the IL-7 protein is administered at a dose of 1,480 μg/kg with a dosing frequency of once in 11 weeks. In some aspects, the IL-7 protein is administered at a dose of 1,500 μg/kg with a dosing frequency of once in 11 weeks. In further aspects, the IL-7 protein is administered at a dose of 1,600 μg/kg with a dosing frequency of once in 11 weeks. In some aspects, the IL-7 protein is administered at a dose of 1,700 μg/kg with a dosing frequency of once in 11 weeks. In some aspects, the IL-7 protein is administered at a dose of 2,000 μg/kg with a dosing frequency of once in 11 weeks.

In some aspects, the IL-7 protein is administered at a dose of 60 μg/kg with a dosing frequency of once in 12 weeks. In some aspects, the IL-7 protein is administered at a dose of 120 μg/kg with a dosing frequency of once in 12 weeks. In some aspects, the IL-7 protein is administered at a dose of 240 μg/kg with a dosing frequency of once in 12 weeks. In some aspects, the IL-7 protein is administered at a dose of 480 μg/kg with a dosing frequency of once in 12 weeks. In some aspects, the IL-7 protein is administered at a dose of 720 μg/kg with a dosing frequency of once in 12 weeks. In some aspects, the IL-7 protein is administered at a dose of 960 μg/kg with a dosing frequency of once in 12 weeks. In some aspects, the IL-7 protein is administered at a dose of 1,200 μg/kg with a dosing frequency of once in 12 weeks. In some aspects, the IL-7 protein is administered at a dose of 1,300 μg/kg with a dosing frequency of once in 12 weeks. In some aspects, the IL-7 protein is administered at a dose of 1,400 μg/kg with a dosing frequency of once in 12 weeks. In other aspects, the IL-7 protein is administered at a dose of 1,420 μg/kg with a dosing frequency of once in 12 weeks. In certain aspects, the IL-7 protein is administered at a dose of 1,440 μg/kg with a dosing frequency of once in 12 weeks. In further aspects, the IL-7 protein is administered at a dose of 1,460 μg/kg with a dosing frequency of once in 12 weeks. In certain aspects, the IL-7 protein is administered at a dose of 1,480 μg/kg with a dosing frequency of once in 12 weeks. In some aspects, the IL-7 protein is administered at a dose of 1,500 μg/kg with a dosing frequency of once in 12 weeks. In further aspects, the IL-7 protein is administered at a dose of 1,600 μg/kg with a dosing frequency of once in 12 weeks. In some aspects, the IL-7 protein is administered at a dose of 1,700 μg/kg with a dosing frequency of once in 12 weeks. In some aspects, the IL-7 protein is administered at a dose of 2,000 μg/kg with a dosing frequency of once in 12 weeks.

In some aspects, the IL-7 protein is administered at a dose of 60 μg/kg with a dosing frequency of once every 10 days. In some aspects, the IL-7 protein is administered at a dose of 120 μg/kg with a dosing frequency of once every 10 days. In some aspects, the IL-7 protein is administered at a dose of 240 μg/kg with a dosing frequency of once every 10 days. In some aspects, the IL-7 protein is administered at a dose of 480 μg/kg with a dosing frequency of once every 10 days. In some aspects, the IL-7 protein is administered at a dose of 720 μg/kg with a dosing frequency of once every 10 days. In some aspects, the IL-7 protein is administered at a dose of 960 μg/kg with a dosing frequency of once every 10 days. In some aspects, the IL-7 protein is administered at a dose of 1,200 μg/kg with a dosing frequency of once every 10 days. In some aspects, the IL-7 protein is administered at a dose of 1,300 μg/kg with a dosing frequency of once every 10 days. In some aspects, the IL-7 protein is administered at a dose of 1,400 μg/kg with a dosing frequency of once every 10 days. In other aspects, the IL-7 protein is administered at a dose of 1,420 μg/kg with a dosing frequency of once every 10 days. In certain aspects, the IL-7 protein is administered at a dose of 1,440 μg/kg with a dosing frequency of once every 10 days. In further aspects, the IL-7 protein is administered at a dose of 1,460 μg/kg with a dosing frequency of once every 10 days. In certain aspects, the IL-7 protein is administered at a dose of 1,480 μg/kg with a dosing frequency of once every 10 days. In some aspects, the IL-7 protein is administered at a dose of 1,500 μg/kg with a dosing frequency of once every 10 days. In further aspects, the IL-7 protein is administered at a dose of 1,600 μg/kg with a dosing frequency of once every 10 days. In some aspects, the IL-7 protein is administered at a dose of 1,700 μg/kg with a dosing frequency of once every 10 days. In some aspects, the IL-7 protein is administered at a dose of 2,000 μg/kg with a dosing frequency of once every 10 days.

In some aspects, the IL-7 protein is administered at a dose of 60 μg/kg with a dosing frequency of once every 20 days. In some aspects, the IL-7 protein is administered at a dose of 120 μg/kg with a dosing frequency of once every 20 days. In some aspects, the IL-7 protein is administered at a dose of 240 μg/kg with a dosing frequency of once every 20 days. In some aspects, the IL-7 protein is administered at a dose of 480 μg/kg with a dosing frequency of once every 20 days. In some aspects, the IL-7 protein is administered at a dose of 720 μg/kg with a dosing frequency of once every 20 days. In some aspects, the IL-7 protein is administered at a dose of 960 μg/kg with a dosing frequency of once every 20 days. In some aspects, the IL-7 protein is administered at a dose of 1,200 μg/kg with a dosing frequency of once every 20 days. In some aspects, the IL-7 protein is administered at a dose of 1,300 μg/kg with a dosing frequency of once every 20 days. In some aspects, the IL-7 protein is administered at a dose of 1,400 μg/kg with a dosing frequency of once every 20 days. In other aspects, the IL-7 protein is administered at a dose of 1,420 μg/kg with a dosing frequency of once every 20 days. In certain aspects, the IL-7 protein is administered at a dose of 1,440 μg/kg with a dosing frequency of once every 20 days. In further aspects, the IL-7 protein is administered at a dose of 1,460 μg/kg with a dosing frequency of once every 20 days. In certain aspects, the IL-7 protein is administered at a dose of 1,480 μg/kg with a dosing frequency of once every 20 days. In some aspects, the IL-7 protein is administered at a dose of 1,500 μg/kg with a dosing frequency of once every 20 days. In further aspects, the IL-7 protein is administered at a dose of 1,600 μg/kg with a dosing frequency of once every 20 days. In some aspects, the IL-7 protein is administered at a dose of 1,700 μg/kg with a dosing frequency of once every 20 days. In some aspects, the IL-7 protein is administered at a dose of 2,000 μg/kg with a dosing frequency of once every 20 days.

In some aspects, the IL-7 protein is administered at a dose of 60 μg/kg with a dosing frequency of once every 30 days. In some aspects, the IL-7 protein is administered at a dose of 120 μg/kg with a dosing frequency of once every 30 days. In some aspects, the IL-7 protein is administered at a dose of 240 μg/kg with a dosing frequency of once every 30 days. In some aspects, the IL-7 protein is administered at a dose of 480 μg/kg with a dosing frequency of once every 30 days. In some aspects, the IL-7 protein is administered at a dose of 720 μg/kg with a dosing frequency of once every 30 days. In some aspects, the IL-7 protein is administered at a dose of 960 μg/kg with a dosing frequency of once every 30 days. In some aspects, the IL-7 protein is administered at a dose of 1,200 μg/kg with a dosing frequency of once every 30 days. In some aspects, the IL-7 protein is administered at a dose of 1,300 μg/kg with a dosing frequency of once every 30 days. In some aspects, the IL-7 protein is administered at a dose of 1,400 μg/kg with a dosing frequency of once every 30 days. In other aspects, the IL-7 protein is administered at a dose of 1,420 μg/kg with a dosing frequency of once every 30 days. In certain aspects, the IL-7 protein is administered at a dose of 1,440 μg/kg with a dosing frequency of once every 30 days. In further aspects, the IL-7 protein is administered at a dose of 1,460 μg/kg with a dosing frequency of once every 30 days. In certain aspects, the IL-7 protein is administered at a dose of 1,480 μg/kg with a dosing frequency of once every 30 days. In some aspects, the IL-7 protein is administered at a dose of 1,500 μg/kg with a dosing frequency of once every 30 days. In further aspects, the IL-7 protein is administered at a dose of 1,600 μg/kg with a dosing frequency of once every 30 days. In some aspects, the IL-7 protein is administered at a dose of 1,700 μg/kg with a dosing frequency of once every 30 days. In some aspects, the IL-7 protein is administered at a dose of 2,000 μg/kg with a dosing frequency of once every 30 days.

In some aspects, the IL-7 protein is administered at a dose of 60 μg/kg with a dosing frequency of once every 40 days. In some aspects, the IL-7 protein is administered at a dose of 120 μg/kg with a dosing frequency of once every 40 days. In some aspects, the IL-7 protein is administered at a dose of 240 μg/kg with a dosing frequency of once every 40 days. In some aspects, the IL-7 protein is administered at a dose of 480 μg/kg with a dosing frequency of once every 40 days. In some aspects, the IL-7 protein is administered at a dose of 720 μg/kg with a dosing frequency of once every 40 days. In some aspects, the IL-7 protein is administered at a dose of 960 μg/kg with a dosing frequency of once every 40 days. In some aspects, the IL-7 protein is administered at a dose of 1,200 μg/kg with a dosing frequency of once every 40 days. In some aspects, the IL-7 protein is administered at a dose of 1,300 μg/kg with a dosing frequency of once every 40 days. In some aspects, the IL-7 protein is administered at a dose of 1,400 μg/kg with a dosing frequency of once every 40 days. In other aspects, the IL-7 protein is administered at a dose of 1,420 μg/kg with a dosing frequency of once every 40 days. In certain aspects, the IL-7 protein is administered at a dose of 1,440 μg/kg with a dosing frequency of once every 40 days. In further aspects, the IL-7 protein is administered at a dose of 1,460 μg/kg with a dosing frequency of once every 40 days. In certain aspects, the IL-7 protein is administered at a dose of 1,480 μg/kg with a dosing frequency of once every 40 days. In some aspects, the IL-7 protein is administered at a dose of 1,500 μg/kg with a dosing frequency of once every 40 days. In further aspects, the IL-7 protein is administered at a dose of 1,600 μg/kg with a dosing frequency of once every 40 days. In some aspects, the IL-7 protein is administered at a dose of 1,700 μg/kg with a dosing frequency of once every 40 days. In some aspects, the IL-7 protein is administered at a dose of 2,000 μg/kg with a dosing frequency of once every 40 days.

In some aspects, the IL-7 protein is administered at a dose of 60 μg/kg with a dosing frequency of once every 50 days. In some aspects, the IL-7 protein is administered at a dose of 120 μg/kg with a dosing frequency of once every 50 days. In some aspects, the IL-7 protein is administered at a dose of 240 μg/kg with a dosing frequency of once every 50 days. In some aspects, the IL-7 protein is administered at a dose of 480 μg/kg with a dosing frequency of once every 50 days. In some aspects, the IL-7 protein is administered at a dose of 720 μg/kg with a dosing frequency of once every 50 days. In some aspects, the IL-7 protein is administered at a dose of 960 μg/kg with a dosing frequency of once every 50 days. In some aspects, the IL-7 protein is administered at a dose of 1,200 μg/kg with a dosing frequency of once every 50 days. In some aspects, the IL-7 protein is administered at a dose of 1,300 μg/kg with a dosing frequency of once every 50 days. In some aspects, the IL-7 protein is administered at a dose of 1,400 μg/kg with a dosing frequency of once every 50 days. In other aspects, the IL-7 protein is administered at a dose of 1,420 μg/kg with a dosing frequency of once every 50 days. In certain aspects, the IL-7 protein is administered at a dose of 1,440 μg/kg with a dosing frequency of once every 50 days. In further aspects, the IL-7 protein is administered at a dose of 1,460 μg/kg with a dosing frequency of once every 50 days. In certain aspects, the IL-7 protein is administered at a dose of 1,480 μg/kg with a dosing frequency of once every 50 days. In some aspects, the IL-7 protein is administered at a dose of 1,500 μg/kg with a dosing frequency of once every 50 days. In further aspects, the IL-7 protein is administered at a dose of 1,600 μg/kg with a dosing frequency of once every 50 days. In some aspects, the IL-7 protein is administered at a dose of 1,700 μg/kg with a dosing frequency of once every 50 days. In some aspects, the IL-7 protein is administered at a dose of 2,000 μg/kg with a dosing frequency of once every 50 days.

In some aspects, the IL-7 protein is administered at a dose of 60 μg/kg with a dosing frequency of once every 60 days. In some aspects, the IL-7 protein is administered at a dose of 120 μg/kg with a dosing frequency of once every 60 days. In some aspects, the IL-7 protein is administered at a dose of 240 μg/kg with a dosing frequency of once every 60 days. In some aspects, the IL-7 protein is administered at a dose of 480 μg/kg with a dosing frequency of once every 60 days. In some aspects, the IL-7 protein is administered at a dose of 720 μg/kg with a dosing frequency of once every 60 days. In some aspects, the IL-7 protein is administered at a dose of 960 μg/kg with a dosing frequency of once every 60 days. In some aspects, the IL-7 protein is administered at a dose of 1,200 μg/kg with a dosing frequency of once every 60 days. In some aspects, the IL-7 protein is administered at a dose of 1,300 μg/kg with a dosing frequency of once every 60 days. In some aspects, the IL-7 protein is administered at a dose of 1,400 μg/kg with a dosing frequency of once every 60 days. In other aspects, the IL-7 protein is administered at a dose of 1,420 μg/kg with a dosing frequency of once every 60 days. In certain aspects, the IL-7 protein is administered at a dose of 1,440 μg/kg with a dosing frequency of once every 60 days. In further aspects, the IL-7 protein is administered at a dose of 1,460 μg/kg with a dosing frequency of once every 60 days. In certain aspects, the IL-7 protein is administered at a dose of 1,480 μg/kg with a dosing frequency of once every 60 days. In some aspects, the IL-7 protein is administered at a dose of 1,500 μg/kg with a dosing frequency of once every 60 days. In further aspects, the IL-7 protein is administered at a dose of 1,600 μg/kg with a dosing frequency of once every 60 days. In some aspects, the IL-7 protein is administered at a dose of 1,700 μg/kg with a dosing frequency of once every 60 days. In some aspects, the IL-7 protein is administered at a dose of 2,000 μg/kg with a dosing frequency of once every 60 days.

In some aspects, the IL-7 protein is administered at a dose of 60 μg/kg with a dosing frequency of once every 70 days. In some aspects, the IL-7 protein is administered at a dose of 120 μg/kg with a dosing frequency of once every 70 days. In some aspects, the IL-7 protein is administered at a dose of 240 μg/kg with a dosing frequency of once every 70 days. In some aspects, the IL-7 protein is administered at a dose of 480 μg/kg with a dosing frequency of once every 70 days. In some aspects, the IL-7 protein is administered at a dose of 720 μg/kg with a dosing frequency of once every 70 days. In some aspects, the IL-7 protein is administered at a dose of 960 μg/kg with a dosing frequency of once every 70 days. In some aspects, the IL-7 protein is administered at a dose of 1,200 μg/kg with a dosing frequency of once every 70 days. In some aspects, the IL-7 protein is administered at a dose of 1,300 μg/kg with a dosing frequency of once every 70 days. In some aspects, the IL-7 protein is administered at a dose of 1,400 μg/kg with a dosing frequency of once every 70 days. In other aspects, the IL-7 protein is administered at a dose of 1,420 μg/kg with a dosing frequency of once every 70 days. In certain aspects, the IL-7 protein is administered at a dose of 1,440 μg/kg with a dosing frequency of once every 70 days. In further aspects, the IL-7 protein is administered at a dose of 1,460 μg/kg with a dosing frequency of once every 70 days. In certain aspects, the IL-7 protein is administered at a dose of 1,480 μg/kg with a dosing frequency of once every 70 days. In some aspects, the IL-7 protein is administered at a dose of 1,500 μg/kg with a dosing frequency of once every 70 days. In further aspects, the IL-7 protein is administered at a dose of 1,600 μg/kg with a dosing frequency of once every 70 days. In some aspects, the IL-7 protein is administered at a dose of 1,700 μg/kg with a dosing frequency of once every 70 days. In some aspects, the IL-7 protein is administered at a dose of 2,000 μg/kg with a dosing frequency of once every 70 days.

In some aspects, the IL-7 protein is administered at a dose of 60 μg/kg with a dosing frequency of once every 80 days. In some aspects, the IL-7 protein is administered at a dose of 120 μg/kg with a dosing frequency of once every 80 days. In some aspects, the IL-7 protein is administered at a dose of 240 μg/kg with a dosing frequency of once every 80 days. In some aspects, the IL-7 protein is administered at a dose of 480 μg/kg with a dosing frequency of once every 80 days. In some aspects, the IL-7 protein is administered at a dose of 720 μg/kg with a dosing frequency of once every 80 days. In some aspects, the IL-7 protein is administered at a dose of 960 μg/kg with a dosing frequency of once every 80 days. In some aspects, the IL-7 protein is administered at a dose of 1,200 μg/kg with a dosing frequency of once every 80 days. In some aspects, the IL-7 protein is administered at a dose of 1,300 μg/kg with a dosing frequency of once every 80 days. In some aspects, the IL-7 protein is administered at a dose of 1,400 μg/kg with a dosing frequency of once every 80 days. In other aspects, the IL-7 protein is administered at a dose of 1,420 μg/kg with a dosing frequency of once every 80 days. In certain aspects, the IL-7 protein is administered at a dose of 1,440 μg/kg with a dosing frequency of once every 80 days. In further aspects, the IL-7 protein is administered at a dose of 1,460 μg/kg with a dosing frequency of once every 80 days. In certain aspects, the IL-7 protein is administered at a dose of 1,480 μg/kg with a dosing frequency of once every 80 days. In some aspects, the IL-7 protein is administered at a dose of 1,500 μg/kg with a dosing frequency of once every 80 days. In further aspects, the IL-7 protein is administered at a dose of 1,600 μg/kg with a dosing frequency of once every 80 days. In some aspects, the IL-7 protein is administered at a dose of 1,700 μg/kg with a dosing frequency of once every 80 days. In some aspects, the IL-7 protein is administered at a dose of 2,000 μg/kg with a dosing frequency of once every 80 days.

In some aspects, the IL-7 protein is administered at a dose of 60 μg/kg with a dosing frequency of once every 90 days. In some aspects, the IL-7 protein is administered at a dose of 120 μg/kg with a dosing frequency of once every 90 days. In some aspects, the IL-7 protein is administered at a dose of 240 μg/kg with a dosing frequency of once every 90 days. In some aspects, the IL-7 protein is administered at a dose of 480 μg/kg with a dosing frequency of once every 90 days. In some aspects, the IL-7 protein is administered at a dose of 720 μg/kg with a dosing frequency of once every 90 days. In some aspects, the IL-7 protein is administered at a dose of 960 μg/kg with a dosing frequency of once every 90 days. In some aspects, the IL-7 protein is administered at a dose of 1,200 μg/kg with a dosing frequency of once every 90 days. In some aspects, the IL-7 protein is administered at a dose of 1,300 μg/kg with a dosing frequency of once every 90 days. In some aspects, the IL-7 protein is administered at a dose of 1,400 μg/kg with a dosing frequency of once every 90 days. In other aspects, the IL-7 protein is administered at a dose of 1,420 μg/kg with a dosing frequency of once every 90 days. In certain aspects, the IL-7 protein is administered at a dose of 1,440 μg/kg with a dosing frequency of once every 90 days. In further aspects, the IL-7 protein is administered at a dose of 1,460 μg/kg with a dosing frequency of once every 90 days. In certain aspects, the IL-7 protein is administered at a dose of 1,480 μg/kg with a dosing frequency of once every 90 days. In some aspects, the IL-7 protein is administered at a dose of 1,500 μg/kg with a dosing frequency of once every 90 days. In further aspects, the IL-7 protein is administered at a dose of 1,600 μg/kg with a dosing frequency of once every 90 days. In some aspects, the IL-7 protein is administered at a dose of 1,700 μg/kg with a dosing frequency of once every 90 days. In some aspects, the IL-7 protein is administered at a dose of 2,000 μg/kg with a dosing frequency of once every 90 days.

In some aspects, the IL-7 protein is administered at a dose of 60 μg/kg with a dosing frequency of once every 100 days. In some aspects, the IL-7 protein is administered at a dose of 120 μg/kg with a dosing frequency of once every 100 days. In some aspects, the IL-7 protein is administered at a dose of 240 μg/kg with a dosing frequency of once every 100 days. In some aspects, the IL-7 protein is administered at a dose of 480 μg/kg with a dosing frequency of once every 100 days. In some aspects, the IL-7 protein is administered at a dose of 720 μg/kg with a dosing frequency of once every 100 days. In some aspects, the IL-7 protein is administered at a dose of 960 μg/kg with a dosing frequency of once every 100 days. In some aspects, the IL-7 protein is administered at a dose of 1,200 μg/kg with a dosing frequency of once every 100 days. In some aspects, the IL-7 protein is administered at a dose of 1,300 μg/kg with a dosing frequency of once every 100 days. In some aspects, the IL-7 protein is administered at a dose of 1,400 μg/kg with a dosing frequency of once every 100 days. In other aspects, the IL-7 protein is administered at a dose of 1,420 μg/kg with a dosing frequency of once every 100 days. In certain aspects, the IL-7 protein is administered at a dose of 1,440 μg/kg with a dosing frequency of once every 100 days. In further aspects, the IL-7 protein is administered at a dose of 1,460 μg/kg with a dosing frequency of once every 100 days. In certain aspects, the IL-7 protein is administered at a dose of 1,480 μg/kg with a dosing frequency of once every 100 days. In some aspects, the IL-7 protein is administered at a dose of 1,500 μg/kg with a dosing frequency of once every 100 days. In further aspects, the IL-7 protein is administered at a dose of 1,600 μg/kg with a dosing frequency of once every 100 days. In some aspects, the IL-7 protein is administered at a dose of 1,700 μg/kg with a dosing frequency of once every 100 days. In some aspects, the IL-7 protein is administered at a dose of 2,000 μg/kg with a dosing frequency of once every 100 days.

In some aspects, methods disclosed herein (e.g., administering IL-7 protein in combination with an immune checkpoint inhibitor) can be used in combination with one or more additional anti-cancer and/or immunomodulating agents. Such agents can include, for example, chemotherapy drugs, small molecule drugs, or antibodies that stimulate the immune response to a given cancer. In some aspects, the methods described herein are used in combination with a standard of care treatment (e.g., surgery, radiation, and chemotherapy). Methods described herein can also be used as a maintenance therapy, e.g., a therapy that is intended to prevent the occurrence or recurrence of tumors.

In some aspects, a method for treating a tumor disclosed herein can comprise administering an IL-7 protein in combination with one or more immuno-oncology agents, such that multiple elements of the immune pathway can be targeted. Non-limiting of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

In some aspects, an immuno-oncology agent that can be used in combination with an IL-7 protein disclosed herein comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof.

In some aspects, an immuno-oncology agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or any combination thereof.

In some aspects, a combination of an IL-7 protein and a second agent discussed herein (e.g., immune checkpoint inhibitor) can be administered concurrently as a single composition in a pharmaceutically acceptable carrier. In other aspects, a combination of an IL-7 protein and a second agent discussed herein (e.g., immune checkpoint inhibitor) can be administered concurrently as separate compositions. In further aspects, a combination of an IL-7 protein and a second agent discussed herein (e.g., immune checkpoint inhibitor) can be administered sequentially. In some aspects, an IL-7 protein is administered prior to the administration of a second agent (e.g., immune checkpoint inhibitor).

IIa. IL-7 Proteins Useful for the Disclosure

Disclosed herein are IL-7 proteins that can be used in combination with an immune checkpoint inhibitor to treat a cancer (or a tumor). In some aspects, IL-7 protein useful for the present uses can be wild-type IL-7 or modified IL-7 (i.e., not wild-type IL-7 protein) (e.g., IL-7 variant, IL-7 functional fragment, IL-7 derivative, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See ElKassar and Gress. J Immunotoxicol. 2010 March; 7(1): 1-7. In some aspects, an IL-7 protein of the present disclosure is not a wild-type IL-7 protein (i.e., comprises one or more modifications). Non-limiting examples of such modifications can include an oligopeptide and/or a half-life extending moiety. See WO 2016/200219, which is herein incorporated by reference in its entirety.

IL-7 binds to its receptor which is composed of the two chains IL-7Rα (CD127), shared with the thymic stromal lymphopoietin (TSLP) (Ziegler and Liu, 2006), and the common γ chain (CD132) for IL-2, IL-15, IL-9 and IL-21. Whereas γc is expressed by most hematopoietic cells, IL-7Rα is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/− (von Freeden-Jeffry et al., 1995), IL-7Rα−/− (Peschon et al., 1994; Maki et al., 1996), γc−/−(Malissen et al., 1997), and Jak3−/− mice (Park et al., 1995). In the absence of IL-7 signaling, mice lack T-, B-, and NK-T cells. IL-7α−/− mice (Peschon et al., 1994) have a similar but more severe phenotype than IL-7−/− mice (von Freeden-Jeffry et al., 1995), possibly because TSLP signaling is also abrogated in IL-7α−/− mice. IL-7 is required for the development of γδ cells (Maki et al., 1996) and NK-T cells (Boesteanu et al., 1997).

In some aspects, an IL-7 protein useful for the present disclosure comprises an amino acid sequence as set forth in any one of SEQ ID NOs: 1 to 6. In other aspects, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOS: 1 to 6.

In some aspects, the IL-7 protein includes a modified IL-7 or a fragment thereof, wherein the modified IL-7 or the fragment retains one or more biological activities of wild-type IL-7. In some aspects, the IL-7 protein can be derived from humans, rats, mice, monkeys, cows, or sheep.

In some aspects, the human IL-7 can have an amino acid sequence represented by SEQ ID NO: 1 (Genbank Accession No. P13232); the rat IL-7 can have an amino acid sequence represented by SEQ ID NO: 2 (Genbank Accession No. P56478); the mouse IL-7 can have an amino acid sequence represented by SEQ ID NO: 3 (Genbank Accession No. P10168); the monkey IL-7 may have an amino acid sequence represented by SEQ ID NO: 4 (Genbank Accession No. NP 001279008); the cow IL-7 can have an amino acid sequence represented by SEQ ID NO: 5 (Genbank Accession No. P26895), and the sheep IL-7 can have an amino acid sequence represented by SEQ ID NO: 6 (Genbank Accession No. Q28540).

In some aspects, an IL-7 protein useful for the present disclosure comprises an IL-7 fusion protein. In certain aspects, an IL-7 fusion protein comprises (i) an oligopeptide and (i) an IL-7 or a variant thereof. In some aspects, the oligopeptide is linked to the N-terminal region of the IL-7 or a variant thereof.

In some aspects, an oligopeptide disclosed herein consists of 1 to 10 amino acids. In certain aspects, an oligopeptide consists of at least 1, at least 2, at least 3, at least 4, at least 5, at least 6, at least 7, at least 8, at least 9, or 10 amino acids. In some aspects, one or more amino acids of an oligopeptide are selected from the group consisting of methionine, glycine, and combinations thereof. In certain aspects, an oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine. In some aspects, an oligopeptide is methionine-glycine-methionine.

In some aspects, an IL-7 fusion protein comprises (i) an IL-7 or a variant thereof, and (ii) a half-life extending moiety. In some aspects, a half-life extending moiety extends the half-life of the IL-7 or variant thereof. In some aspects, a half-life extending moiety is linked to the C-terminal region of an IL-7 or a variant thereof.

In some aspects, an IL-7 fusion protein comprises (i) IL-7 (a first domain), (ii) a second domain that includes an amino acid sequence having 1 to 10 amino acid residues consisting of methionine, glycine, or a combination thereof, e.g., MGM, and (iii) a third domain comprising a half-life extending moiety. In some aspects, the half-life extending moiety can be linked to the N-terminal or the C-terminal of the first domain or the second domain. Additionally, the IL-7 including the first domain and the second domain can be linked to both terminals of the third domain.

Non-limiting examples of half-life extending moieties include: Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, and combinations thereof.

In some aspects, a half-life extending moiety is Fc. In certain aspects, Fc is from a modified immunoglobulin in which the antibody-dependent cellular cytotoxicity (ADCC) or complement-dependent cytotoxicity (CDC) weakened due to the modification in the binding affinity with the Fc receptor and/or a complement. In some aspects, the modified immunoglobulin can be selected from the group consisting of IgG1, IgG2, IgG3, IgG4, IgA1, IgA2, IgD, IgE, and a combination thereof. In some aspects, an Fc is a hybrid Fc (“hFc” or “hyFc”), comprising a hinge region, a CH2 domain, and a CH3 domain. In certain aspects, a hinge region of a hybrid Fc disclosed herein comprises a human IgD hinge region. In certain aspects, a CH2 domain of a hybrid Fc comprises a part of human IgD CH2 domain and a part of human IgG4 CH2 domain. In certain aspects, a CH3 domain of a hybrid Fc comprises a part of human IgG4 CH3 domain. Accordingly, in some aspects, a hybrid Fc disclosed herein comprises a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, an Fc disclosed herein can be an Fc variant. As used herein, the term “Fc variant” refers to an Fc which was prepared by substituting a part of the amino acids among the Fc region or by combining the Fc regions of different kinds. The Fc region variant can prevent from being cut off at the hinge region. Specifically, in some aspects, a Fc variant comprises modifications at the 144^{th} amino acid and/or 145^{th} amino acid of SEQ ID NO: 9. In certain aspects, the 144^{th} amino acid (K) and/or the 145^{th} amino acid (K) is substituted with G or S.

In some aspects, an Fc or an Fc variant disclosed herein can be represented by the following formula: N′−(Z1)p−Y−Z2−Z3−Z4−C, wherein:

N′ comprises the N-terminus;

Z1 comprises an amino acid sequence having 5 to 9 consecutive amino acid residues from the amino acid residue at position 98 toward the N-terminal, among the amino acid residues at positions from 90 to 98 of SEQ ID NO: 7;

Y comprises an amino acid sequence having 5 to 64 consecutive amino acid residues from the amino acid residue at position 162 toward the N-terminal, among the amino acid residues at positions from 99 to 162 of SEQ ID NO: 7;

Z2 comprises an amino acid sequence having 4 to 37 consecutive amino acid residues from the amino acid residue at position 163 toward the C-terminal, among the amino acid residues at positions from 163 to 199 of SEQ ID NO: 7;

Z3 comprises an amino acid sequence having 71 to 106 consecutive amino acid residues from the amino acid residue at position 220 toward the N-terminal, among the amino acid residues at positions from 115 to 220 of SEQ ID NO: 8; and

Z4 comprises an amino acid sequence having 80 to 107 consecutive amino acid residues from the amino acid residue at position 221 toward the C-terminal, among the amino acid residues at positions from 221 to 327 of SEQ ID NO: 8.

In some aspects, a Fc region disclosed herein can include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). In some aspects, the Fc region can include the amino acid sequence of SEQ ID NO: 14 (a non-lytic mouse Fc).

Other non-limiting examples of Fc regions that can be used with the present disclosure are described in U.S. Pat. No. 7,867,491, which is herein incorporated by reference in its entirety.

In some aspects, an IL-7 fusion protein disclosed herein comprises both an oligopeptide and a half-life extending moiety.

In some aspects, an IL-7 protein can be fused to albumin, a variant, or a fragment thereof. Examples of the IL-7-albumin fusion protein can be found at International Application Publication No. WO 2011/124718 A1. In some aspects, an IL-7 protein is fused to a pre-pro-B cell Growth Stimulating Factor (PPBSF), optionally by a flexible linker. See US 2002/0058791A1. In other aspects, an IL-7 protein useful for the disclosure is an IL-7 conformer that has a particular three dimensional structure. See US 2005/0249701 A1. In some aspects, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2. In some aspects, an IL-7 protein that does not contain potential T-cell epitopes (thereby to reduce anti-IL-7 T-cell responses) can also be used for the present disclosure. See US 2006/0141581 A1. In other aspects, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood,vol. 114 (4), 816, 23 Jul. 2009, Kang et al., J. Virol. Doi:10.1128/JVI.02768-15, Martin et al., Blood, vol. 121 (22), 4484, May 30, 2013, McBride et al., Acta Oncologica, 34:3, 447-451, Jul. 8, 2009, and Xu et al., Cancer Science, 109: 279-288, 2018, which are incorporated herein by reference in their entireties.

The present disclosure is directed to a method for treating a tumor (or a cancer) in a subject in need thereof, comprising administering to the subject an effective amount of an interleukin-7 (IL-7) protein in combination with an effective amount of an immune checkpoint inhibitor. Non-limiting examples of immune checkpoint inhibitors that can be used with the current methods include an anti-PD-1 antibody, anti-PD-L1 antibody, anti-CTLA-4 antibody, and combinations thereof.

In some aspects, an oligopeptide disclosed herein is directly linked to the N-terminal region of IL-7 or a variant thereof. In other aspects, an oligopeptide is linked to the N-terminal region via a linker. In some aspects, a half-life extending moiety disclosed herein is directly linked to the C-terminal region of IL-7 or a variant thereof. In certain aspects, a half-life extending moiety is linked to the C-terminal region via a linker. In some aspects, a linker is a peptide linker. In certain aspects, a peptide linker comprises a peptide of 10 to 20 amino acid residues consisting of Gly and Ser residues. In some aspects, a linker is an albumin linker. In some aspects, a linker is a chemical bond. In certain aspects, a chemical bond comprises a disulfide bond, a diamine bond, a sulfide-amine bond, a carboxy-amine bond, an ester bond, a covalent bond, or combinations thereof. When the linker is a peptide linker, in some aspects, the connection can occur in any linking region. They may be coupled using a crosslinking agent known in the art. In some aspects, examples of the crosslinking agent can include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

In some aspects, an IL-7 (or variant thereof) portion of IL-7 fusion protein disclosed herein comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 15-20. In certain aspects, an IL-7 (or variant thereof) portion of IL-7 fusion protein disclosed herein comprises the amino acid sequence set forth in SEQ ID NOs: 15-20.

In some aspects, an IL-7 fusion protein comprises: a first domain including a polypeptide having the activity of IL-7 or a similar activity thereof; a second domain comprising an amino acid sequence having 1 to 10 amino acid residues consisting of methionine, glycine, or a combination thereof; and a third domain, which is an Fc region of modified immunoglobulin, coupled to the C-terminal of the first domain.

In some aspects, an IL-7 fusion protein that can be used with the present methods comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 21-25. In certain aspects, an IL-7 fusion protein of the present disclosure comprises the amino acid sequence set forth in SEQ ID NOs: 21-25. In further aspects, an IL-7 fusion protein disclosed herein comprises the amino acid sequence set forth in SEQ ID NOs: 26 and 27.

In some aspects, an IL-7 protein useful for the present disclosure can increase absolute lymphocyte counts in a subject when administered to the subject. In certain aspects, the subject suffers from a disease or disorder described herein (e.g., cancer). In other aspects, the subject is a healthy individual (e.g., does not suffer from a disease or disorder described herein, e.g., cancer). In certain aspects, the absolute lymphocyte count is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% or more, compared to a reference (e.g., corresponding level in a subject that did not receive the IL-7 protein).

In some aspects, an IL-7 protein disclosed herein can increase T cell proliferation (e.g., CD8⁺ T cells) in a subject. In certain aspects, the increase in T cell proliferation occurs in the periphery (e.g., not within the tumor). In certain aspects, T cell proliferation is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% or more, compared to a reference (e.g., corresponding level in a subject that did not receive the IL-7 protein). In certain aspects, T cells (e.g., CD8⁺ T cells) that proliferate in response to the IL-7 administration express one or more of the following markers: Eomesodermin (Eomes), granzyme B, CXCR3, IFN-γ, or combinations thereof.

In some aspects, an IL-7 protein of the present disclosure can increase the recruitment of effector T cells (e.g., cytotoxic CD8⁺ T lymphocytes) to the tumor. In certain aspects, recruitment of effector T cells to the tumor is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% or more, compared to a reference (e.g., corresponding level in a subject that did not receive the IL-7 protein).

In some aspects, an IL-7 protein of the present disclosure can decrease the number and/or percentage of myeloid-derived suppressor cells (MDSCs) in the tumor of a subject. In certain aspects, the number and/or percentage of MDSCs in the tumor is decreased by at least about 5%, at least about 10%, at least about 20%, at least about 30%, at least about 40%, at least about 50%, at least about 60%, at least about 70%, at least about 80%, at least about 90%, or about 100% compared to a reference (e.g., corresponding level in a subject that did not receive the IL-7 protein).

In some aspects, an IL-7 protein that can be used with the present disclosure can increase the ratio of CD8⁺ TILs to MDSCs in a tumor when administered to a subject. In certain aspects, the ratio of CD8⁺ TILs to MDSCs is increased by at least about 5%, at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration compared to a reference (e.g., corresponding level in a subject that did not receive the IL-7 protein).

IIb. PD-1 Antagonists

In some aspects, the present disclosure provides a method of treating a tumor in a subject in need thereof, comprising administering to the subject an effective amount of an IL-7 protein in combination with an effective amount of an antagonist of the PD-1 pathway (“PD-1 antagonist”). As used herein, the term “PD-1 antagonist” can be used interchangeably with the term “PD-1 pathway inhibitor” and includes, but is not limited to, PD-1 binding agents, PD-L1 binding agent, and PD-L2 binding agents. PD-1 binding agents include antibodies that specifically bind to PD-1. PD-L1 and PD-L2 binding agents include antibodies that specifically bind to PD-L1 and/or PD-L2, as well as soluble PD-1 polypeptides that bind to PD-L1 and/or PD-L2.

### Anti-PD-1 Antibodies

In some aspects, a PD-1 antagonist that can be used with the present disclosure is an anti-PD-1 antibody. Antibodies (e.g., human antibodies) that bind specifically to PD-1 with high affinity have been disclosed in U.S. Pat. Nos. 8,008,449 and 8,779,105, each of which is hereby incorporated by reference. Other anti-PD-1 mAbs have been described in, for example, U.S. Pat. Nos. 6,808,710, 7,488,802, 8,168,757 and 8,354,509, and PCT Publication No. WO 2012/145493, each of which is hereby incorporated by reference. Each of the anti-PD-1 HuMAbs disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-y production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 antibodies useful for the present invention include mAbs that bind specifically to human PD-1 and exhibit at least one, preferably at least five, of the preceding characteristics.

In some aspects, the anti-PD-1 antibody is nivolumab. Nivolumab (also known as “OPDIVO®”; 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56, each of which is hereby incorporated by reference). In some aspects, the anti-PD-1 antibody or fragment thereof cross-competes with nivolumab. In other aspects, the anti-PD-1 antibody or fragment thereof binds to the same epitope as nivolumab. In certain aspects, the anti-PD-1 antibody has the same CDRs as nivolumab. In some aspects, the anti-PD-1 antibody (e.g., nivolumab) is administered to the subject (e.g., in combination with an IL-7 protein disclosed herein) at a flat dose of about 240 mg every two weeks or about 480 mg every four weeks. In certain aspects, the anti-PD-1 antibody (e.g., nivolumab) is administered at a weight-based dose of about 3 mg/kg every two weeks.

Anti-PD-1 antibodies (or VH and/or VL domains derived therefrom) suitable for use in the invention can be generated using methods well known in the art. Alternatively, art recognized anti-PD-1 antibodies can be used. For example, monoclonal antibodies 5C4 (referred to herein as Nivolumab or BMS-936558), 17D8, 2D3, 4H1, 4A11, 7D3, and 5F4, described in WO 2006/121168, the teachings of which are hereby incorporated by reference, can be used. Other known PD-1 antibodies include lambrolizumab (MK-3475) described in WO 2008/156712, and AMP-514 described in WO 2012/145493, the teachings of which are hereby incorporated by reference. Further known anti-PD-1 antibodies and other PD-1 inhibitors include those described in WO 2009/014708, WO 03/099196, WO 2009/114335 and WO 2011/161699, the teachings of which are hereby incorporated by reference. Another known anti-PD-1 antibody is pidilizumab (CT-011). Antibodies or antigen binding fragments thereof that compete with any of these antibodies or inhibitors for binding to PD-1 also can be used.

In some aspects, the anti-PD-1 antibody or antigen binding fragment thereof cross-competes with pembrolizumab. In some aspects, the anti-PD-1 antibody or antigen binding fragment thereof binds to the same epitope as pembrolizumab. In certain aspects, the anti-PD-1 antibody or antigen binding fragment thereof has the same CDRs as pembrolizumab. In another aspect, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also worldwideweb.cancer.gov/drugdictionary?cdrid=695789 (last accessed: May 25, 2017), each of which is hereby incorporated by reference. Pembrolizumab has been approved by the FDA for the treatment of relapsed or refractory melanoma. In some aspects, the anti-PD-1 antibody (e.g., pembrolizumab) is administered to the subject (e.g., in combination with an IL-7 protein disclosed herein) at a flat dose of about 200 mg every three weeks. In certain aspects, the anti-PD-1 antibody (e.g., pembrolizumab) is administered at a weight-based dose of about 2 mg/kg every three weeks.

In other aspects, the anti-PD-1 antibody or antigen binding fragment thereof cross-competes with MEDI0608. In still other aspects, the anti-PD-1 antibody or antigen binding fragment thereof binds to the same epitope as MEDI0608. In certain aspects, the anti-PD-1 antibody has the same CDRs as MEDI0608. In other aspects, the anti-PD-1 antibody is MEDI0608 (formerly AMP-514), which is a monoclonal antibody. MEDI0608 is described, for example, in U.S. Pat. No. 8,609,089 or in worldwideweb.cancer.gov/drugdictionary?cdrid=756047 (last accessed May 25, 2017), each of which is hereby incorporated by reference.

In other aspects, the anti-PD-1 antibody or antigen binding fragment thereof cross-competes with BGB-A317. In some aspects, the anti-PD-1 antibody or antigen binding fragment thereof binds the same epitope as BGB-A317. In certain aspects, the anti-PD-1 antibody or antigen binding fragment thereof has the same CDRs as BGB-A317. In certain aspects, the anti-PD-1 antibody or antigen binding fragment thereof is BGB-A317, which is a humanized monoclonal antibody. BGB-A317 is described in U.S. Publ. No. 2015/0079109, which is hereby incorporated by reference.

In some aspects, antibodies or antigen binding fragments thereof that cross-compete for binding to human PD-1 with, or bind to the same epitope region of human PD-1 as, nivolumab are mAbs. For administration to human subjects, these cross-competing antibodies can be chimeric antibodies, or humanized or human antibodies. Such chimeric, humanized or human mAbs can be prepared and isolated by methods well known in the art.

Anti-PD-1 antibodies or antigen binding fragments thereof suitable for use in the present disclosure are antibodies that bind to PD-1 with high specificity and affinity, block the binding of PD-L 1 and or PD-L2, and inhibit the immunosuppressive effect of the PD-1 signaling pathway. In certain aspects, the anti-PD-1 antibody, or antigen-binding portion thereof, cross-competes with nivolumab for binding to human PD-1. In other aspects, the anti-PD-1 antibody, or antigen-binding portion thereof, is a chimeric, humanized or human monoclonal antibody or a portion thereof In certain aspects, the antibody is a humanized antibody. In other aspects, the antibody is a human antibody. Antibodies of an IgG1, IgG2, IgG3 or IgG4 isotype can be used.

In certain aspects, the anti-PD-1 antibody or antigen binding fragment thereof comprises a heavy chain constant region which is of a human IgG1 or IgG4 isotype. In certain other aspects, the sequence of the IgG4 heavy chain constant region of the anti-PD-1 antibody or antigen binding fragment thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other aspects, the antibody comprises a light chain constant region which is a human kappa or lambda constant region. In other aspects, the anti-PD-1 antibody, or antigen binding fragment thereof, is a mAb or an antigen-binding portion thereof. In certain aspects of any of the therapeutic methods described herein comprising administration of an anti-PD-1 antibody, the anti-PD-1 antibody is nivolumab. In other aspects, the anti-PD-1 antibody is pembrolizumab. In other aspects, the anti-PD-1 antibody is chosen from the human antibodies 17D8, 2D3, 4H1, 4A11, 7D3 and 5F4 described in U.S. Pat. No. 8,008,449, which is hereby incorporated by reference. In still other aspects, the anti-PD-1 antibody is MEDI0608 (formerly AMP-514), AMP-224, or Pidilizumab (CT-011).

### Anti-PD-L1 Antibodies

In some aspects, a PD-1 antagonist that can be used with the present disclosure is an anti-PD-L1 antibody. Anti-human-PD-L1 antibodies (or VH and/or VL domains derived therefrom) suitable for use in the invention can be generated using methods well known in the art. Alternatively, art recognized anti-PD-L1 antibodies can be used. For example, human anti-PD-L1 antibodies disclosed in U.S. Pat. No. 7,943,743, the contents of which are hereby incorporated by reference, can be used. Such anti-PD-L1 antibodies include 3G10, 12A4 (also referred to as BMS-936559), 10A5, 5F8, 10H10, 1B12, 7H1, 11E6, 12B7, and 13G4. Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, the teachings of which also are hereby incorporated by reference. Other examples of an anti-PD-L1 antibody include atezolizumab (TECENTRIQ®; RG7446), or durvalumab (IMFINZI®; MEDI4736). Antibodies or antigen binding fragments thereof that compete with any of these art-recognized antibodies or inhibitors for binding to PD-L1 also can be used. In some aspects, an anti-PD-L1 antibody (e.g., atezolizumab) is administered to the subject (e.g., in combination with an IL-7 protein disclosed herein) at a dose of about 1200 mg every three weeks. In some aspects, an anti-PD-L1 antibody (e.g., durvalumab) is administered (e.g., in combination with an IL-7 protein disclosed herein) at a dose of about 10 mg/kg every two weeks.

In certain aspects, the anti-PD-L1 antibody is BMS-936559 (formerly 12A4 or MDX-1105) (see, e.g., U.S. Pat. No. 7,943,743; WO 2013/173223, both of which are hereby incorporated by reference). In other aspects, the anti-PD-L1 antibody is MPDL3280A (also known as RG7446 and atezolizumab) (see, e.g., Herbst et al. 2013 J Clin Oncol 31(suppl):3000; U.S. Pat. No. 8,217,149, both of which are hereby incorporated by reference), MEDI4736 (Khleif, 2013, In: Proceedings from the European Cancer Congress 2013; Sep. 27-Oct. 1, 2013; Amsterdam, The Netherlands. Abstract 802, which is hereby incorporated by reference), or MSB0010718C (also called Avelumab; see US 2014/0341917, which is hereby incorporated by reference). In certain aspects, antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art. In some aspects, an anti-PD-L1 antibody (e.g., avelumab) is administered to the subject (e.g., in combination with an IL-7 protein disclosed herein) at a dose of about 800 mg every two weeks.

IIc. CTLA-4 Antagonists

In some aspects, the present disclosure also provides a method of treating a tumor in a subject in need thereof, comprising administering to the subject an effective amount of an IL-7 protein in combination with an effective amount of an antagonist of the CTLA-4 pathway (“CTLA-4 antagonist”). In some aspects, a CTLA-4 antagonist is an anti-CTLA-4 antibody.

HuMAbs that bind specifically to CTLA-4 with high affinity have been disclosed in U.S. Pat. Nos. 6,984,720 and 7,605,238, each of which is hereby incorporated by reference. Other anti-CTLA-4 mAbs have been described in, for example, U.S. Pat. Nos. 5,977,318, 6,051,227, 6,682,736, and 7,034,121, each of which is hereby incorporated by reference. The anti-CTLA-4 HuMAbs disclosed in U.S. Pat. Nos. 6,984,720 and 7,605,238, both of which are hereby incorporated by reference, have been demonstrated to exhibit one or more of the following characteristics: (a) binds specifically to human CTLA-4 with a binding affinity reflected by an equilibrium association constant (Kₐ) of at least about 10⁷M⁻¹, or about 10⁹ M⁻¹, or about 10¹⁰ M⁻¹ to 10¹¹M⁻¹ or higher, as determined by Biacore analysis; (b) a kinetic association constant (1) of at least about 10³, about 10⁴, or about 10⁵ m⁻¹ s⁻¹; (c) a kinetic disassociation constant (k_{d}) of at least about 10³, about 10⁴, or about 10⁵ m⁻¹ 5⁻¹; and (d) inhibits the binding of CTLA-4 to B7-1 (CD80) and B7-2 (CD86). Anti-CTLA-4 antibodies useful for the present invention include mAbs that bind specifically to human CTLA-4 and exhibit at least one, at least two, or at least three of the preceding characteristics. An exemplary clinical anti-CTLA-4 antibody is the human mAb 10D1 (now known as ipilimumab and marketed as YERVOY®) as disclosed in U.S. Pat. No. 6,984,720, which is hereby incorporated by reference. Ipilimumab is an anti-CTLA-4 antibody for use in the methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma. In some aspects, an anti-CTLA-4 antibody (e.g., ipilimumab) is administered to the subject (e.g., in combination with an IL-7 protein disclosed herein) at a dose of about 3 mg/kg every 3 weeks (e.g., to treat unresectable or metastatic melanoma). In some aspects, an anti-CTLA-4 antibody (e.g., ipilimumab) is administered to the subject (e.g., in combination with an IL-7 protein disclosed herein) at a dose of about 10 mg/kg every three weeks for four doses, followed by 10 mg/kg every twelve weeks for up to three years (e.g., to treat adjuvant melanoma).

Another anti-CTLA-4 antibody useful for the present methods is tremelimumab (also known as ticilimumab and CP-675,206). Tremelimumab is human IgG2 monoclonal anti-CTLA-4 antibody. Tremelimumab is described in WO/2012/122444, U.S. Publ. No. 2012/263677, and WO Publ. No. 2007/113648 A2, each of which is hereby incorporated by reference. Other non-limiting examples of anti-CTLA-4 antibodies that are useful for the present disclosure include: MK-1308 (Merck) and AGEN-1884 (Agenus Inc.; see WO 2016/196237).

Anti-CTLA-4 antibodies useful for the present disclosure also include isolated antibodies that bind specifically to human CTLA-4 and cross-compete for binding to human CTLA-4 with ipilimumab, tremelimumab, MK-1308, or AGEN-1884, or bind to the same epitope region of human CTLA-4 as ipilimumab, tremelimumab, MK-1308, or AGEN-1884. In certain aspects, the antibodies that cross-compete for binding to human CTLA-4 with, or bind to the same epitope region of human CTLA-4 as does ipilimumab, tremelimumab, MK-1308, or AGEN-18844, are antibodies comprising a heavy chain of the human IgG1 isotype. For administration to human subjects, these cross-competing antibodies are chimeric antibodies, or humanized or human antibodies. Antigen-binding portions of the above antibodies, such as Fab, F(ab')₂, Fd or Fv fragments, can also be used with the present methods.

### III. Nucleic Acids, Vectors, Host Cells

Further aspect described herein pertains to one or more nucleic acid molecules that encode a therapeutic agent described herein (e.g., an IL-7 protein). The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In certain aspects, the nucleic acid is a cDNA molecule. Nucleic acids described herein can be obtained using standard molecular biology techniques known in the art.

Certain nucleic acid molecules disclosed herein are those encoding an IL-7 protein (e.g., disclosed herein). Exemplary nucleic acid sequences encoding an IL-7 protein disclosed herein are set forth in SEQ ID NOs: 29-39.

In some aspects, the present disclosure provides a vector comprising an isolated nucleic acid molecule encoding a therapeutic agent disclosed herein (e.g., an IL-7 protein). In some aspects, a vector can be used for gene therapy.

When used as a gene therapy (e.g., in humans), a nucleic acid encoding a therapeutic agent disclosed herein (e.g., an IL-7 protein) can be administered at a dosage in the range of 0.1 mg to 200 mg. In certain aspects, the dosage is in the range of 0.6 mg to 100 mg. In further aspects, the dosage is in the range of 1.2 mg to 50 mg.

Suitable vectors for the disclosure include expression vectors, viral vectors, and plasmid vectors. In some aspects, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In other aspects, a vector is derived from a lentivirus. Examples of the lentiviral vectors are disclosed in WO09931251, WO9712622, WO9817815, WO9817816, and WO9818934, each which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

Also encompassed by the present disclosure is a method for making a therapeutic agent disclosed herein (e.g., an IL-7 protein). In some aspects, such a method can comprise expressing the therapeutic agent (e.g., an IL-7 protein) in a cell comprising a nucleic acid molecule encoding the therapeutic agent, e.g., SEQ ID NOs: 29-39. Additional details regarding the method for making an IL-7 protein disclosed herein are provided, e.g., in WO 2016/200219, which is herein incorporated by reference in its entirety. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### IV. Pharmaceutical Compositions

Further provided herein are compositions comprising one or more therapeutic agents (e.g., an IL-7 protein and/or an immune checkpoint inhibitor) having the desired degree of purity in a physiologically acceptable carrier, excipient or stabilizer (Remington's Pharmaceutical Sciences (1990) Mack Publishing Co., Easton, Pa.). In some aspects, a composition disclosed herein comprises an IL-7 protein or an immune checkpoint inhibitor. As disclosed herein, such compositions can be used in combination (e.g., a first composition comprising an IL-7 protein and a second composition comprising an immune checkpoint inhibitor). In other aspects, a composition disclosed herein can comprise both an IL-7 protein and an immune checkpoint inhibitor.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a composition disclosed herein (e.g., comprising an IL-7 protein or an immune checkpoint inhibitor) comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a composition disclosed herein can also comprise a stabilizing agent. Non-limiting examples of stabilizing agents that can be used with the present disclosure include: sucrose, trehalose, raffinose, arginine, or combinations thereof.

In some aspects, a composition disclosed herein comprises a surfactant. In certain aspects, the surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, a composition comprising an IL-7 protein can be formulated using the same formulation of an immune checkpoint inhibitor disclosed herein (e.g., which is to be used in combination with the IL-7 protein). In other aspects, an IL-7 protein and an immune checkpoint inhibitor are formulated using different formulations.

In some aspects, an IL-7 protein disclosed herein is formulated in a composition comprising (a) a basal buffer, (b) a sugar, and (c) a surfactant. In certain aspects, the basal buffer comprises histidine-acetate or sodium citrate. In some aspects, the basal buffer is at a concentration of about 10 to about 50 nM. In some aspects, a sugar comprises sucrose, trehalose, dextrose, or combinations thereof. In some aspects, the sugar is present at a concentration of about 2.5 to about 5.0 w/v %. In further aspects, the surfactant is selected from polysorbate, polyoxyethylene alkyl ether, polyoxyethylene stearate, alkyl sulfates, polyvinyl pyridone, poloxamer, or combinations thereof. In some embodiments, the surfactant is at a concentration of about 0.05% to about 6.0 w/v %.

In some aspects, the composition in which IL-7 is formulated further comprises an amino acid. In certain embodiments, the amino acid is selected from arginine, glutamate, glycine, histidine, or combinations thereof. In certain aspects, the composition further comprises a sugar alcohol. Non-limiting examples of sugar alcohol includes: sorbitol, xylitol, maltitol, mannitol, or combinations thereof.

In some aspects, an IL-7 protein disclosed herein is formulated in a composition comprising the following: (a) sodium citrate (e.g., about 20 mM), (b) sucrose (e.g., about 5%), (c) sorbitol (e.g., about 1.5%), and (d) Tween 80 (e.g., about 0.05%).

In some aspects, an IL-7 protein of the present disclosure is formulated as described in WO 2017/078385 A1, which is incorporated herein in its entirety.

In some aspects, a composition that can be used with the IL-7 protein disclosed herein comprises: (i) nivolumab (OPDIVO®) (e.g., about 10 mg), (ii) mannitol (e.g., about 30 mg), (iii) pentetic acid (e.g., about 0.008 mg), (iv) polysorbate 80 (e.g., about 0.2 mg), (v) sodium chloride (e.g., about 2.92 mg), and (vi) sodium citrate dehydrate (e.g., about 5.88 mg). In certain aspects, the composition can further comprise hydrochloric acid and/or sodium hydroxide to adjust the pH of the composition to about 6.

In some aspects, a composition that can be used with the IL-7 protein disclosed herein comprises: (i) pembrolizumab (KEYTRUIDA®) (e.g., about 25 mg), (ii) L-histidine (e.g., about 1.55 mg), (iii) polysorbate 80 (e.g., about 0.2 mg), and (iv) sucrose (e.g., about 70 mg). In certain aspects, the composition can also comprise hydrochloric acid and/or sodium hydroxide to adjust the pH to about 5.5.

In some aspects, a composition that can be used with the IL-7 protein disclosed herein comprises: (i) atezolizumab)(TECENTRIQ®) (e.g., about 60 mg), (ii) glacial acetic acid (e.g., about 16.5 mg), (iii) L-histidine (e.g., about 62 mg), (iv) sucrose (e.g., about 821.6 mg), and (v) polysorbate 20 (e.g., about 8 mg). In certain aspects, the composition comprises hydrochloric acid and/or sodium hydroxide to adjust the pH to about 5.8.

In some aspects, a composition that can be used with the IL-7 protein disclosed herein comprises: (i) durvalumab (IMIFINZI®) (e.g., about 50 mg), (ii) L-histidine (e.g., about 2 mg), (iii) L-histidine hydrochloride monohydrate (e.g., about 2.7 mg), (iv) α,α-trehalose dihydrate (e.g., about 104 mg), and (v) polysorbate 80 (e.g., about 0.2 mg).

In some aspects, a composition that can be used with the IL-7 protein disclosed herein comprises (i) ipilimumab (YERVOY®) (e.g., 5 mg), (ii) diethylene triamine pentaacetic acid (DTPA) (e.g., about 0.04 mg), (iii) mannitol (e.g., about 10 mg), (iv) polysorbate 80 (vegetable origin) (e.g., about 0.1 mg), (v) sodium chloride (e.g., about 5.85 mg), and (vi) tris hydrochloride (e.g., about 3.15 mg).

In some aspects, a composition that can be used with the IL-7 protein disclosed herein comprises (i) avelumab (BAVENCIO®) (e.g., about 20 mg), (ii) D-mannitol (e.g., about 51 mg), (iii) glacial acetic acid (e.g., about 0.6 mg), (iv) polysorbate 20 (e.g., about 0.5 mg), and (v) sodium hydroxide (e.g., about 0.3 mg).

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

An antibody or antigen-binding portion thereof described herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in one aspect, have diameters of less than 50 microns, in one aspect less than 10 microns.

A therapeutic agent disclosed herein (e.g., an IL-7 protein) can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, each of which is herein incorporated by reference in its entirety.

In certain aspects, a pharmaceutical composition comprising a therapeutic agent described herein (e.g., an IL-7 protein or an immune checkpoint inhibitor) is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in one aspect, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

The compositions to be used for in vivo administration can be sterile. This is readily accomplished by filtration through, e.g., sterile filtration membranes.

The following examples are merely illustrative and should not be construed as limiting the scope of this disclosure in any way as many variations and equivalents will become apparent to those skilled in the art upon reading the present disclosure.

### EXAMPLES

### Example 1 Effect of IL-7 Protein and PD-1 Pathway Inhibitor Combination Treatment on Tumor Volume

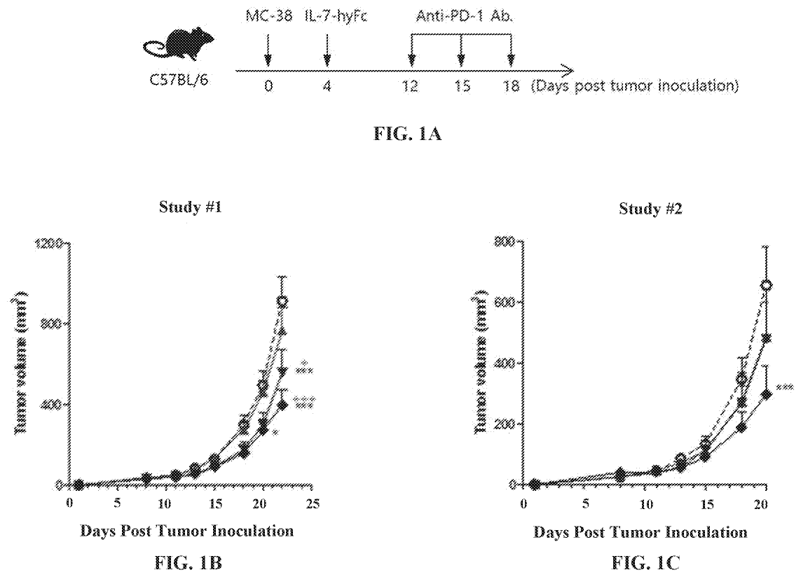

To assess the effect of IL-7 protein in combination with a PD-1 pathway inhibitor on tumor volume, a colon adenocarcinoma animal model was used. Briefly, MC-38 colon adenocarcinoma tumor cells (1×10⁵, subcutaneously) were transplanted into each C57BL/6 mice. On day 4 post tumor inoculation, the animals received a subcutaneous administration of IL-7 protein (1.25 mpk or 25 μg/mouse) or IL-7-formulating buffer. See FIG. 1A. Then, on days 12, 15, and 18 post tumor inoculation, anti-PD-1 antibody (5 mpk or 100 μg/mouse) or isotype control antibody was intraperitoneally administered to the animals. Tumor volume was measured on days 8, 11, 13, 15, 18, and 20 post tumor inoculation. FIG. 1A provides a graphical depiction of the dosing schedule and Table 1 (below) provides the different treatment groups.

TABLE 1
Treatment Groups

| Group | Treatment Regimen |
|---|---|
| Control | IL-7-formulating buffer only |
| IL-7 Protein | IL-7 protein + PBS |
| Anti-PD-1 Antibody | IL-7-formulating buffer + anti-PD-1 antibody |
| Combination | IL-7 protein + anti-PD-1 antibody |

FIGS. 1B and 1C provide the results from two separate studies. As shown, animals treated with the combination therapy (IL-7 protein+anti-PD-1 antibody) had significantly reduced tumor volume compared to not only the control animals (i.e., received IL-7-formulating buffer only) but also animals treated with the IL-7 protein or the anti-PD-1 antibody alone.

### Example 2 Effect of IL-7 Protein and PD-1 Pathway Inhibitor Combination Treatment on Tumor-Infiltrating Lymphocytes

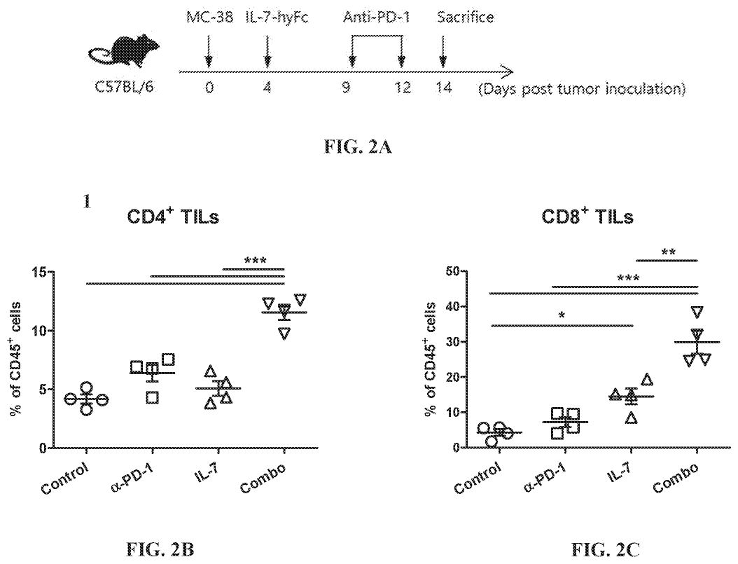

To further evaluate the anti-tumor effects of IL-7 protein in combination with a PD-1 pathway inhibitor, MC-38 colon adenocarcinoma tumor cells (1×10⁵, subcutaneously) were again transplanted into C57BL/6 mice. On day 4 post tumor inoculation, the animals were treated with a single dose of IL-7 protein (1.25 mpk or 25 μg/mouse, subcutaneously) or IL-7-formulating buffer. See FIG. 2A. Then, on days 9 and 12 post tumor inoculation, animals were treated with anti-PD-1 antibody (5 mpk or 100 μg/mouse, intraperitoneally) or isotype control antibody. Animals were sacrificed on day 14 post tumor inoculation, and the tumor-infiltrating lymphocytes in the different animals were analyzed with flow cytometry.

As shown in FIG. 2B, in animals treated with either anti-PD-1 antibody or IL-7 protein alone, approximately 5-7% of the CD45⁺ cells in the tumors of the animals were CD4⁺ TILs. This percentage was similar to what was observed in the control animals. However, in animals treated with the combination treatment (IL-7 protein+anti-PD-1 antibody), there was a significant in increase in the number of CD4⁺ TILs in the tumors (approximately 10-12% of the CD45⁺ cells in the tumors). As shown in FIG. 2C, in case of CD8⁺ TILs, treatment of IL-7 protein alone moderately increased the number of CD8⁺ TILs compared to both the control animals and animals treated with anti-PD-1 antibody alone. When animals were treated with both IL-7 protein and anti-PD-1 antibody, there was even a further increase in the number of CD8⁺ TILs among the CD45⁺ cells.

Collectively, the above results from both Examples 1 and 2 demonstrate that a treatment regimen of IL-7 protein in combination with PD-1 pathway inhibitor (e.g., anti-PD-1 antibody) can effectively treat cancer.

### Example 3 Effect of Triple Combination of Cyclophosphamide (CPA), IL-7 Protein, and PD-1 Pathway Inhibitor on Tumor Volume and Survival

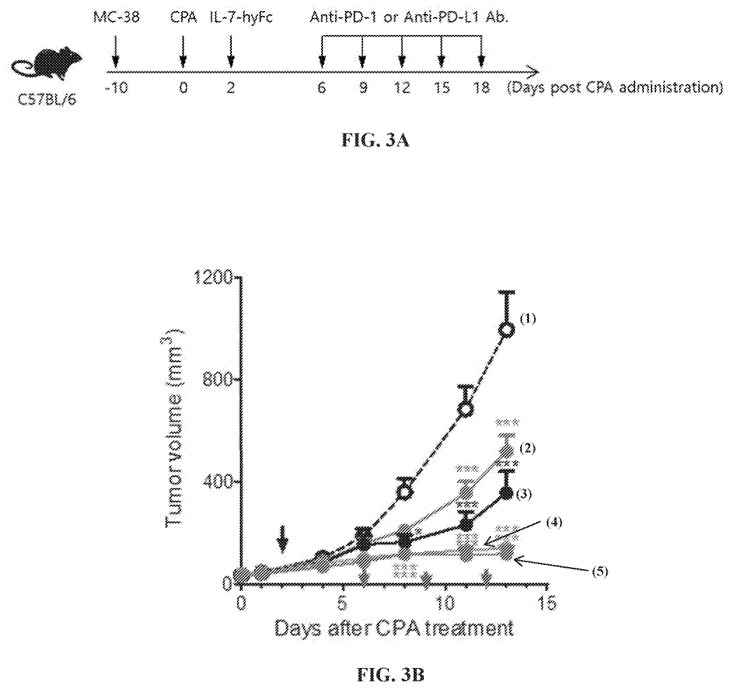

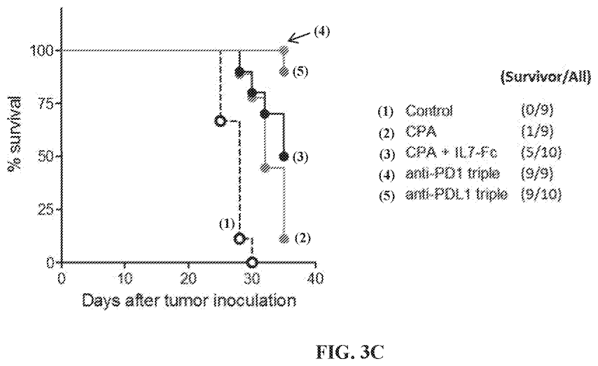

Next, the anti-tumor effects of IL-7 protein and PD-1 pathway inhibitor treatment in combination with a chemotherapeutic agent (e.g. CPA) were assessed in the colon adenocarcinoma animal model. Briefly, MC-38 colon adenocarcinoma tumor cells (1×10⁵, subcutaneously) were transplanted into C57BL/6 mice. Then, 10 days after tumor inoculation, the animals received a single dose of CPA (100 mpk or 2 mg/mouse) or PBS intraperitoneally. At day 2 post CPA administration, the animals received a subcutaneous administration of IL-7 protein (10 mpk or 200 μg/mouse) or IL-7-formulating buffer. Starting on day 6 post CPA administration, anti-PD-1 antibody (5 mpk or 100 μg/mouse), anti-PD-L1-antibody (5 mpk or 100 μg/mouse), or an isotype control antibody was intraperitoneally administered to the animals every 3 days for a total of 5 doses (i.e., days 6, 9, 12, 15, and 18 post CPA induction). See FIG. 3A. Tumor volume was measured on days 0, 1, 4, 6, 8, 11, 13, 15, 18, and 20 post CPA induction.

As shown in FIG. 3B, animals treated with CPA and IL-7 protein (“3”) had marked reduction in tumor volume compared to the animals treated with PBS (“1”) or CPA alone (“2”). Addition of either anti-PD1 antibody or anti-PD-L1 antibody to the CPA and IL-7 protein further reduced tumor volume in the animals (“4” and “5,” respective). As shown in FIG. 3C, the increased reduction in tumor volume correlated with increased survival.

The above result demonstrates that the IL-7 protein and PD-1 pathway inhibitor combination treatment can be effectively used in combination with other anti-cancer agents, such as cyclophosphamide.

### Example 4 Effect of IL-7 Protein and PD-1 Pathway Inhibitor Combination Treatment on Tumor Volume in Thymectomy-Induced Lymphopenia

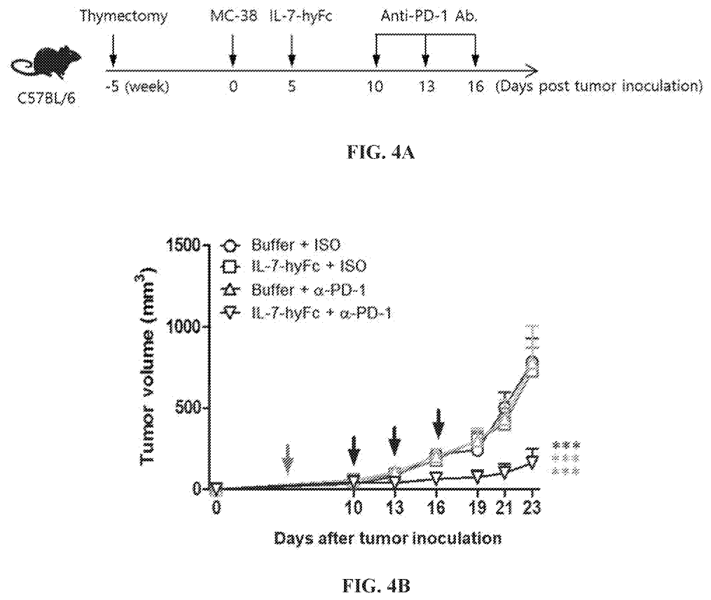

As discussed supra, many anticancer agents (e.g., chemotherapy or radiation therapy) can cause lymphopenia in a cancer subject. Therefore, to assess the anti-tumor effects of IL-7 protein in combination with PD-1 pathway inhibitor in a lymphopenic condition, thymectomized mice were used. Briefly, C57BL/6 mice were anesthetized and fixed onto a dissecting board. Using a rubber band, the airway of the mouse was opened by lifting the head backward. A rolled-up tissue pad was placed under the mouse's shoulders to aid in pushing the heart and thymus forward for easier access. For sterilizing, neck and upper chest area of the mouse were swabbed with 70% ethanol. Over the suprasternal notch, a midline longitudinal skin was incised 1.5 to 2 cm down the chest. A scissor was inserted under the sternum to cut first rib. Chest was opened by extending the forceps. After strap muscles were rent apart, the thymus was carefully gripped and then taken out from the chest. The midline longitudinal skin was closed quickly using applier and clips which are used to animal skin suture. The time from cutting first rib to skin closing was less than 1 minute. The animals were allowed to recover from the surgery for approximately 5 weeks. Then, the animals were inoculated with MC-38 colon adenocarcinoma tumor cells (1×10⁵, subcutaneously). See FIG. 4A. On day 5 post tumor inoculation, the animals received a subcutaneous administration of IL-7 protein (1.25 mpk or 25 μg/mouse) or IL-7-formulating buffer. Anti-PD-1 antibody (5 mpk or 100 pg/mouse) or isotype control antibody was administered to the animals on days 10, 13, and 16 post tumor inoculation. Tumor volume was measured on days 10, 13, 16, 19, 21, and 23 post tumor inoculation.

As shown in FIG. 4B, thymectomized animals treated with the combination treatment regimen of IL-7 protein and anti-PD-1 antibody had significantly reduced tumor volume compared to other treatment groups (control, IL-7 protein alone, and anti-PD-1 antibody alone).

### Example 5 Effect of IL-7 Protein and PD-1 Pathway Inhibitor Combination Treatment on Tumor-Infiltrating Lymphocytes in Thymectomy-Induced Lymphopenia

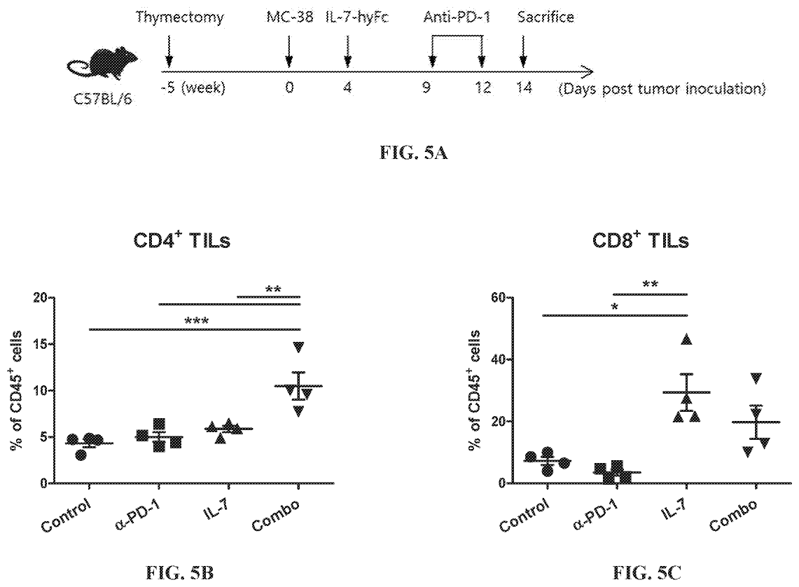

To assess whether IL-7 protein in combination with PD-1 pathway inhibitor has any effect on TILs in a lymphopenic environment, C57BL/6 mice were thymectomized as described in Example 4. At 5 weeks after surgery, MC-38 colon adenocarcinoma tumor cells (1×10⁵, subcutaneously) were transplanted into the animals. On day 4 post tumor inoculation, the animals were treated with a single dose of IL-7 protein (1.25 mpk or 25 μg/mouse, subcutaneously) or IL-7-formulating buffer. See FIG. 5A. Then, on days 9 and 12 post tumor inoculation, animals were treated with anti-PD-1 antibody (5 mpk or 100 μg/mouse, intraperitoneally) or isotype control antibody. Animals were sacrificed on day 14 post tumor inoculation, and the tumor-infiltrating lymphocytes in the different animals were analyzed with flow cytometry.

As observed in the non-lymphopenic animals (see FIG. 2B), treatment of the thymectomized animals with both IL-7 protein and anti-PD-1 antibody resulted in a significant increase in the percentage of CD4⁺ TILs in the tumors compared to the other treatment groups. FIG. 5B. There was also a significant increase in the percentage of CD8⁺ TILs. As shown in FIG. 5C, thymectomized animals treated with both IL-7 protein and anti-PD-1 antibody had significantly higher percentage of CD8⁺ TILs in the tumors compared to both the control group and anti-PD-1 antibody alone group. The increase was comparable to that observed in the IL-7 protein alone treatment group.

Collectively, the above results (i.e., Examples 4 and 5) demonstrate that IL-7 protein and PD-1 pathway inhibitor combination therapy can also effectively treat cancer even under lymphopenic conditions.

### Example 6: Effect of IL-7 Protein on T Cell Proliferation and Activation

To better understand the anti-tumor effects of IL-7 protein disclosed herein, the effect of IL-7 protein on the proliferation and activation of T cells was first assessed in normal mice. Briefly, C57BL/6 mice were subcutaneously treated 10 mg/kg of IL-7 protein. Controls animals received buffer alone. The animals were bled at various time points (i.e., days 2, 4, 5, 6, 8, 10, 12, and 14) post administration and the percentages of different CD8+ T cell populations were assessed using flow cytometry. At day 5 post treatment, some of the animals were sacrificed, and the CD8+ T cells from the spleen were assessed for the expression of different activation markers (T-bet, Eomes, PD-1, Granzyme B (GzmB), CXCR3) and cytokine production (IFN-γ, TNF-α, and IL-2). Cytokine production was assessed using intracellular cytokine staining after ex vivo PMA/ionomycin stimulation.

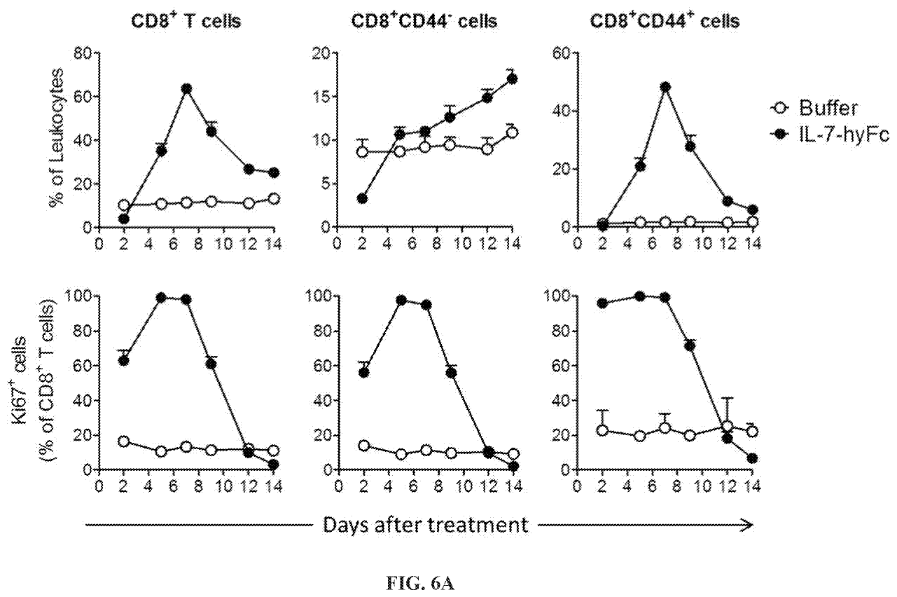

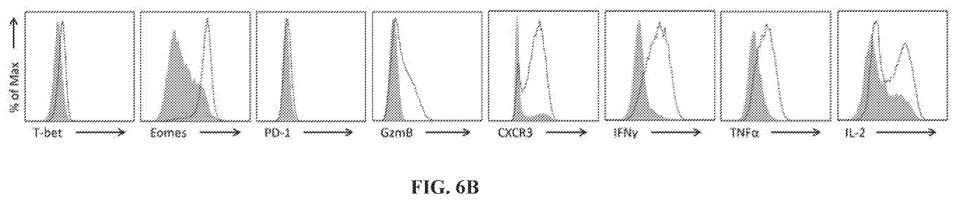

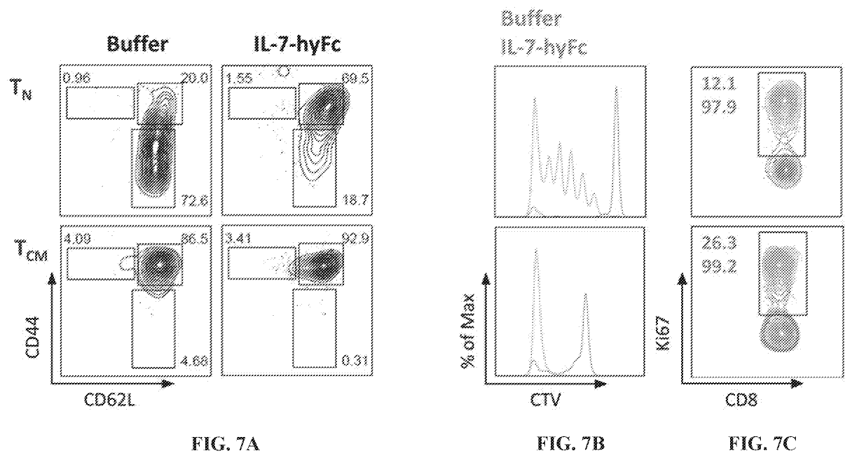

As shown in FIG. 6A, IL-7 protein administration into normal mice resulted in increased CD8+ T cell proliferation (as evidenced by increased Ki-67 expression) compared to the control animals. The greatest effect was observed among the CD44+ (central memory) CD8+ T cell population. In addition to the increased proliferation, the splenic CD8+ T cells from the IL-7 protein treated animals also expressed higher levels of T-bet, Eomes, PD-1, Granzyme B, and CXCR3, suggesting that the cells were more activated compared to those from the control animals (see FIG. 6B). Greater percentage of the cells also produced IFN-γ, TNF-α, and IL-2 after ex vivo stimulation.

Next, to further assess the effect of IL-7 protein on T cells, naïve (10⁶ cells/mouse) or central memory (5×10⁵ cells/mouse) T cells were labeled with CELLTRACE™ Violet (CTV) and adoptively transferred into congenic mice. One day after transfer, the recipient animals were subcutaneously treated with IL-7 protein (10 mg/kg) or buffer. Five days after treatment, the animals were sacrificed, and the splenic CD8+ T cells analyzed.

As shown in FIG. 6C, IL-7 administration increased the proliferation of both naïve and central memory CD8+ T cells. However, the greatest effect was observed among the central memory CD8 T cells, confirming the results observed above with normal C57BL/6 mice. These results demonstrate that that the administration of IL-7 protein disclosed herein can induce the proliferation and activation of T cells.

### Example 7: Analysis of the Anti-Tumor Effects of IL-7 Protein

To better characterize the anti-tumor effects of IL-7 protein disclosed herein, C57BL/6 mice were transplanted with MC-38 colon adenocarcinoma tumor cells (1×10⁵, subcutaneously). At day five post-tumor inoculation, the mice were treated with one of the following concentrations of IL-7 protein: (i) 0 mg/kg (i.e., buffer alone), (ii) 1.25 mg/kg, (iii) 2.5 mg/kg, (iv) 5 mg/kg, and (v) 10 mg/kg. Tumor volume was assessed periodically after administration. At day seven post-treatment, animals were bleed and the percentages of various immune cells (i.e., CD8+ T cells, CD4+ T cells, Foxp3+ CD4+ regulatory T cells, B220+ cells) were assessed.

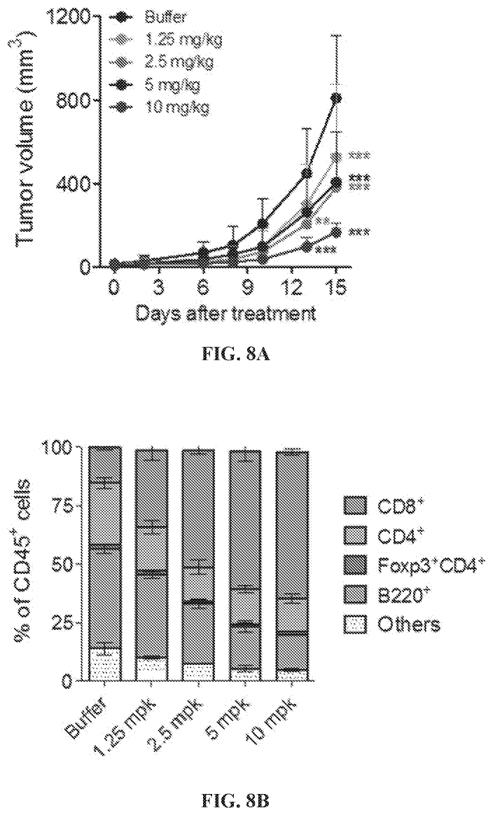

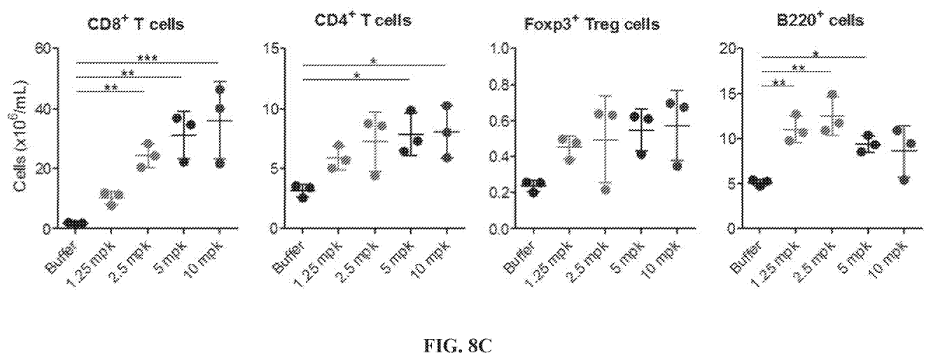

As shown in FIG. 8A, administration of IL-7 protein to the tumor mice resulted in a dose-dependent reduction in tumor volume. The decrease in tumor volume correlated with a significant increase in CD8+ T cell numbers in the peripheral blood (see FIGS. 8B and 8C). These results demonstrate the anti-tumor effects of IL-7 protein administration in tumor mice.

### Example 8: Analysis of the Tumor Microenvironment after IL-7 Protein Administration

To further characterize the anti-tumor effects of IL-7 protein, MC38 colon adenocarcinoma tumor cells were transplanted into C57BL/6 mice as described in the earlier Examples. Then, at day five post-tumor inoculation, the animals were subcutaneously treated with 10 mg/kg of IL-7 protein or buffer. At day seven post-treatment, the animals were sacrificed and the tumor tissues were assessed for the presence of different immune cells (i.e., monocytic myeloid-derived suppressor cells (M-MDSCs), polymorphonuclear myeloid-derived suppressor cells (PMN-MDSCs), tumor associated macrophages (TAMs), tumor associated dendritic cells (TADCs), CD8+ T cells, CD4+ T cells, Foxp3+ CD4+ regulatory T cells (Tregs), NK cells, and B cells).

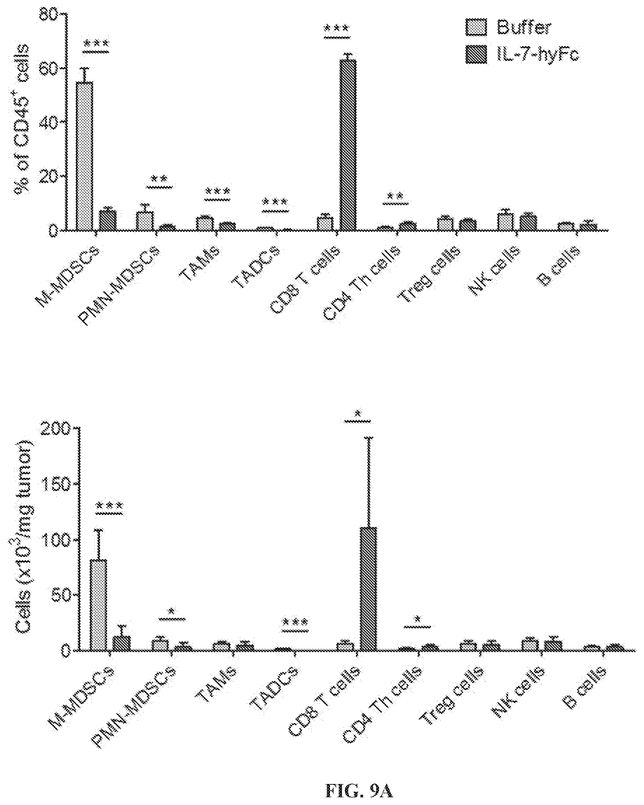

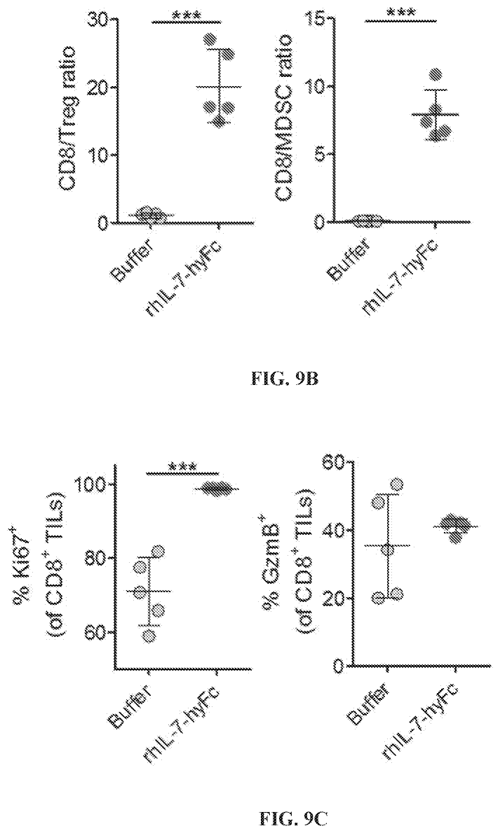

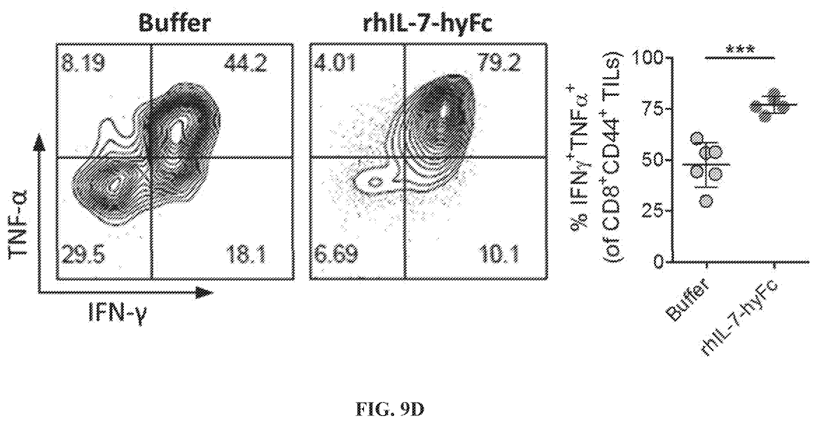

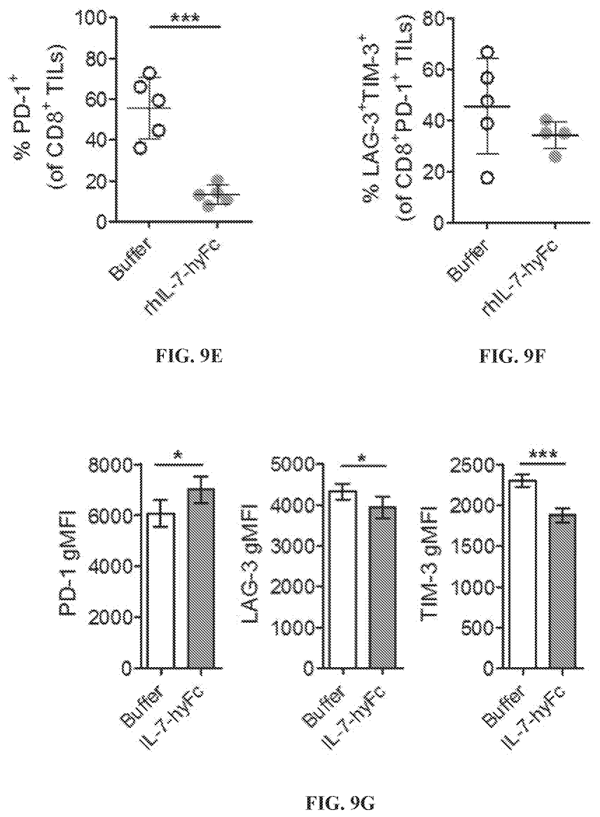

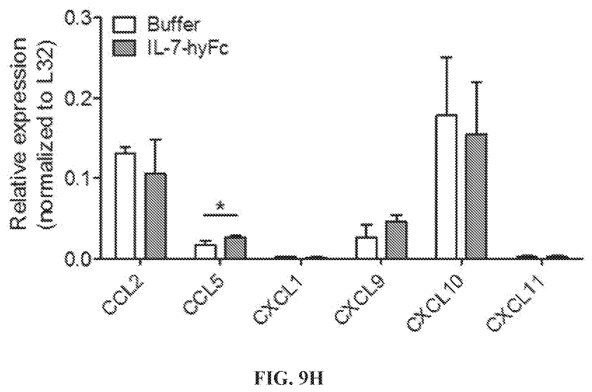

As observed in the peripheral blood (see Example 7), IL-7 protein administration resulted in significant increase in the number of CD8+ T cells in the tumors (TILs), yielding a high CD8+ T/Treg cell ratio in the tumor microenvironment (see FIGS. 9A and 9B). Compared to the cells from the control animals, greater percentage of the TILs of mice treated with IL-7 protein expressed Ki-67 and granzyme B, indicating that they were more highly activated (see FIG. 9C). The TILs from the IL-7 treated animals were also more potent producers of IFN-γ and TNF-α, and expressed lower levels of inhibitor receptors, such as PD-1 and LAG-3 (see FIGS. 9D-9G). Interestingly, IL-7 protein administration reduced the number of myeloid-derived suppressor cells (MDSCs) in the tumor microenvironment, resulting in increased CD8+ T/MDSC ratio (see FIGS. 9A and 9B). Other than a moderate increase in CCL5 expression, there was no significant difference in chemokine expression within the tumor lysates from mice treated with IL-7 protein and those treated with buffer alone (FIG. 9H).

These results demonstrate that IL-7 protein disclosed herein can confer anti-cancer activity by inducing a CD8+ T cell infiltrated-inflamed-immune favorable tumor microenvironment.

### Example 9: Analysis of the Anti-Tumor Effects of IL-7 Protein in Combination with Other Anti-Cancer Agents

To further assess whether the anti-tumor effects of IL-7 protein can be enhanced when combined with other anti-cancer agents, C57BL/6 mice were inoculated with MC38 colon adenocarcinoma tumor cells as described in the earlier Examples. Then, the mice were treated with one of the following: (i) buffer alone, (ii) cyclophosphamide (CPA) (100 mg/kg) in combination with an immune checkpoint inhibitor (10 mg/kg), and (iii) triple combination of CPA, immune checkpoint inhibitor, and IL-7 protein (10 mg/kg). The immune checkpoint inhibitor used included: anti-PD-1 antibody, anti-PD-L1 antibody, or anti-CTLA-4 antibody. CPA was administered to the animals intraperitoneally on day 10 after tumor inoculation. The immune checkpoint inhibitors were administered intraperitoneally from day six post CPA treatment (every 3 days for a total of 5 doses). IL-7 protein was administered subcutaneously on day 2 after CPA treatment. Both tumor volume and survival were assessed at various time points post-treatment.

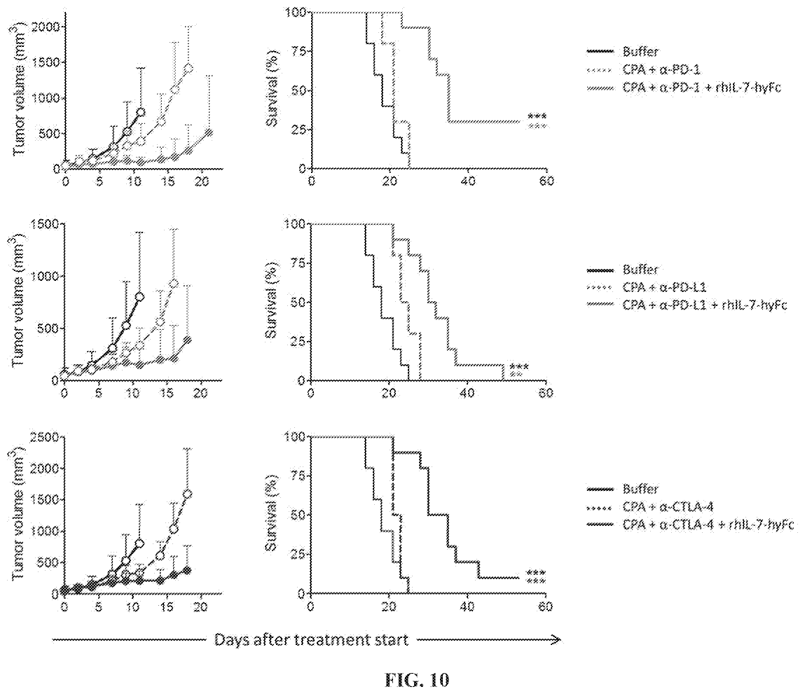

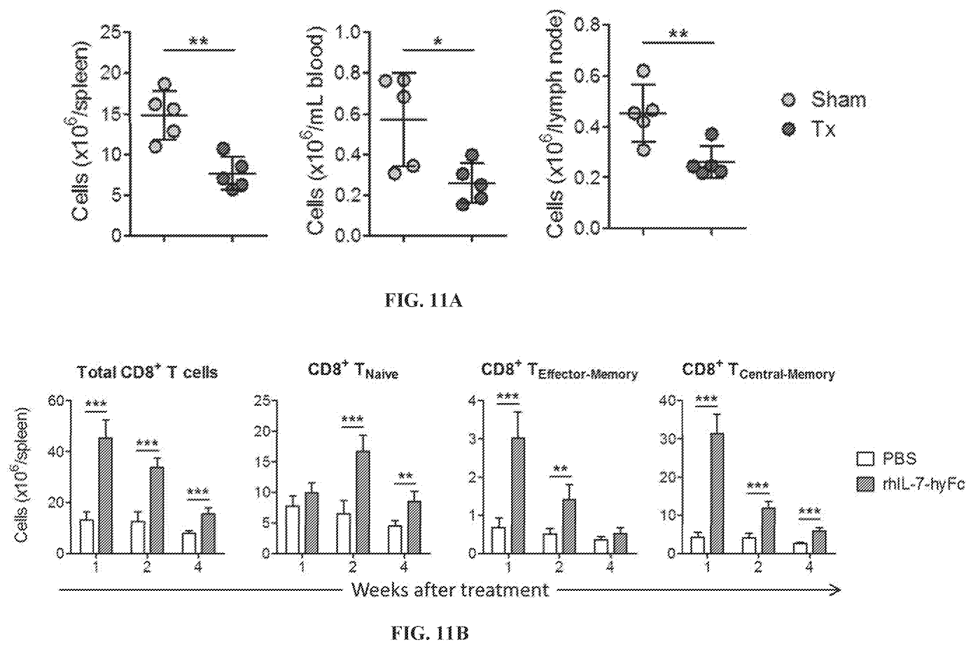

As shown in FIG. 10, animals treated with the triple combination (i.e., CPA+immune checkpoint inhibitor+IL-7 protein) had the greatest tumor reduction. The increased tumor reduction correlated with increased survival. These results demonstrate that IL-7 can significantly enhance the anti-tumor efficacy of other immunochemotherapy treatments.

### Example 10: Analysis of the Anti-Tumor Effects of IL-7 Protein in Thymectomy-Induced Lymphopenia

To better characterize the anti-tumor effects of IL-7 protein in a lymphopenic environment, C57BL/6 mice were thymectomized as described in Example 4. As shown in FIG. 11A, compared to the sham control (i.e., same surgical procedure except the thymus was not removed), the thymectomized animals expressed lower numbers of CD8+ T cells in the spleen, blood, and lymph nodes, confirming their lymphopenic condition. Upon recovery from surgery, the animals were treated with PBS or IL-7 protein (1.25 mg/kg, subcutaneously). Then, the animals were sacrificed at weeks 1, 2, and 4 post-treatment, and the number of different CD8+ T cell populations in the spleen was assessed.

Similar to that observed in normal C57BL/6 mice (see Example 6), 11B, IL-7 protein administration to the thymectomized animals resulted in greater number of CD8+ T cells compared to the sham control (FIG. 11B). The increase in number was observed for all CD8+ T cell populations analyzed, i.e., naïve (CD44− CD62L+), effector-memory (CD44+ CD62L-), and central memory (CD44+ CD62L+).

### Example 11: Analysis of the Anti-Tumor Effects of IL-7 Protein in Advanced Solid Cancer Patients

A phase 1b clinical trial was conducted to evaluate the safety and efficacy of the IL-7 protein disclosed herein (i.e., comprising hyFc; “IL-7-hyFc”) in advanced solid cancer patients. The primary objectives of the study were (i) to assess the safety and tolerability of IL-7-hyFc; and (ii) to determine the maximum tolerable dose (MTD), recommended phase 2 dose (RP2D), and the dose-limiting toxicity (DLT) in the patients. The secondary objectives included determining the (i) pharmacokinetics and pharmacodynamics, and (ii) immunogenicity of IL-7-hyFc in the patients. Exploratory biomarkers were also assessed.

To be eligible for the study, the patients had to meet the following criteria: (i) ≥19 years old; (ii) Eastern Cooperative Oncology Group (ECOG) performance status score of 0-1; (iii) life expectancy of ≥12 weeks; (iv) measurable disease per RECIST v1.1; and (v) locally advanced or metastatic solid tumor. In total, 21 patients were enrolled in the study (10 colon cancer, 5 rectal cancer, 2 breast cancer, 1 ovary cancer, 1 synovial sarcoma, 1 anal cancer, and 1 cervical cancer).

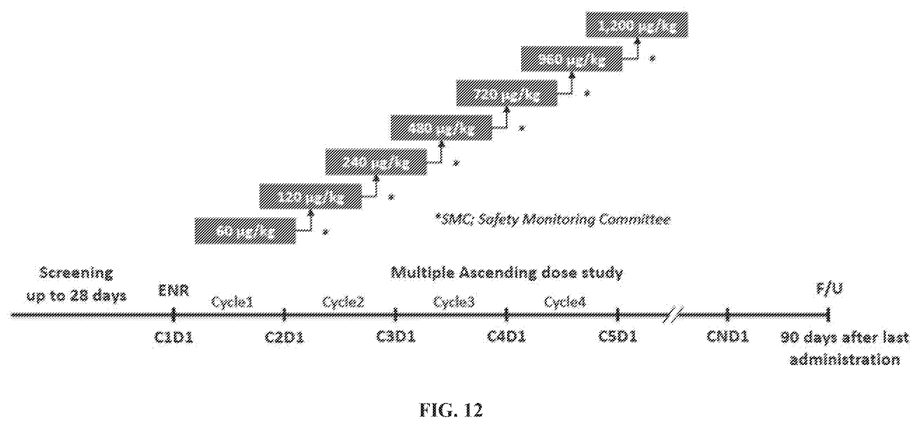

A traditional 3+3 dose escalation design was implemented using the following dose increments: (i) 60 μg/kg, (ii) 120 μg/kg, (iii) 240 μg/kg, (iv) 480 μg/kg, (v) 720 μg/kg, (vi) 960 μg/kg, and (vii) 1,200 μg/kg. IL-7-hyFc was administered intramuscularly with the patients receiving IL-7-hyFc every three weeks. FIG. 12 provides a schematic of the overall study design.

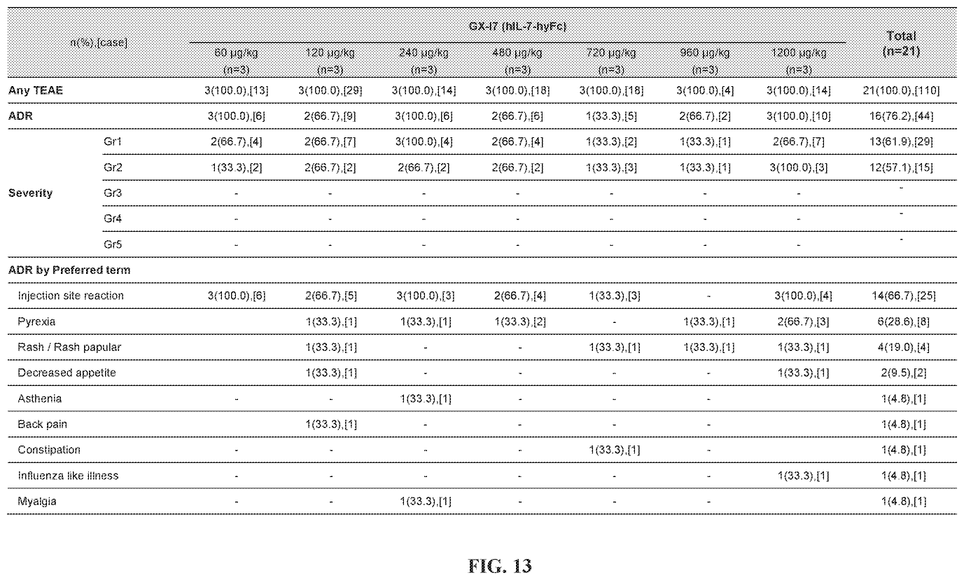

At various time points throughout the trial, patients were screened for any adverse events using one or more of the following: abnormal laboratory tests, clinical symptoms and signs described by the subjects, and investigator evaluation. As shown in FIG. 13, there were 44 cases of adverse drug event (ADR) for all doses tested, with majority of them involving injection site reactions, which were manageable with conventional use of anti-histamines and/or corticosteroids.

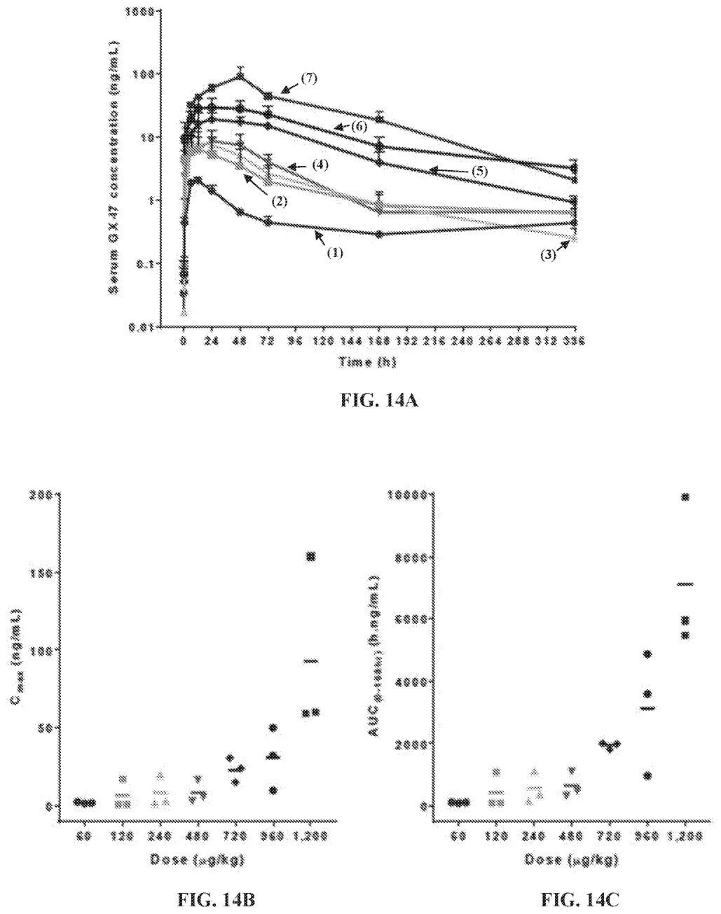

To characterize the pharmacokinetic profile of IL-7-hyFc in the advanced solid cancer patients, blood was collected prior to IL-7-hyFc administration and then at 0.5, 6, 12, 24, 48, 72, 168, 336, and 504 hours after administration. Concentration of IL-7 was determined using ELISA (Human IL-7 Quantikine HS ELISA Kit HS750; R&D Systems). As shown in FIG. 14A, concentration of IL-7-hyFc peaked on average between 12 and 48 hours after administration with a half-life of about 33 to 147 hours. There was a dose dependent increase in C. and AUC (see FIGS. 14B and 14C).

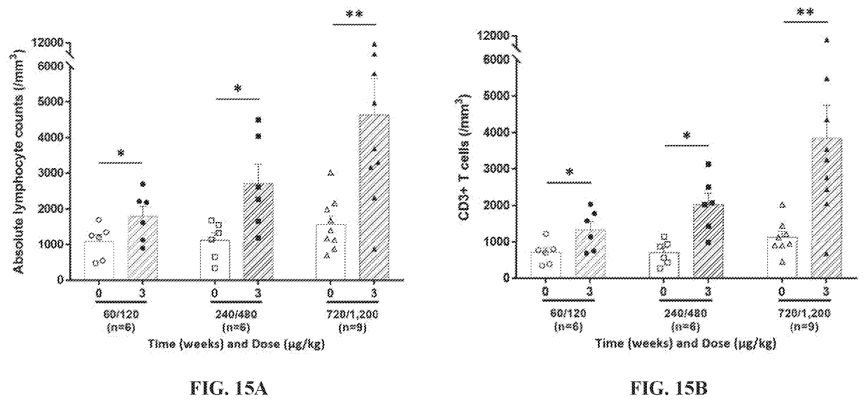

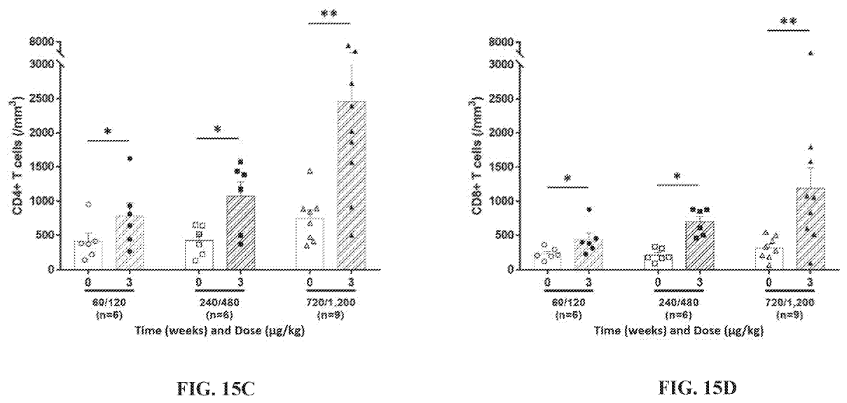

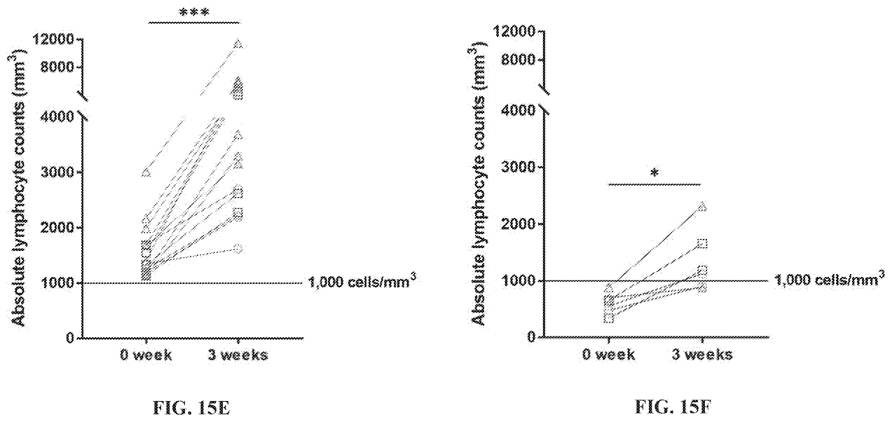

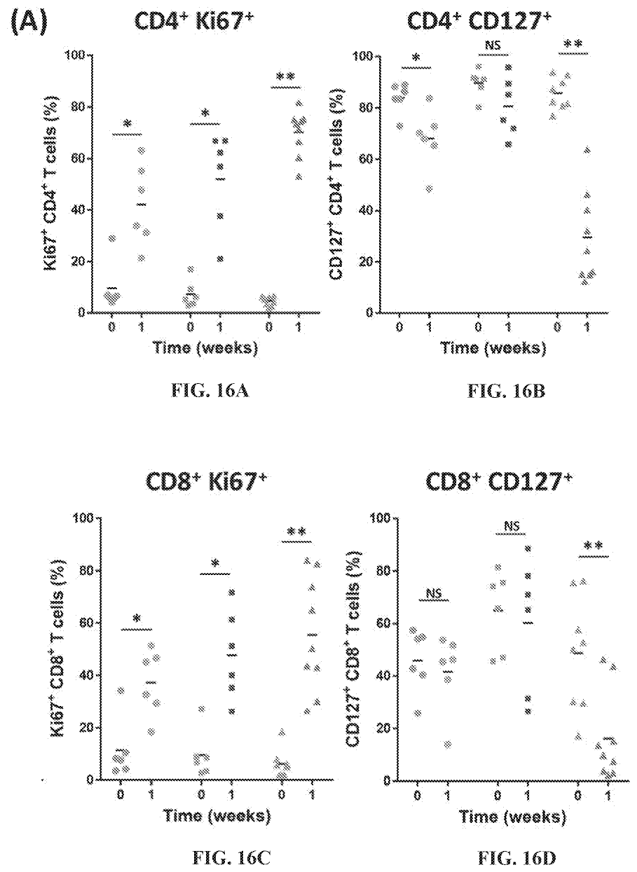

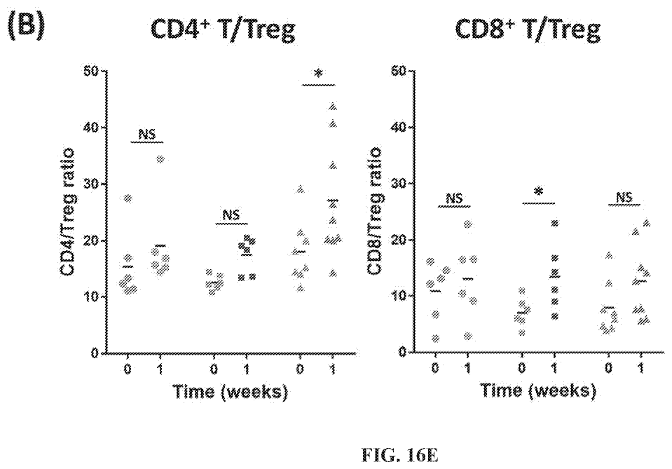

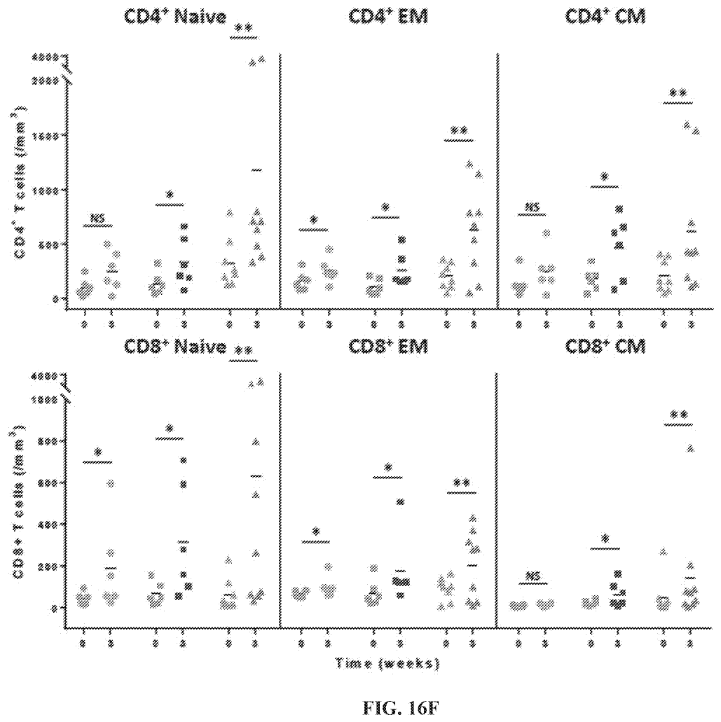

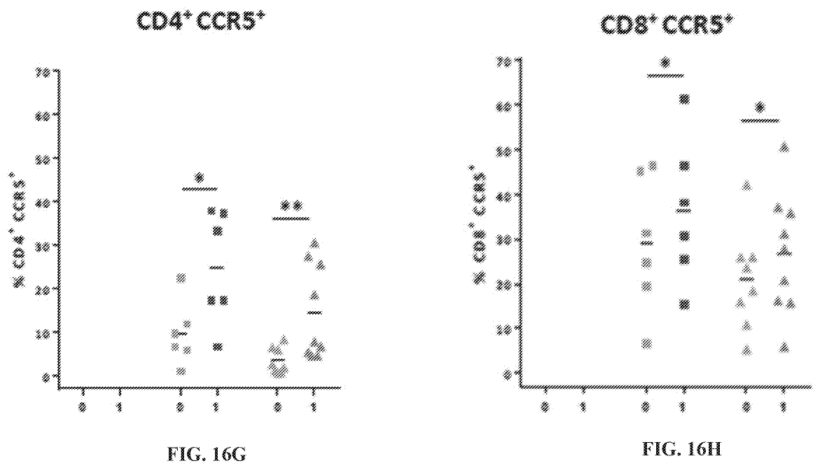

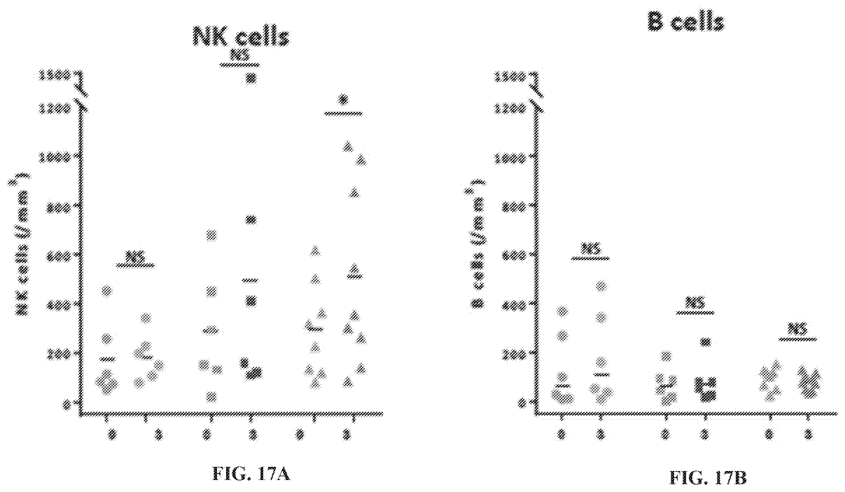

To characterize the pharmacodynamics profile of IL-7-hyFc, blood was collected from the advanced solid cancer patients prior to administration and then at three weeks post administration. Then, various biological markers were used to calculate the absolute lymphocyte count (ALC) as well as different lymphocyte subsets. As shown in FIGS. 15A to 15D, there was also a dose-dependent increase in ALC, CD3+, CD4+ and CD8+ T cells in the subjects. The effect on ALC was observed in both lymphopenic (ALC<1,000 cells/mm³) and non-lymphopenic (ALC≥1,000 cells/mm³) patients (see FIGS. 15E and 15F). Patients that received IL-7-hyFc also exhibited greater CCR5 expression on both CD4+ and CD8+ T cells (see FIGS. 16G and 16H). Similarly, patients that received the higher doses (720-1,200 μg/kg) had greater percentage of Ki67+ CD4+ and CD8+ T cells (see FIGS. 16A and 16C), compared to patients that received one of the lower doses. At the higher doses (720-1,200 μg/kg), there was a noticeable decrease in IL-7Rα expression on both the CD4+ and CD8+ T cells (see FIGS. 16B and 16D). IL-7-hyFc appeared to have less of an effect on regulatory T cells (Tregs), as advanced solid cancer patients that received IL-7-hyFc appeared to have greater CD4+/Treg or CD8+/Treg ratio at certain doses (see FIG. 16E). The dose dependent effect of IL-7-hyFc on the T cells was observed among all naïve, effector memory (EM), and central memory (CM) CD4+ and CD8+ T cells (see FIG. 16F). While an increase in NK cells was observed at high doses (720-1,200 μg/kg), administration of IL-7-hyFc to patients with advanced solid cancer did not have an effect on B cells, for all doses tested (see FIGS. 17A and 17B).

The above data demonstrate that IL7-hyFc is generally safe and can be efficacious for treating advanced solid cancers, even at the higher doses (e.g., 720-1,200 μg/kg) with a dosing interval of once every three weeks.

### Example 12: Analysis of the Anti-Tumor Effects of IL-7 Protein in Patients with Glioblastoma

The above clinical trial (see Example 11) also evaluated the safety and efficacy of IL7-hyFc in treating glioblastoma. The overall study design was the same as in Example 11 (e.g., 3+3 traditional dose escalation and similar eligibility requirements), but the doses for the glioblastoma trial were as follows: (i) 60 μg/kg, (ii) 360 μg/kg, (iii) 600 μg/kg, (iv) 840 μg/kg, and (v) 1,440 μg/kg. In total, 15 patients were enrolled in the study.

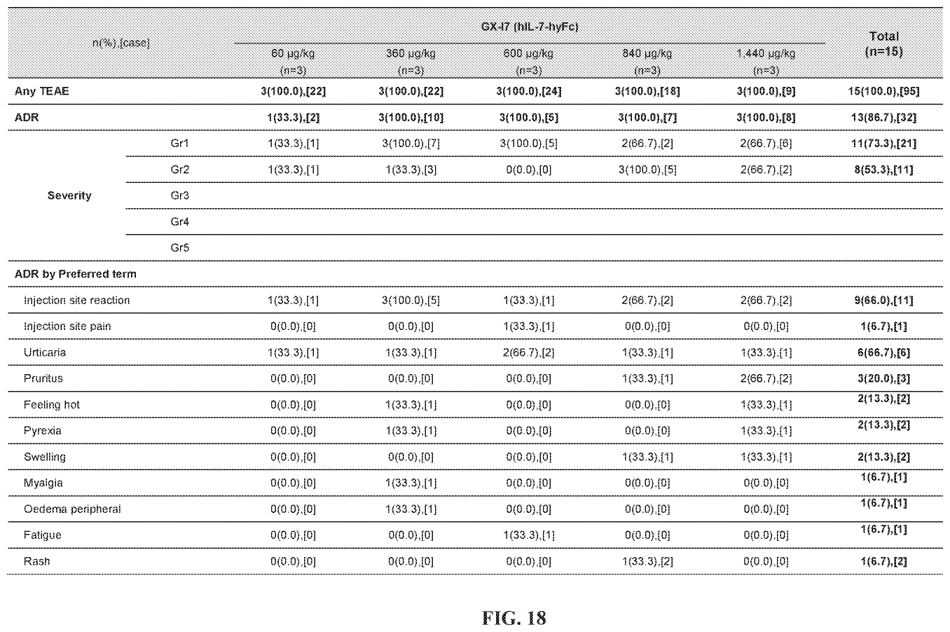

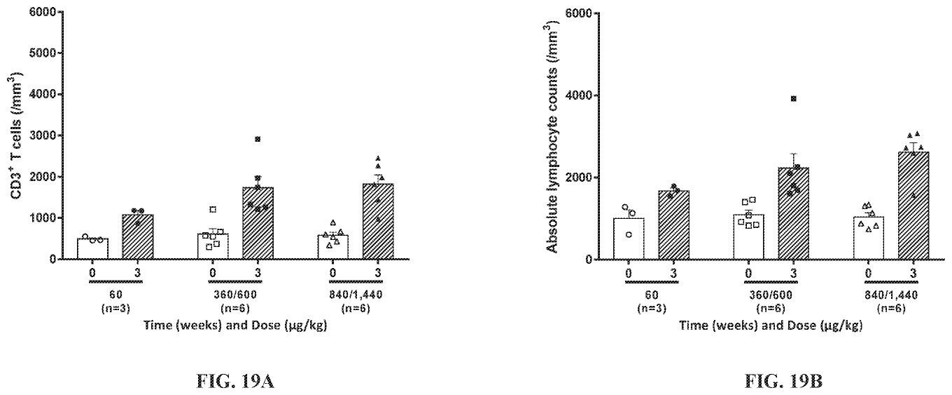

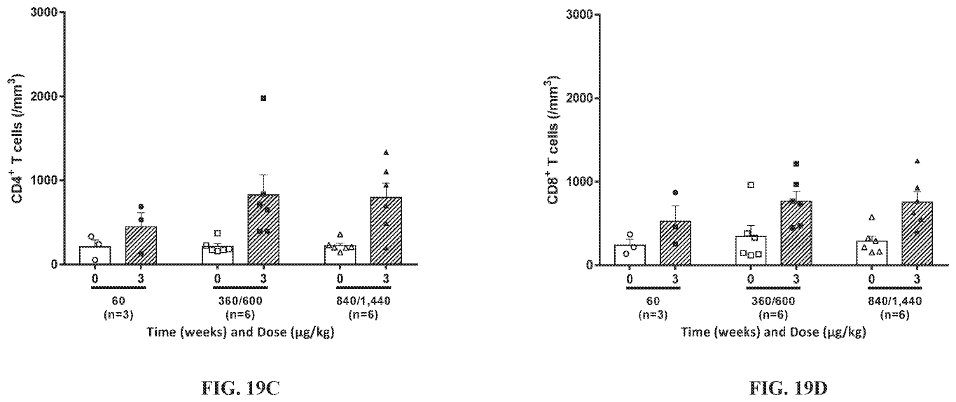

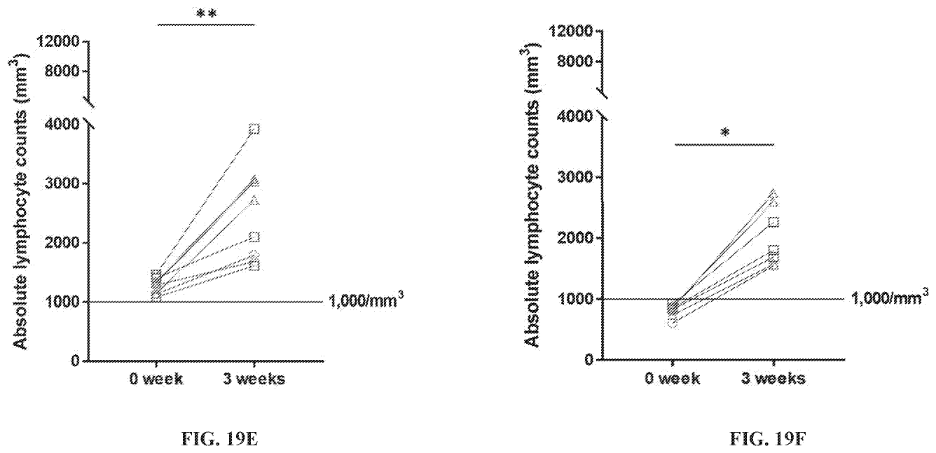

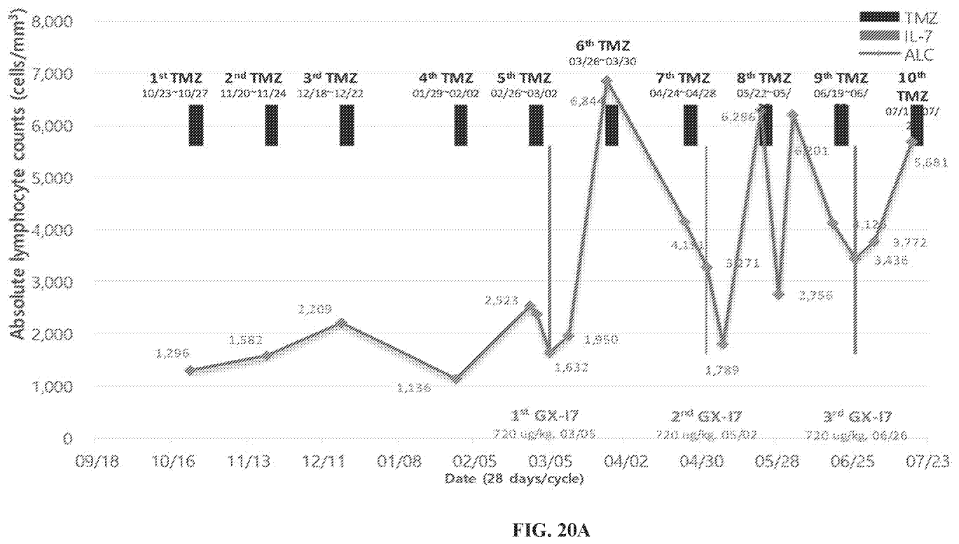

As observed with the advanced solid cancer patients, administration of IL7-hyFc to the glioblastoma patients was generally well-tolerated for all doses tested (see FIG. 18). Again, the most common adverse drug event was injection site reaction, which was readily treatable. The overall pharmacodynamics and pharmacokinetic profiles were also similar as that observed in the advanced solid cancer patients. Administration of IL7-hyFc to the glioblastoma patients also resulted in a dose-dependent increase in ALC, CD3+, CD4+ and CD8+ T cells in both lymphopenic and non-lymphopenic patients (see FIGS. 19B to 19F).

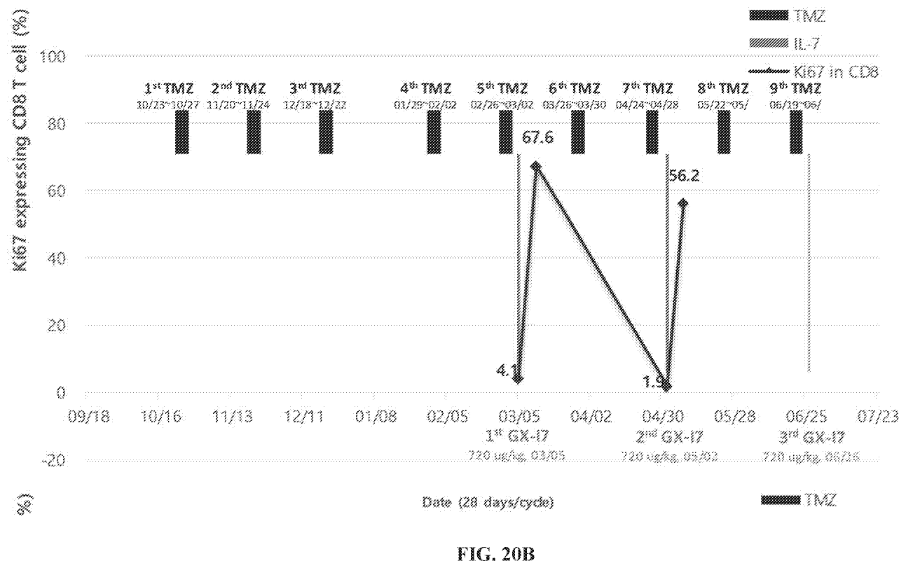

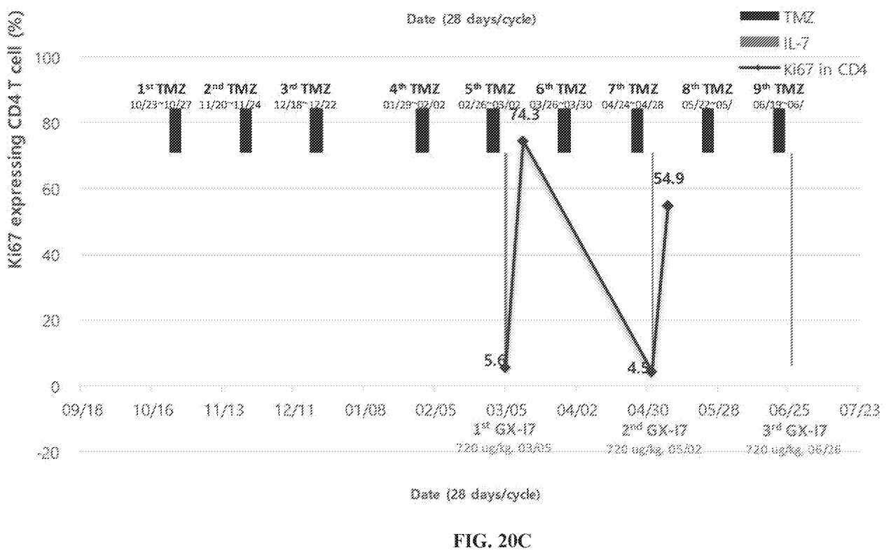

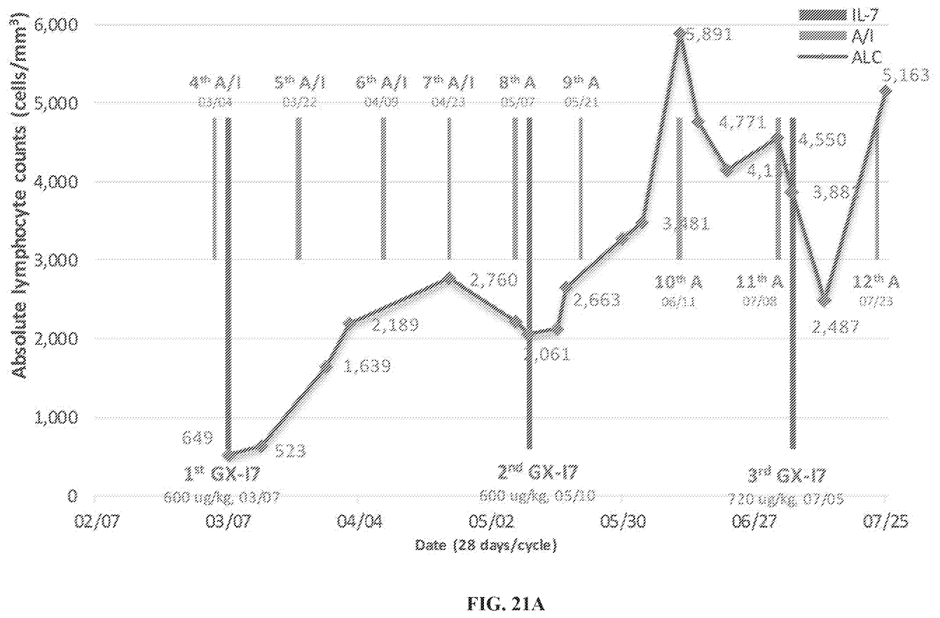

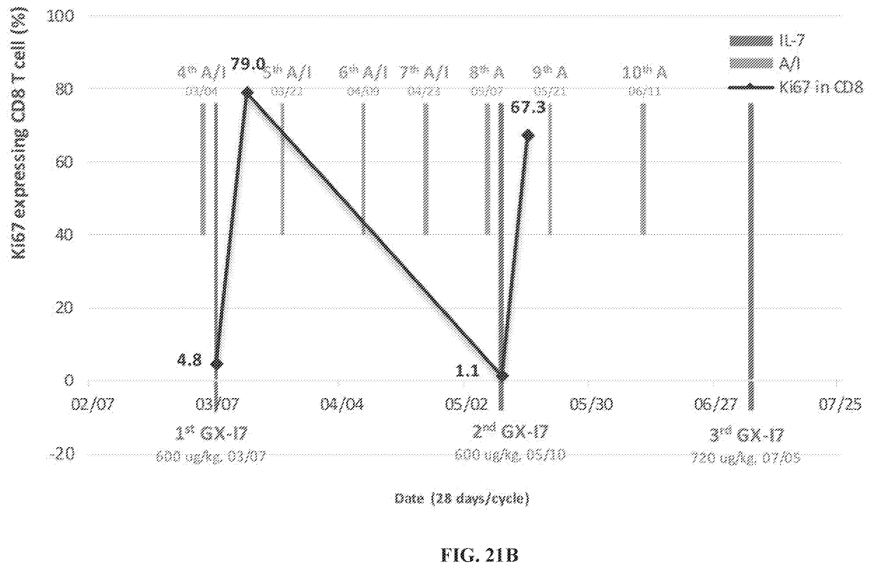

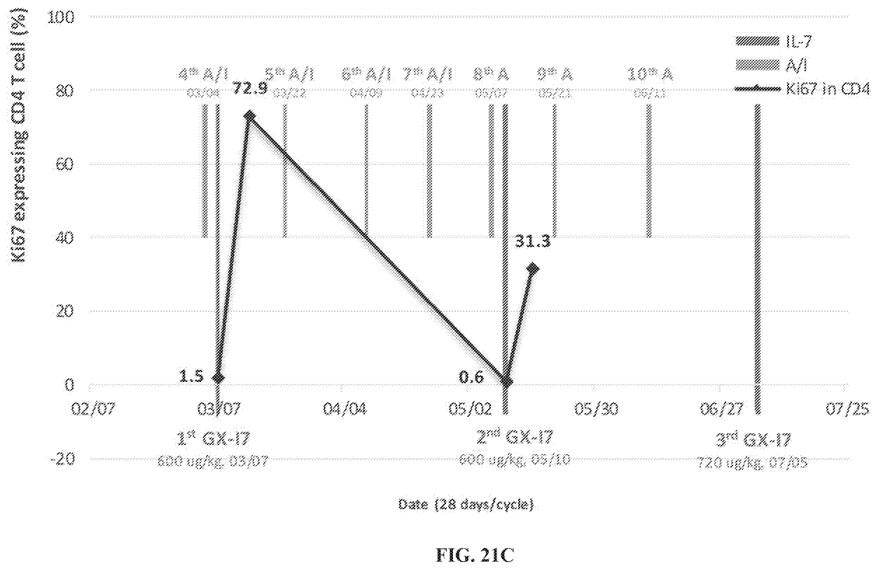

Among the glioblastoma patients, the effect of IL7-hyFc administration on the effect of different chemotherapeutic agents was also assessed. As shown in FIGS. 20A to 20C, IL7-hyFc administration (dose of 720 μg/kg at a dosing interval of once in every eight weeks) also increased ALC and the frequency of Ki67+ CD4+ and CD8+ T cells in glioblastoma subjects that received temozolomide. Similar results were observed in glioblastoma patients that received avastin/irinotecan when IL7-hyFc was administered to the subjects at doses of 600-720 μg/kg at a dosing interval of once in every 12 weeks (see FIGS. 21A to 21C).

Collectively, the above data show that IL7-hyFc is safe and can have potential therapeutic effects on both advanced solid cancers and glioblastoma. The therapeutic effects observed above (e.g., the ability to increase T cells in different cancer types) suggests that IL7-hyFc can be effective in treating various cancers, particularly in combination with other anti-cancer treatment regimens, such as an immune checkpoint inhibitor.

What is claimed is:

1. A method of treating a tumor in a human subject in need thereof, comprising administering to the subject an effective amount of an interleukin-7 (IL-7) protein in combination with an effective amount of a Programmed Death-1 (PD-1) pathway inhibitor, wherein a tumor volume is decreased in the subject after the administration compared to a reference tumor volume after administration of either the PD-1 pathway inhibitor alone or IL-7 protein alone.

2. The method of claim 1, wherein the tumor volume is decreased by at least about 5%, at least about 10%, at least about 20%, at least about 30%, at least about 40%, at least about 50%, at least about 60%, at least about 70%, at least about 80%, at least about 90%, or at least about 100% after the administration.

3. The method of claim 1 or 2, wherein a number of tumor infiltrating lymphocytes (TILs) in the tumor is increased after the administration compared to a number of TILs in a tumor after administration of either the PD-1 pathway inhibitor alone or IL-7 protein alone.

4. The method of claim 3, wherein the TILs are CD4⁺ TILs.

5. The method of claim 3, wherein the TILs are CD8⁺ TILs.

6. The method of any one of claims 3 to 5, wherein the number of TILs is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration.

7. The method of any one of claims 1 to 6, wherein the human subject exhibits a lymphopenia prior to the administration.

8. A method of treating a tumor in a subject in need thereof, comprising administering to the subject an effective amount of an interleukin-7 (IL-7) protein in combination with an effective amount of a Programmed Death-1 (PD-1) pathway inhibitor, wherein the subject exhibits a lymphopenia.

9. The method of claim 7 or 8, wherein the human subject exhibiting lymphopenia has T lymphopenia, B lymphopenia, and/or NK lymphopenia.

10. The method of any one of claims 7 to 9, wherein the lymphopenia is caused by or associated with the tumor.

11. The method of any one of claims 7 to 10, wherein the lymphopenia is caused by or associated with a previous therapy for the tumor.

12. The method of any one of claims 7 to 11, wherein the lymphopenia is caused by an infection, chronic failure of the right ventricle of the heart, Hodgkin's disease and cancers of the lymphatic system, leukemia, a leak or rupture in the thoracic duct, side effects of prescription medications including anticancer agents (e.g., chemotherapy), antiviral agents, and glucocorticoids, malnutrition resulting from diets that are low in protein, radiation therapy, uremia, autoimmune disorders, immune deficiency syndromes, high stress levels, trauma, thymectomy, or a combination thereof.

13. The method of any one of claims 7 to 12, wherein the lymphopenia is idiopathic.

14. The method of any one of claims 7 to 13, wherein the lymphopenia comprises an idiopathic CD4 positive T-lymphocytopenia (ICL), acute radiation syndrome (ARS), or a combination thereof.

15. The method of any one of claims 7 to 14, wherein the lymphopenia is characterized by a circulating blood total lymphocyte count that is less than by at least about 5%, at least about 10%, at least about 20%, at least about 30%, at least about 40%, at least about 50%, at least about 60%, at least about 70%, at least about 80%, at least about 90%, or at least about 100% compared to a circulating blood total lymphocyte count in a corresponding subject who does not exhibit a lymphopenia.

16. The method of any one of claims 7 to 15, wherein the lymphopenia is characterized by a circulating blood total lymphocyte count of less than about 1,500 lymphocytes/μL, less than about 1,000 lymphocytes/μL, less than about 800 lymphocytes/μL, less than about 500 lymphocytes/μL, or less than about 200 lymphocytes/μL.

17. The method of any one of claims 8 to 16, wherein a number of tumor infiltrating lymphocytes (TILs) in the tumor is increased after the administration compared to a number of TILs in a tumor after administration of either the PD-1 pathway inhibitor alone or IL-7 protein alone.

18. The method of claim 17, wherein the number of TILs is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration.

19. The method of claim 17 or 18, wherein the TILs are CD4⁺ TILs.

20. The method of claim 17 or 18, wherein the TILs are CD8⁺ TILs.

21. The method of any one of claims 1 to 20, wherein the IL-7 protein is not a wild type IL-7.

22. The method of any one of claims 1 to 21, wherein the IL-7 protein comprises an oligopeptide consisting of 1 to 10 amino acid residues.

23. The method of claim 22, wherein the oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine.

24. The method of claim 23, wherein the oligopeptide is methionine-glycine-methionine.

25. The method of any one of claims 1 to 24, wherein the IL-7 protein comprises a half-life extending moiety.

26. The method of claim 25, wherein the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

27. The method of claim 26, wherein the half-life extending moiety is an Fc.

28. The method of claim 27, wherein the Fc is a hybrid Fc, comprising a hinge region, a CH2 domain, and a CH3 domain,
wherein the hinge region comprises a human IgD hinge region,
wherein the CH2 domain comprises a part of human IgD CH2 domain and a part of human IgG4 CH2 domain, and
wherein the CH3 domain comprises a part of human IgG4 CH3 domain.

29. The method of any one of claims 1 to 28, wherein the IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6 and 15-25.

30. The method of any one of claims 1 to 29, wherein the PD-1 pathway inhibitor comprises an anti-PD-1 antibody or an anti-PD-L1 antibody.

31. The method of claim 30, wherein the anti-PD-1 antibody comprises nivolumab, pembrolizumab, MEDI0608, AMP-224, PDR001, BGB-A317, or any combination thereof.

32. The method of claim 31, wherein the anti-PD-L1 antibody comprises BMS-936559, MPDL3280A, MEDI4736, MSB0010718C, or any combination thereof.

33. The method of any one of claims 1 to 32, wherein the IL-7 protein and the PD-1 pathway inhibitor are administered concurrently.

34. The method of any one of claims 1 to 32, wherein the IL-7 protein and the PD-1 pathway inhibitor are administered sequentially.

35. The method of claim 34, wherein the IL-7 protein is administered to the subject prior to administering the PD-1 pathway inhibitor.

36. The method of any one of claims 1 to 35, wherein the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

37. The method of claim 36, wherein the breast cancer is a triple negative breast cancer (TNBC).

38. The method of claim 36, wherein the brain cancer is a glioblastoma.

39. The method of claim 36, wherein the skin cancer is a basal cell carcinoma (BCC), cutaneous squamous cell carcinoma (cSCC), melanoma, Merkel cell carcinoma (MCC), or a combination thereof.

40. The method of claim 36, wherein the head and neck cancer is a head and neck squamous cell carcinoma.

41. The method of claim 36, wherein the lung cancer is a small cell lung cancer (SCLC).

42. The method of claim 36, wherein the esophageal cancer is gastroesophageal junction cancer.

43. The method of claim 36, wherein the kidney cancer is renal cell carcinoma.

44. The method of claim 36, wherein the liver cancer is hepatocellular carcinoma.

45. The method of any one of claims 1 to 44, wherein the IL-7 protein is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

46. The method of any one of claims 1 to 45, wherein the PD-1 pathway inhibitor is administered to the subject parenthetically, intramuscularly, subcutaneously, intravenously, or intraperitoneally.

47. A method of treating a tumor in a human subject in need thereof, comprising administering to the subject an effective amount of an interleukin-7 (IL-7) protein in combination with an effective amount of a CTLA-4 pathway inhibitor.

48. The method of claim 47, wherein a tumor volume is decreased by at least about 5%, at least about 10%, at least about 20%, at least about 30%, at least about 40%, at least about 50%, at least about 60%, at least about 70%, at least about 80%, at least about 90%, or at least about 100% after the administration.

49. The method of claim 47 or 48, wherein the human subject exhibits a lymphopenia prior to the administration.

50. The method of any one of claims 47 to 49, wherein the CTLA-4 pathway inhibitor comprises an anti-CTLA-4 antibody.

51. The method of claim 50, wherein the anti-CTLA-4 antibody comprises ipilimumab, tremelimumab (ticilimumab; CP-675,206), AGEN-1884, or combinations thereof.

52. The method of any one of claims 47 to 51, wherein the IL-7 protein and the CTLA-4 pathway inhibitor are administered concurrently.

53. The method of any one of claims 47 to 51, wherein the IL-7 protein and the CTLA-4 pathway inhibitor are administered sequentially.

54. The method of claim 53, wherein the IL-7 protein is administered to the subject prior to administering the CTLA-4 pathway inhibitor.

55. The method of any one of claims 47 to 54, wherein the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

56. The method of any one of claims 1 to 55, wherein the IL-7 protein is administered at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

57. The method of any one of claims 1 to 56, wherein the IL-7 protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

58. The method of any one of claims 1 to 57, wherein the IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

59. The method of any one of claims 1 to 58, wherein the IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

60. The method of any one of claims 1 to 59, wherein the IL-7 protein is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks.

61. The method of any one of claims 1 to 60, wherein the IL-7 protein is administered parenthetically.

62. The method of any one of claims 1 to 60, wherein the IL-7 protein is administered intravenously.

63. The method of any one of claims 1 to 62, wherein the IL-7 protein, the PD-1 pathway inhibitor, and/or the CTLA-4 pathway inhibitor are formulated in a composition comprising a bulking agent, stabilizing agent, surfactant, buffering agent, or combinations thereof.

64. The method of claim 63, wherein the PD-1 pathway inhibitor is nivolumab and the composition comprises (a) a mannitol (e.g., about 30 mg), (b) pentetic acid (e.g., about 0.008 mg), (c) polysorbate 80 (e.g., about 0.2 mg), (d) sodium chloride (e.g., about 2.92 mg), and (e) sodium citrate dehydrate (e.g., about 5.88 mg).

65. The method of claim 64, wherein the PD-1 pathway inhibitor is administered to the subject at a flat dose of about 240 mg every two weeks or about 480 mg every four weeks.

66. The method of claim 64, wherein the PD-1 pathway inhibitor is administered to the subject at a weight-based dose of about 3 mg/kg every two weeks.

67. The method of claim 63, wherein the PD-1 pathway inhibitor is pembrolizumab and the composition comprises (a) a L-histidine (e.g., about 1.55 mg), (b) polysorbate 80 (e.g., about 0.2 mg), and (c) sucrose (e.g., about 70 mg).

68. The method of claim 67, wherein the PD-1 pathway inhibitor is administered to the subject at a flat dose of about 200 mg every three weeks.

69. The method of claim 67, wherein the PD-1 pathway inhibitor is administered to the subject at a weight-based dose of about 2 mg/kg every three weeks.

70. The method of claim 63, wherein the PD-1 pathway inhibitor is atezolizumab and the composition comprises (a) a glacial acetic acid (e.g., about 16.5 mg), (b) L-histidine (e.g., about 62 mg), (c) sucrose (e.g., about 821.6 mg), and (d) polysorbate 20 (e.g., about 8 mg).

71. The method of claim 70, wherein the PD-1 pathway inhibitor is administered to the subject at a flat dose of about 1200 mg every three weeks.

72. The method of claim 63, wherein the PD-1 pathway inhibitor is durvalumab and the composition comprises (a) a L-histidine (e.g., about 2 mg), (b) L-histidine hydrochloride monohydrate (e.g., about 2.7 mg), (c) a,a-trehalose dihydrate (e.g., about 104 mg), and (d) polysorbate 80 (e.g., about 0.2 mg).

73. The method of claim 72, wherein the PD-1 pathway inhibitor is administered to the subject at a weight-based dose of about 10 mg/kg every two weeks.

74. The method of claim 63, wherein the PD-1 pathway inhibitor is avelumab and the composition comprises (a) D-mannitol (e.g., about 51 mg), (b) glacial acetic acid (e.g., about 0.6 mg), (c) polysorbate 20 (e.g., about 0.5 mg), and (d) sodium hydroxide (e.g., about 0.3 mg).

75. The method of claim 74, wherein the PD-1 pathway inhibitor is administered to the subject at a flat dose of about 800 mg every two weeks.

76. The method of claim 63, wherein the CTLA-4 pathway inhibitor is ipilimumab and the composition comprises (a) diethylene triamine pentaacetic acid (DTPA) (e.g., about 0.04 mg), (b) mannitol (e.g., about 10 mg), (c) polysorbate 80 (vegetable origin) (e.g., about 0.1 mg), (d) sodium chloride (e.g., about 5.85 mg), and (e) tris hydrochloride (e.g., about 3.15 mg).

77. The method of claim 76, wherein the CTLA-4 pathway inhibitor is administered to the subject at a weight-based dose of about 3 mg/kg every three weeks.

78. The method of claim 76, wherein the CTLA-4 pathway inhibitor is administered to the subject at a weight-based dose of about 10 mg/kg every three weeks for four doses, followed by 10 mg/kg every twelve weeks.

79. The method of any one of claims 63 to 78, wherein the IL-7 protein is formulated in a composition comprising (a) sodium citrate (e.g., about 20 mM), (b) sucrose (e.g., about 5%), (c) sorbitol (e.g., about 1.5%), and (d) Tween 80 (e.g., about 0.05%).


SEQUENCE LISTING (40 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus Musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Cercopithecus aethiops
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, Artificial Sequence
  amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, Artificial Sequence
  amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, Artificial Sequence
  amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, Artificial Sequence
  amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, Artificial Sequence
  amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, Artificial Sequence
  amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, Artificial Sequence
  amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, Artificial Sequence
  amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, Artificial Sequence
  amino acid sequence of modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, Artificial Sequence
  amino acid sequence of modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, Artificial Sequence
  amino acid sequence of modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, Artificial Sequence
  amino acid sequence of modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, Artificial Sequence
  amino acid sequence of modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, Artificial Sequence
  amino acid sequence of modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, Artificial Sequence
  amino acid sequence of modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, Artificial Sequence
  amino acid sequence of modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, Artificial Sequence
  amino acid sequence of modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, Artificial Sequence
  amino acid sequence of modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, Artificial Sequence
  amino acid sequence of modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, Artificial Sequence
  amino acid sequence of human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, Artificial Sequence
  amino acid sequence of human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, Artificial Sequence
  nucleotide sequence of human IL-7
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, Artificial Sequence
  nucleotide sequence of modified IL-7(M)
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, Artificial Sequence
  nucleotide sequence of modified IL-7(MM)
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, Artificial Sequence
  nucleotide sequence of modified IL-7(MMM)
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, Artificial Sequence
  nucleotide sequence of modified IL-7(MGM)
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, Artificial Sequence
  nucleotide sequence of modified IL-7(DDD)
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, Artificial Sequence
  nucleotide sequence of modified IL-7(MMMM)
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, Artificial Sequence
  nucleotide sequence of modified IL-7(M) fused hyFc
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, Artificial Sequence
  nucleotide sequence of modified IL-7(MM) fused hyFc
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, Artificial Sequence
  nucleotide sequence of modified IL-7(MMM) fused hyFc
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, Artificial Sequence
  nucleotide sequence of modified IL-7(MGM) fused hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, Artificial Sequence
  nucleotide sequence of modified IL-7(MMMM) fused hyFc
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 40 - protein, 4 aa, Artificial Sequence
  Synthetic oligopeptide
MMMM